(19) United States
(12) Patent Application Publication
(10) Pub. No.: US 2025/0381301 A1
(43) Pub. Date: Dec. 18, 2025

(54) Conjugates of Guide RNA-Cas Protein Complex

(71) Applicant: Minghong Zhong, Sellersville, PA (US)

(72) Inventor: Minghong ZHONG, Burlingame, CA (US)

(21) Appl. No.: 17/636,857

(22) PCT Filed: Jun. 10, 2020
(86) PCT No.: PCT/US2020/036860

§ 371 Date: May 20, 2022

Related U.S. Application Data
(60) Provisional application No. 62/888,551, filed on Aug. 19, 2019.
(60) Provisional application No. 62/914,565, filed on Oct. 14, 2019.
(60) Provisional application No. 62/937,876, filed on Nov. 20, 2019.

(51) Int. Cl. A61K 48/00; C12N 9/22; C12N 15/11; C12N 15/86

(52) U.S. Cl. CPC A61K 48/0058; A61K 48/0066; C12N 9/226; C12N 15/11; C12N 15/86; C12N 2310/20; C12N 2740/15043; C12N 2750/14143; C12N 2830/002

(57) ABSTRACT

Provided herein are compositions of conjugates of a guide RNA(s)-CRISPR Cas protein (RNP) complex. The conjugate comprises a guide RNA(s)-CRISPR Cas protein (RNP) complex and one or more molecules selected from PEG, non-PEG polymers, ligands for cellular receptors, lipids, oligonucleotides, polysaccharides and peptides and chemically linked to the Cas protein and/or guide RNA(s). The conjugates are delivered to targeted cells as RNP complexes, or formed in targeted cells from guide RNA conjugates and a mRNA or a viral vector encoding a Cas protein, or formed in targeted cells from a crRNA conjugates and a viral vector encoding both a Cas protein and a tracrRNA. Also provided are preparation methods and uses of these conjugates.

### CROSS REFERENCE TP RELATED APPLICATIONS

The present application claims the benefits of U.S. Provisional Applications Ser. No. 62/888,551, filed on Aug. 19, 2019, Ser. No. 62/914,565, filed on Oct. 14, 2019, and Ser. No. 62/937,876, filed on Nov. 20, 2019, the entire said inventions being incorporated herein by reference.

### INCORPORATION BY REFERENCE OF SEQUENCE LISTING

The contents of the sequence listing text named “RNP_Conjugates_v16_ST25.txt”, which was created on Dec. 15, 2024, and 42,135 bytes in size, are incorporated herein by reference in its entirety.

### TECHNICAL FIELD OF THE INVENTION

The present invention relates to compositions of conjugates of a guide RNA(s)-Cas protein (RNP) complex and their uses as medicinal agents for treatment of viral infectious diseases and as gene regulation, disruption and/or correction-based therapeutics. The conjugate(s) comprises a guide RNA(s)-Cas protein (RNP) complex and one or more molecules selected from a group comprising PEG, non-PEG polymers, ligands for cellular receptors, lipids, oligonucleotides, antibodies, polysaccharides and peptides, and chemically linked to Cas protein and/or guide RNA(s). The guide RNA(s) is chemically modified to increase their stability, enhance the specificity for target recognitions and minimize/eliminate toxicities. The conjugates are delivered to targeted cells as RNP complexes, or formed in targeted cells from guide RNA conjugates and a mRNA or a plasmid or a viral vector encoding a Cas protein, or formed in targeted cells from a crRNA conjugate(s) and a plasmid or a viral vector encoding both a Cas protein and a tracrRNA. These conjugates are useful in improving preciseness in gene editing by driving templated DNA repair, in decreasing or preventing host preexisting immunity to guide RNA-Cas protein complexes by masking epitopes and chemical modifications of guide RNA(s), and also in improving the non-viral delivery of RNP complexes.

### BACKGROUND OF THE INVENTION

The following description of the background is provided simply as an aid in understanding the present disclosure and is not admitted to describe or constitute prior art to the present disclosure.

The CRISPR-Cas system is an adaptive immune system of bacteria and composed of clustered regularly interspaced short palindromic DNA repeats and CRISPR-associated genes that protect bacteria against invading phages and mobile genetic elements. CRISPR-Cas9 is being developed for numerous applications in biotechnology and biomedical research and as a gene therapy agent for treatment of multiple conditions including cancers, infectious diseases, and genetic diseases such as sickle cell anemia and Duchenne's muscular dystrophy (DMD), with 33 trials around the world that involve CRISPR in human cells listed in the NIH's database of global clinical trials to date. Using CRISPR-Cas9 multiplexing gene editing, allogenic universal CAR T cells that are deficient in the TCR beta chain, B2M, PD-1, TCR and CTLA-4 have been produced, with enhanced potency. CRISPR-Cas9 has been applied to silencing/correcting pathogenic proteins in neurodegenerative diseases such as Alzheimer's disease, Huntington's disease, and Parkinson's disease, which should essentially block further progression of symptoms, and it may also be applicable for treatment of dementia with Lewy bodies, frontotemporal dementias, various other tauopathies and amyotrophic lateral sclerosis (ALS). Catalytically impaired Cas9 (dCas9) can target many genomic loci, which has led to technological developments such as base editing, prime editing, epigenetic editing, gene regulation, and chromatin imaging and modeling.

Chronic and/or latent viral infections such as HIV, HBV, and HSV cause enormous suffering, life loss, and financial burdening among the infected individuals. These infectious diseases are incurable, and contagious to variable degrees, and are prominent threats for public health, highlighting the urgent needs for curative therapies. To date, effective antiviral therapies only suppress viral replication but do not clear virus in patients, and do not target the viral genetic materials of latently integrated (e.g. proviral DNA) or non-replicating episomal viral genomes (such as cccDNA) in human cells. Nevertheless, these viral DNAs have been reported to directly cause these chronic or latent infections.

Several reports showed CRISPR-Cas9 as potential antiviral treatments targeting viral genomes such as HBV, HIV, HSV, and Epstein-Barr virus (EBV). Yang and et al. reported the CRISPR/Cas9 system could significantly reduce the production of HBV core and surface proteins in Huh-7 cells transfected with an HBV-expression vector, and disrupt the HBV encoding templates both in vitro and in vivo, indicating its potential in eradicating persistent HBV infection. They observed that two combinatorial gRNAs targeting different sites could increase the efficiency in causing indels. The study by Seeger and Sohn also reported CRISPR/Cas9 efficiently inactivated HBV genes in NTCP encoding HepG2 cells permissive for HBV infection. Wang and Quake observed patient-derived cells from a Burkitt's lymphoma with latent Epstein-Barr virus infection presented dramatic proliferation arrest and a concomitant decrease in viral load after exposure to a CRISPR/Cas9 vector targeted to the viral genome and a mixture of seven guide RNAs at the same molar ratio via plasmid. Hu and et al. reported CRISPR/Cas9 system could eliminate the integrated HIV-1 genome by targeting the HIV-1 LTR U3 region in single and multiplex configurations. It inactivated viral gene expression and replication in latently infected microglial, promonocytic, and T cells, completely excised a 9,709-bp fragment of integrated proviral DNA that spanned from its 5′ to 3′ LTRs, and caused neither genotoxicity nor off-target editing to the host cells. CRISPR-Cas9 has been most recently shown to clear HIV-1 in a subset of humanized mice, when used in combination with long-acting slow-effective release antiretroviral therapy, promising a cure for this so-far incurable disease (Dash, et al. Nat. Comm. 2019, 10, 2753). More studies can be found in a recent review on antiviral applications of CRISPR-Cas9 (Lee, C. Molecules 2019, 24, 1349).

As supported by previous studies using in vitro or in vivo transcribed crRNAs or sgRNAs, targeting genes or viral DNA at multiple sites could enhance the effectiveness, which can be better practiced by delivering a mixture/chemical library of different chemically modified crRNAs, sgRNAs or lgRNAs (including various spacers), targeting multiple sites and/or variants/mutations of a single site in viral genomes equivalent to combination therapies such as HAART.

### SUMMARY OF THE INVENTION

This invention pertains to compositions of conjugates of a guide RNA(s)-Cas protein (RNP) complex and their uses as medicinal agents in treatment of viral infectious diseases and as gene regulation, disruption and/or correction-based therapeutics. The conjugate(s) comprises a guide RNA(s)-Cas protein (RNP) complex and one or more molecules selected from the group comprising PEG, non-PEG polymers, ligands for cellular receptors, lipids, oligonucleotides, antibodies, polysaccharides and peptides, and chemically linked to Cas protein and/or guide RNA(s). The conjugates are delivered to targeted cells as RNP complexes, or formed in targeted cells from guide RNA conjugates and a mRNA or a plasmid or a viral vector encoding a Cas protein delivered by co-injections or separate injections, or formed in targeted cells from a crRNA conjugates and a plasmid or a viral vector encoding both a Cas protein and a tracrRNA delivered by co-injections or separate injections. These conjugates are useful in improving preciseness in gene editing by driving templated DNA repair, in decreasing or preventing host preexisting immunity to guide RNA(s)-Cas protein complexes by masking epitopes and chemical modifications of guide RNA(s), and also in improving the non-viral delivery of RNP complexes.

The guide RNA(s) of said RNP complex conjugates is a chemically modified crRNA, dual guide RNAs (crRNA and tracrRNA), a sgRNA or a IgRNA oligonucleotide comprising nucleotides modified at sugar moieties such as 2′-deoxyribonucleotides, 2′-methoxyribonucleotides, 2′-F-ribonucleotides, 2′-F-arabinonucleotides, 2′-0,4′-C-methylene nucleotides (LNA), unlocked nucleotides (UNA), nucleoside phosphonoacetates (PACE), thiophosphonoacetates (thioPACE), and phosphoromonothioates:

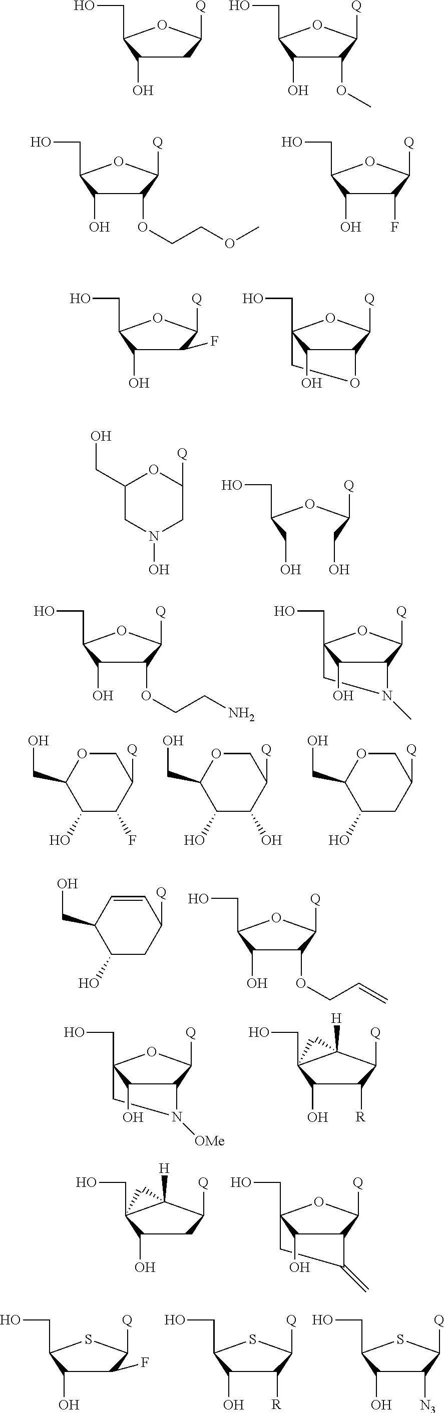

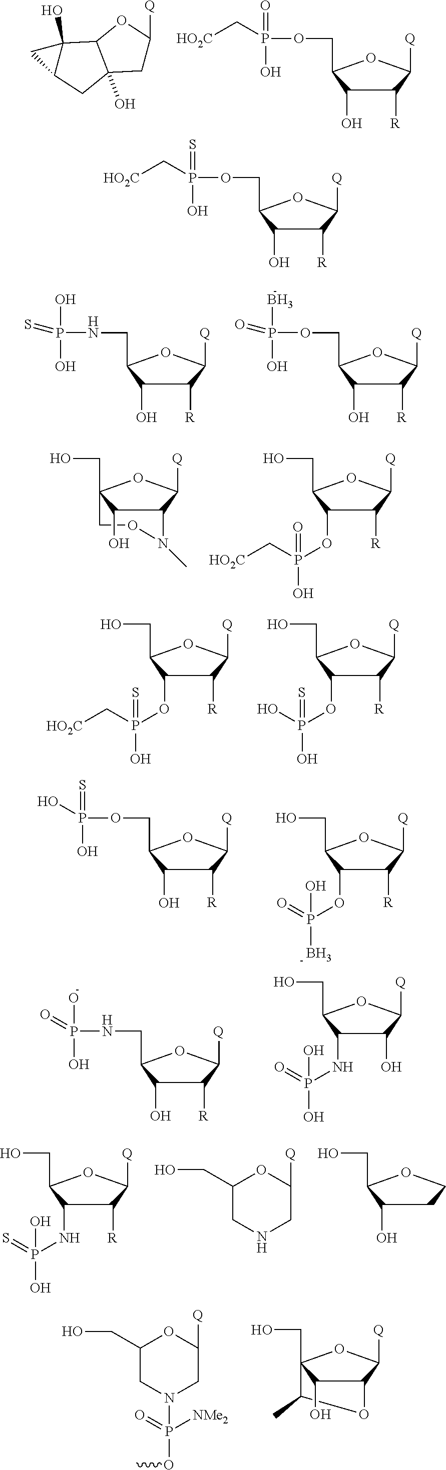

wherein Q is a nucleobase and R is H, OH, F, OMe, or OCH₂CH₂OCH₃; The chemically modified crRNA, sgRNA or lgRNA oligonucleotides optionally comprises modified nucleotide base moieties such as G-clamps, A-clamps and other modified bases:

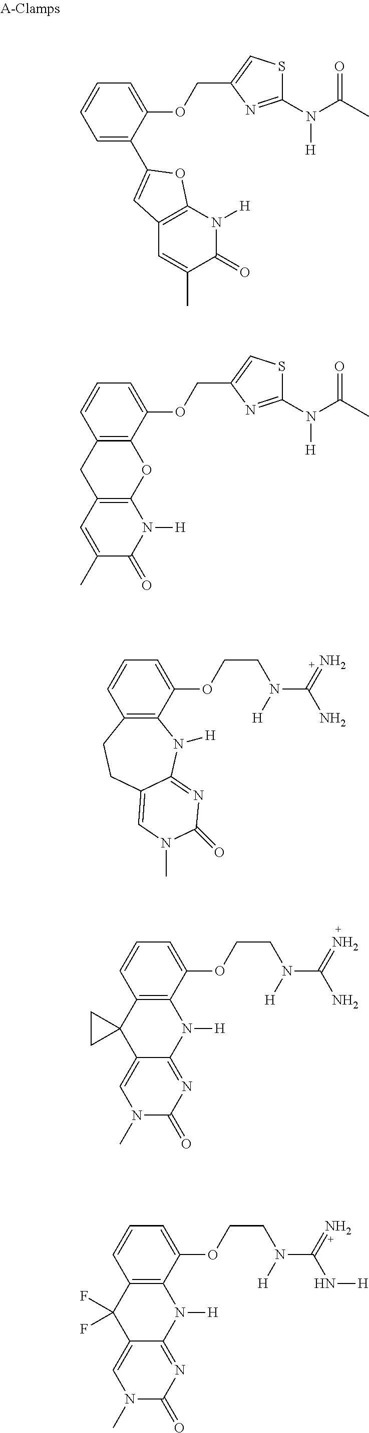 [SMILES: CC(=O)Nc1nc(COc2ccccc2-c2cc3cc(C)c(=O)[nH]c3o2)cs1 ; CC(=O)Nc1nc(COc2cccc3c2Oc2[nH]c(=O)c(C)cc2C3)cs1 ; Cn1cc2c(nc1=O)Nc1c(cccc1OCCNC(=N)N)CC2 ; Cn1cc2c(nc1=O)Nc1c(OCCNC(=N)N)cccc1C21CC1 ; Cn1cc2c(nc1=O)Nc1c(OCCNC(=N)N)cccc1C2(F)F]

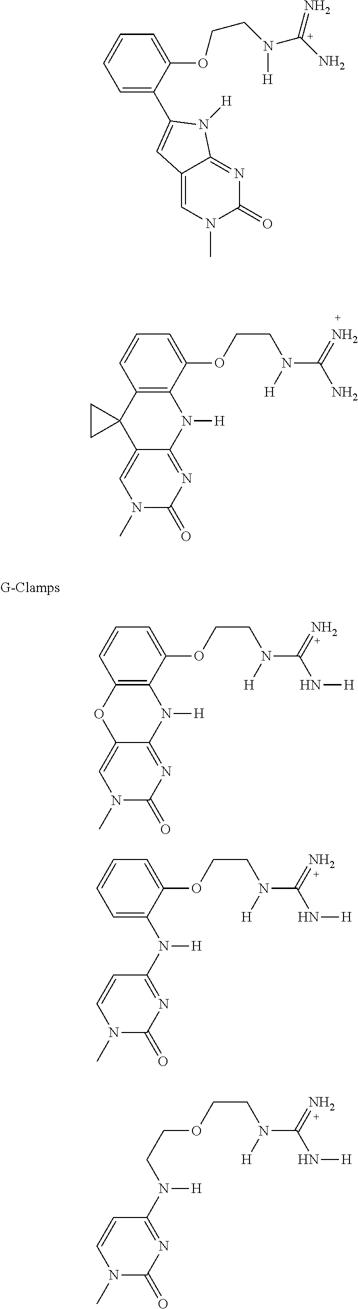

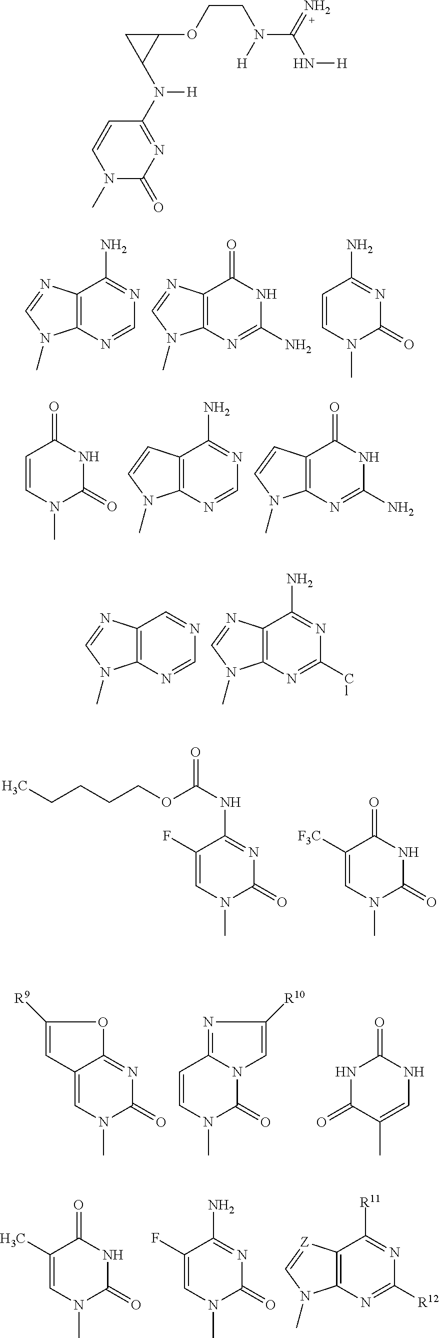

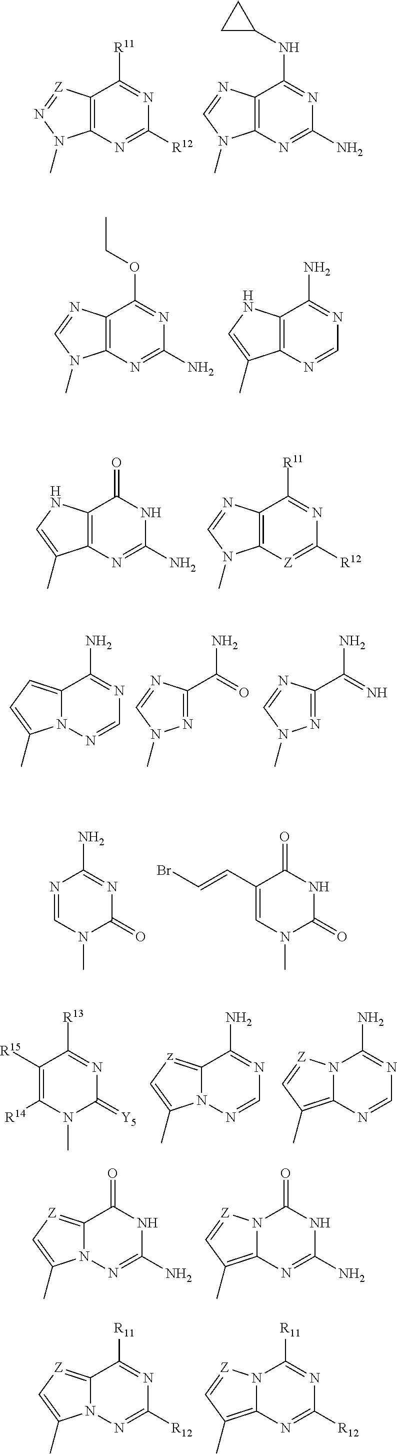

wherein:
(i) Z is N or CR¹⁶; (ii) R⁹, R¹⁰, R¹¹, R¹², R¹³, R¹⁴ and R¹⁵ are independently H, F, Cl, Br, I, OH, OR′, SH, SR′, SeH, SeR′, NH₂, NHR′, NHOH, NHOR′, NROW, NR′₂, NHNH₂, NR′NH₂, NR′NHR′, NHNR′₂, NR′NR′₂, lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, CN, CO₂H, CO₂R′, CONH₂, CONHR′, CONR′₂, CH═CHCO₂H, or CH═CHCO₂R′, wherein R′ is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, chemical modifications of guide RNAs at either sugar or base moiety or both optimize the complementary recognition of the guide-target duplex to improve the cutting efficiency and lower the off-target effects.

In some embodiments, chemical modifications of guide RNAs at either sugar or base moiety or both optimize the shape complementarity between Cas protein and the minor and major grooves of the guide-target duplex to improve the efficiency and lower the off-target effects.

In some embodiments, the chemically modified crRNA, dual guide RNAs, sgRNA or lgRNA oligonucleotide is conjugated with peptides, aptamers, oligonucleotides, antibodies, small molecule receptor ligands such as GalNAc, biotin, cholesterol, tocopherol, lipid, or folate and etc., for selective tissue targeting. The conjugating sites are selected from 3′-end, 5′-end and ligation sites of these oligonucleotides.

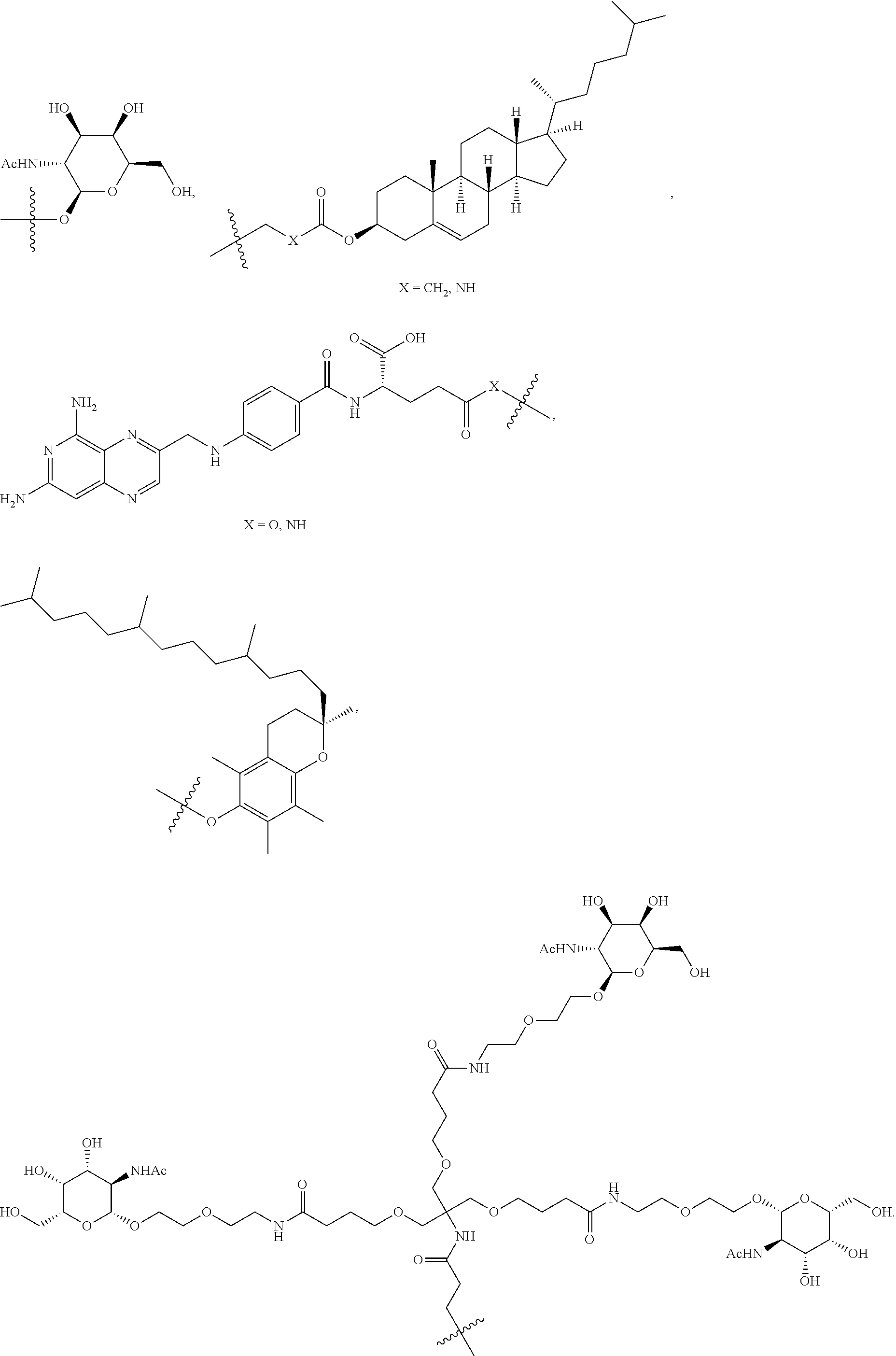

In certain embodiments, the said viral vectors encoding both a Cas protein and a tracrRNA comprise the following elements, optionally in 5′>3′ orientation: a mammalian promoter and optional enhancer, a cDNA encoding a single Cas protein, one or more nuclear localization sequence, a polyadenylation signal, a U6 promotor and the tracrRNA sequence. As an example, an adeno-associated virus (AAV) vector is depicted in FIG. 7 (A).

In certain embodiments, the said viral vectors enabling targeted cells to stably express Cas9 comprise the following elements, optionally in 5′>3′ orientation: a mammalian promoter and optional enhancer, a cDNA encoding a single Cas protein, one or more nuclear localization sequence, and a polyadenylation signal. As an example, an adeno-associated virus (AAV) vector is depicted in FIG. 7 (B).

In some embodiments, the cDNAs encoding Cas9 and/or tracrRNA of the said viral vectors are optimized to encode Cas9 variants and tracrRNAs for both better efficacy and lower off-target and other side effects.

In some embodiments, the said viral vectors and crRNA(s), or lgRNAs or sgRNAs or their conjugates either in an aqueous solution with or without transfection reagents or packaged in a non-viral carrier, are administrated by co-injections or separate injections.

In some embodiments, the conjugates of functional ternary crRNA-tracrRNA-Cas9 complexes are formed cellularly or in vivo by either tracrRNA-Cas9 binary complex formation followed by hybridization of crRNA or crRNA conjugates to the bound tracrRNA via repeat: anti-repeat recognition and interactions with Cas9, or binding of Cas9 to crRNA-tracrRNA complex or its conjugates dimerized via repeat: anti-repeat recognition. The conjugates of functional binary lgRNA/sgRNA-Cas9 complexes are formed in vivo by binding of stably or inducibly expressed Cas9 to exogenous lgRNA or sgRNA or its conjugates.

In certain embodiments, arrayed libraries of structurally optimized chemically modified lgRNAs or crRNAs or their conjugates and cells or animals stably or inducibly expressing a Cas protein or a Cas9-tracrRNA binary complex are used for drug discovery, medical research and biological studies, and genome wide screening.

In some embodiments, the Cas protein is a single protein effector of other class 2 CRISPR systems, such as a Cas12a protein, which is delivered in a tissue tropic viral vector, and chemically modified crRNA(s) or its conjugates are delivered either in an aqueous solution gymnotically or with transfection reagents or packaged in a non-viral carrier by co-injections or separate injections. The single protein effector such as Cas9 and Cas12a can be catalytically inactive and coupled/fused with protein effectors such as transcription activators, transcription repressors, catalytic domains of DNA methyltransferase, histone acetyltransferase and deacetylase, and nucleic acid deaminases, for gene editing and regulations.

In some embodiments, the said vectors and oligonucleotides or their conjugates are administrated to the target cells, i.e. T cells from patients, and the modified cells are infused back to the patients.

In another embodiment, the target tissue or cells are treated with the said vectors encoding the tracrRNA-Cas9 binary complex, and the modified cells are infused back to the patients. The said crRNAs or their conjugates are administrated to activate the CRISPR-Cas9 for regulation, disruption or correction of targeted genes or viral genomes.

In yet another embodiment, the target tissue or cells are treated with the said vectors encoding the Cas9 protein, and the modified cells are infused back to the patients. The said lgRNAs or sgRNAs or their conjugates are administrated to activate the CRISPR-Cas9 for regulation, disruption or correction of targeted genes or viral genomes.

In some embodiments, one viral vector encoding a Cas9 orthologue has the tracrRNA encoded in cis, and crRNA(s) or crRNA conjugates are administrated either in an aqueous solution with or without transfection reagents, or packaged in a non-viral carrier are administrated by co-injections or separate injections with administration ratios of the vector to copies of each crRNA ranging from 1:1 to 1:5. In some embodiments, a single crRNA or its conjugate is administrated. In some embodiments, a mixture of multiple crRNAs or their conjugates is administrated.

In some embodiments, one viral vector encoding a Cas9 orthologue, and lgRNA or lgRNAs or their conjugates either in an aqueous solution with or without transfection reagents, or packaged in a non-viral carrier are administrated by co-injections or separate injections with administration ratios of the vector to copies of each lgRNA ranging from 1:1 to 1:5.

In certain embodiments, the viral vector is selected from engineered adeno-associated virus (AAV), retrovirus, lentivirus, adenovirus vehicles, and etc.

In certain embodiments, the codons for Cas9 protein bearing a C-terminal SV40 nuclear localization signal are codon-optimized for human cells.

In certain embodiments, the expression of Cas9 protein is under the control of a single or a plurality of switchable transcription promotor/enhancer/depressor.

In certain embodiments, the Cas9 protein is selectively delivered to specific tissues based on tissue tropism of the viral vector and cell selective promotor of Cas9 gene.

In certain embodiments, crRNA, dual guide RNAs, sgRNA or lgRNA comprises a spacer selected from sequences of 12˜20 nt of DNA genome of a pathogen, e.g. a virus, bacterium, or other microorganism that causes disease(s), of which each thymine is replaced with uracil, immediately next to a PAM (such as NGG). The spacer RNA oligomer has the same sequence as the sense non-target strand (5′→3′) of the genome immediately next to the protospacer adjacent motif (PAM), and namely is its RNA transcript with or without further chemical modifications. The recognition of the complementary anti-sense target DNA strand by crRNA, sgRNA or lgRNA directs Cas9 DNA endonuclease for site-specific cleavage to form a specific double strand break (DSB) leading to degradation of viral genomes or deadly mutations for the pathogen resulting from DNA repair pathways via non-homologous end joining (NHEJ) and microhomology mediated end joining (MMEJ).

In certain embodiments, crRNA, sgRNA or lgRNA comprises a spacer selected from sequences of 12˜20 nt of HIV genomes, of which each thymine is replaced with uracil, immediately next to a PAM (such as NGG). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of HBV genomes, of which each thymine is replaced with uracil, immediately next to a PAM (such as NGG). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of HSV genomes, of which each thymine is replaced with uracil, immediately next to a PAM (such as NGG). The spacer RNA oligomer has the same sequence as the sense strand (5′→3′) of the genome, and namely is its RNA transcript with or without further chemical modifications.

In certain embodiments, crRNA, sgRNA or lgRNA comprises a spacer selected from sequences of 12˜20 nt of EBV genomes, of which each thymine is replaced with uracil, immediately next to a PAM (such as NGG). The spacer RNA oligomer has the same sequence as the sense strand (5′→3′) of the genome, and namely is its RNA transcript with or without further chemical modifications.

In certain embodiments, crRNAs, sgRNAs and lgRNAs comprise different spacers corresponding to different loci of viral genomes and/or variants of a single locus of target genomes.

In certain embodiments, crRNAs, sgRNAs and lgRNAs comprise spacers selected from sequences of 12˜20 nt of host genes encoding host factors involved in viral entry, transcription/reverse transcription and/or replications, of which each thymine is replaced with uracil, immediately next to a PAM (such as NGG). The spacer RNA oligomers have the same sequences as the sense non-target strands (5′→3′) of the genomes immediately next to the protospacer adjacent motif (PAM), and namely are their RNA transcripts with or without further chemical modifications. The recognition of the complementary anti-sense target DNA strand by crRNAs, sgRNAs or lgRNAs directs Cas9 DNA endonuclease for site-specific cleavage to form a specific double strand break (DSB), introducing mutations in these factors leading to host's resistance to the virus.

In certain embodiments, crRNAs, sgRNAs and lgRNAs comprise spacers selected from sequences of 12˜20 nt of host mutated loci, defective gene or a target gene encoding a protein of defective or partial activity or function, of which each thymine is replaced with uracil, immediately next to a PAM (such as NGG). The spacer RNA oligomers have the same sequences as the sense non-target strands (5′→3′) of the genomes immediately next to the protospacer adjacent motif (PAM), and namely are their RNA transcripts with or without further chemical modifications. The recognition of the complementary anti-sense target DNA strand by crRNAs, sgRNAs or lgRNAs directs Cas9 DNA endonuclease for site-specific cleavage to form a specific double strand break (DSB), allowing a transgene cassette flanked with homologous regions to recombine with the host loci and replace the mutated DNA with the correct sequence.

In certain embodiments, the said transgene cassette is an ssDNA HDR template conjugated to guide RNAs to form a guide RNA(s)-ssDNA conjugate for replacing a mutated DNA with its correct sequence.

In certain embodiments, the said transgene cassette is an ssDNA HDR template conjugated to guide RNAs for editing viral episomal DNAs and integrated viral DNAs in host genes, and the editions are deletions, insertions or point mutations to suppress or eliminate viral protein expression, or overexpression of host pathogenic proteins upregulated by viral DNA integration.

In certain embodiments, the said editions incorporate stop codon(s) and/or cis regulatory elements to suppress or eliminate viral protein expression, or overexpression of host pathogenic proteins upregulated by viral DNA integration.

In certain embodiments, the said guide RNA(s)-ssDNA conjugates are optionally further conjugated with peptides, aptamers, antibodies, small molecule receptor ligands such as GalNAc, cholesterol, tocopherol, lipids, or folate and etc., for selective tissue targeting. The conjugating sites are selected from 3′-end, 5′-end and ligation sites of these said guide RNA(s)-ssDNA conjugates.

The Cas protein is selected from Cas9 variants comprising SpCas9, St1Cas9, SaCas9, NmCas9, and etc. (Jin and et al. Adv. Sci. 2020, 1902312; Doudna, J. A. Nature 2020, 578, 229), and can be a nickase or catalytically inactive Cas9 (dCas9) coupled/fused with protein effectors such as transcription activators, transcription repressors, catalytic domains of DNA methyltransferase, histone acetyltransferase and deacetylase, reverse transcriptase and nucleic acid deaminases, for gene editing and regulations.

The Cas protein can be alternatively any single protein effector of other class 2 CRISPR systems (Type V and VI), such as a Cas12(a, b, c, e, g, h, i and etc.), Cas13 and Cas14 protein. The single protein effector such as Cas12 and Cas14 can be catalytically inactive and coupled/fused with protein effectors such as transcription activators, transcription repressors, catalytic domains of DNA methyltransferase, histone acetyltransferase and deacetylase, and nucleic acid deaminases, for gene editing and regulations.

In certain embodiments, the Cas protein is optionally engineered to introduce conjugating cysteines to replace selected solvent exposed amino acids near to or contained by epitopes by site directed mutagenesis, and cysteines of wild type enzymes (e.g. C80 and C573 of Cas9) are optionally mutated to avoid potential deactivation of enzymes due to conjugations at these cysteines.

In certain embodiments, the guide RNA(s)-Cas protein (RNP) complex(es) is conjugated with molecules selected from PEG, non-PEG polymers, ligands for cellular receptors, antibodies, lipids, oligonucleotides, polysaccharides, glycans and peptides.

In certain embodiments, the Cas conjugating sites are selected from surface/solvent exposed/protruded amino acid residues such as lysine, arginine, serine, cysteine, aspartate, or glutamate of Cas proteins or an amino acid(s), e.g. a cysteine, introduced by site-directed mutagenesis for selective conjugations to mask or shield epitopes.

In certain embodiments, the guide RNA(s)-Cas protein (RNP) complex(es) is conjugated at either Cas protein, or guide RNAs, or both.

In certain embodiments, the guide RNA(s)-Cas protein (RNP) complex(es) is PEGylated.

In certain embodiments, the guide RNA(s)-Cas protein (RNP) complex(es) is PEGylated with more than two PEG polymers to shield/mask epitopes.

In certain embodiments, the guide RNA(s)-Cas protein (RNP) complex(es) are conjugated with other non-PEG polymers, ligands for cellular receptors, lipids, oligonucleotides, antibodies, polysaccharides, glycans or peptides.

In certain embodiments, PEG-conjugated guide RNA-Cas protein (RNP) complex(es) is further covalently linked to other non-PEG polymers, ligands for cellular receptors, lipids, oligonucleotides, antibodies, polysaccharides, glycans or peptides.

In certain embodiments, guide RNA(s)-Cas protein (RNP) complex(es) is covalently linked to one or more molecules selected from PEG, non-PEG polymers, ligands for cellular receptors, lipids, oligonucleotides, antibodies, polysaccharides, glycans and peptides.

In certain embodiments, guide RNA(s)-conjugates, e.g. IgRNA-ssDNA conjugates, are delivered with an mRNA or a vector encoding a Cas protein.

In certain embodiments, guide RNA(s)-conjugates, e.g. IgRNA-ssDNA conjugates, are delivered to cells stably or inducibly expressing a Cas protein.

In certain embodiments, lgRNA-ssDNA conjugates are used to drive the templated repair of CRISPR edited DNA upon hybridization of ssDNA with the PAM distal fragment of the R-loop asymmetrically released from Cas9:lgRNA:DNA complexes and thus to promote fast release of the RNP complex from the edited DNA to increase its turnover frequency (TOF).

In certain embodiments, lgRNA-ssDNA conjugates are used to drive the templated repair of CRISPR edited DNA upon hybridization of ssDNA with the 3′-OH fragment of the R-loop asymmetrically released from Cas:lgRNA:DNA complexes and thus to promote fast release of the RNP complex from the edited DNA to increase its turnover frequency (TOF).

### DETAILED DESCRIPTION OF THE INVENTION

The most commonly used type of CRISPR system for gene regulation, disruption or correction to date is type II represented predominantly by Cas9. CRISPR-Cas9 is a naturally occurring defense system of bacteria. The CRISPR Cas9 endonuclease is activated by the binding of crRNA:tracrRNA and responds specifically to the DNA sequence (target strand) complementary to the spacer in crRNA and cleaves it upon the recognition of a protospacer adjacent motif (PAM) on the 3′-end of the non-target DNA strand. The presence of tracrRNA and Cas9 is required for processing pre-crRNA into individual crRNA by a double-stranded RNA specific ribonuclease, RNase III, forming crRNA:tracrRNA duplexes. This duplex was fused into an artificial single guide RNA (sgRNA via nucleotide tetraloop or lgRNA via a non-Nucleotide linker) for genome engineering purpose and other applications.

Attractive alternatives include other class 2 CRISPR systems such as Type V and VI.

An aspect of the invention is directed to compositions of PEGylated guide RNA(s)-Cas protein complex(es) and uses as medicinal agents in treatment of viral infectious diseases and as gene regulation, disruption and/or correction based therapeutics. The PEG polymers can be other polymers, ligands for cellular receptors, lipids, oligonucleotides, antibodies, polysaccharides or peptides, conjugated to Cas protein and/or guide RNA(s).

In some embodiments, the crRNAs, sgRNAs and lgRNAs are chemically modified for optimization for better efficiency in cleaving target DNAs such as viral genomic DNAs and for minimizing off-target cleavages of host genomic DNAs with or without engineering Cas proteins.

In some embodiments, the crRNAs, sgRNAs or lgRNAs are chemically modified and conjugated with one or more ssDNA donor template.

In some embodiments, the lgRNA-Cas protein complex conjugates are administrated to edit pathogenic genes, of which the lgRNA comprises one or more chemically ligated ssDNA donor template including optional chemical modifications.

In some embodiments, the pathogenic genes are episomal and integrated viral DNA.

In some embodiments, the pathogenic genes are mutated host genes or any genes to be edited.

An aspect of the invention is directed to compositions of viral vectors encoding a tracrRNA-Cas protein binary complex, and chemically modified oligonucleotides of crRNAs or their conjugates, both of which are delivered to the same targeted cells, preparation methods of said compositions, and uses as therapeutic agents in treatment of viral infectious diseases and as gene regulation, disruption and/or correction based therapeutics, and a method to deliver a tracrRNA-Cas protein binary complex in a tissue tropic viral vector and chemically modified crRNA(s) or its conjugate with cell targeting ligands either in an aqueous solution gymnotically or with transfection reagents or in a non-viral carrier by co-injections or separate injections.

Another aspect of the invention is directed to compositions of viral vectors encoding a Cas protein, and chemically modified oligonucleotides of lgRNAs, sgRNAs or their conjugates delivered to the same targeted cells, and a method to deliver a Cas protein in a tissue tropic viral vector and chemically modified lgRNA(s), sgRNAs or their conjugates with cell targeting ligands either in an aqueous solution gymnotically or with transfection reagents or in a non-viral carrier by co-injections or separate injections, in treatment of viral infectious diseases and as gene disruption and/or correction based therapeutics.

Yet another aspect of the invention is directed to compositions of viral vectors encoding a Cas protein, which functions in the absence of a tracrRNA, and chemically modified oligonucleotides of crRNAs or their conjugates, delivered to the same targeted cells, a method to deliver Cas protein in a tissue tropic viral vector and chemically modified crRNA(s) or their conjugates either in an aqueous solution gymnotically or with transfection reagents or in a non-viral carrier by co-injections or separate injections, in treatment of viral infectious diseases and as gene disruption and/or correction based therapeutics.

In some embodiments, the crRNA, sgRNA and lgRNA or their conjugates are chemically modified for optimization for better efficiency in cleaving target DNAs such as viral genomic DNAs and for minimizing off-target cleavages of host genomic DNAs with or without engineering Cas proteins.

In some embodiments, the Cas protein is engineered, or a CRISPR-associated protein of smaller size thus more amenable to viral delivery in human cells, for efficient administrations and better dosage forms.

In other embodiments, the said compositions are administrated, either alone or in combination with small molecule therapeutic agents, therapeutic proteins such as antibodies, or nuclei acids such as mRNAs, antisense oligonucleotides and small interfering RNAs.

### BRIEF DESCRIPTION OF THE DRAWINGS

FIG. 1: shows schematic structure of a Cas9/lgRNA complex conjugate (A) in upper diagram; schematic illustration of guide RNAs conjugated with tris-GalNAc ligand(s) as an example (B) at the bottom.

FIG. 2: shows schematic structure of a PEGylated Cas9/lgRNA complex with guide RNAs conjugated with tris-GalNAc as an example.

FIG. 3: shows examples of methods to prepare a PEGylated Cas9/lgRNA complex.

A. Selective conjugations at serine and lysine residues of a preformed RNP complex; B. Selective conjugations at lysine residues of a preformed RNP complex; C. Selective conjugations at cysteine residues of an engineered Cas protein obtained by site-directed mutagenesis, followed by RNP formation.

FIG. 4: shows a model of a Cas9/lgRNA-ssDNA in synchronizing actions with host enzymes. Host DNA polymerase extends the PAM distal strand of the R-loop asymmetrically released from a Cas9:lgRNA:DNA complex after hybridization with a conjugated ssDNA and uses the ssDNA as the template.

FIG. 5: shows a multiple-turnover STAR (Seek-Tag-Amend-Release) CRISPR-Cas for editing pathogenic genes with enhanced release of the acting RNP complex. 1. Seek: a Cas9/lgRNA-ssDNA complex binds a double-stranded DNA sequence that contains a sequence match to the first 17-20 nucleotides of the lgRNA and immediately before a protospacer adjacent motif (PAM) to form a R-loop. 2. Tag: HNH cleaves the target strand at the position 3 bases upstream of the PAM, while less precise cleavage and further 3′-end processing by RuvC leave shortened PAM distal strands of various lengths which are asymmetrically released from a Cas9:lgRNA-ssDNA:DNA complex. The released DNA strand acts as a primer and hybridizes with the 3′-homology arm of the conjugated ssDNA, which acts as a template for DNA repair. 3. Amend: the tagged double strand breaks are repaired and the flaps are removed and gaps are ligated by cellular enzymes. 4. Release: Cas9:lgRNA-ssDNA complex is released from the repaired DNA, and is ready for next cycle. The Cas9 protein can be a nickase, e.g. HNH (H840A), and a DNA nick is formed and repaired in a similar way (STAR) to release the dCas9:lgRNA-ssDNA.

FIG. 6: shows shape complementarity of Cas9-gRNA:DNA duplex recognitions (A); Binding at major and minor grooves by hydrogen bonds (B); Example of a bound G-Clamp (C); Complementary hydrogen bonding in major and minor grooves of A-Clamps and G-Clamps (D).

FIG. 7: shows AAV vectors encoding a Cas9:tracrRNA complex (A) and a Cas9 protein (B), respectively. The promotors are tissue selective human promotors, which can be inducible, and Cas9 can be a dCas9-effector fusion protein or Cas9 nickase, or any other Cas protein.

FIG. 8: shows division of ternary Cas9:crRNA:tracrRNA RNP complexes into variable crRNA(s) and a fixed binary Cas9:tracrRNA RNP complex for its cellular delivery, and Cas9 can be a dCas9-effector fusion protein or Cas9 nickase, or any other Cas protein. A binary Cas9:tracrRNA RNP complex can be prepared in vitro and delivered to target cells (b. and c.) or formed from a Cas9 protein and tacrRNA in target cells (a.). The binary Cas9:tracrRNA RNP complex forms a ternary Cas9:crRNA:tracrRNA RNP complex upon addition of crRNA(s) or its conjugates (d.). Ternary Cas9:crRNA:tracrRNA RNP complexes and their conjugates can also be alternatively prepared in vitro, and are delivered to target cells (e. and f.).

FIG. 9: shows schematic depiction of administration of AAV vectors and crRNA/crRNA conjugates (A), lgRNA/lgRNA conjugates (B) or a mixture of mRNA(Cas9-NLS) and lgRNA-conjugates packaged in lipid nanoparticles (C) by injections.

FIG. 10: shows schematic depiction of administration of AAV vectors and crRNA/crRNA conjugates (A), lgRNA/lgRNA conjugates (B) or a mixture of mRNA(Cas9-NLS) and lgRNA-conjugates packaged in lipid nanoparticles (C) by IV injections and the formation of RNP complex conjugates in the liver.

FIG. 11: shows schematic depiction of ex vivo adoptive cell therapy (ACT): allogeneic universal CAR-T cells as an example.

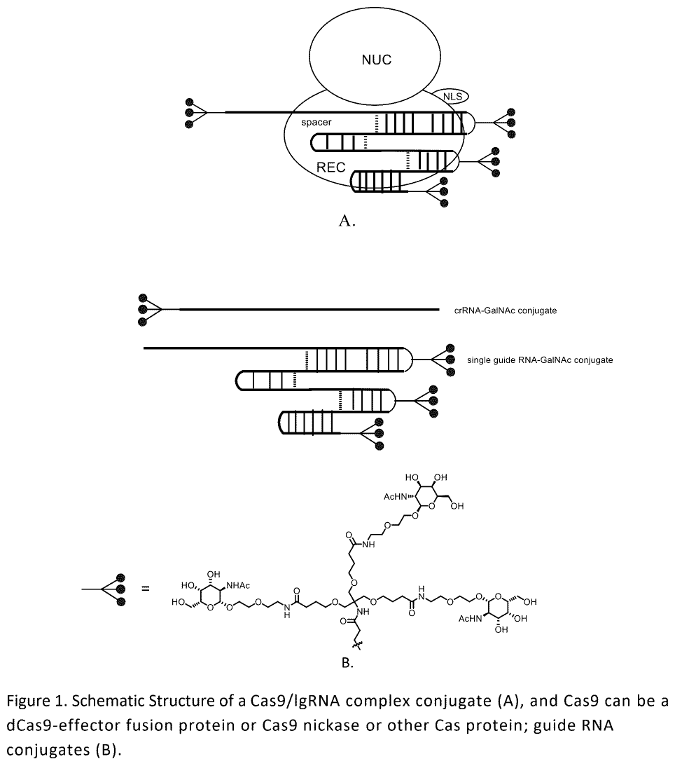

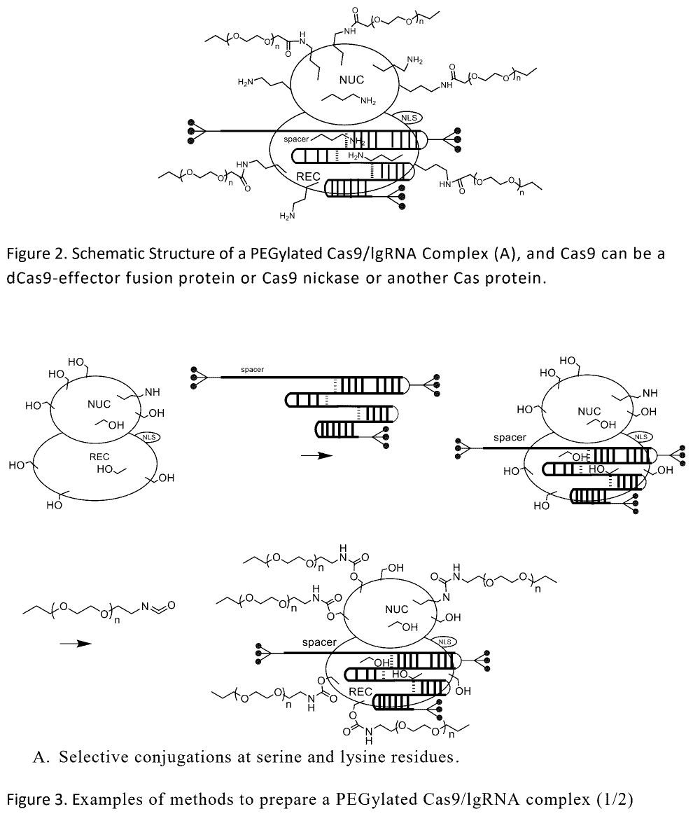

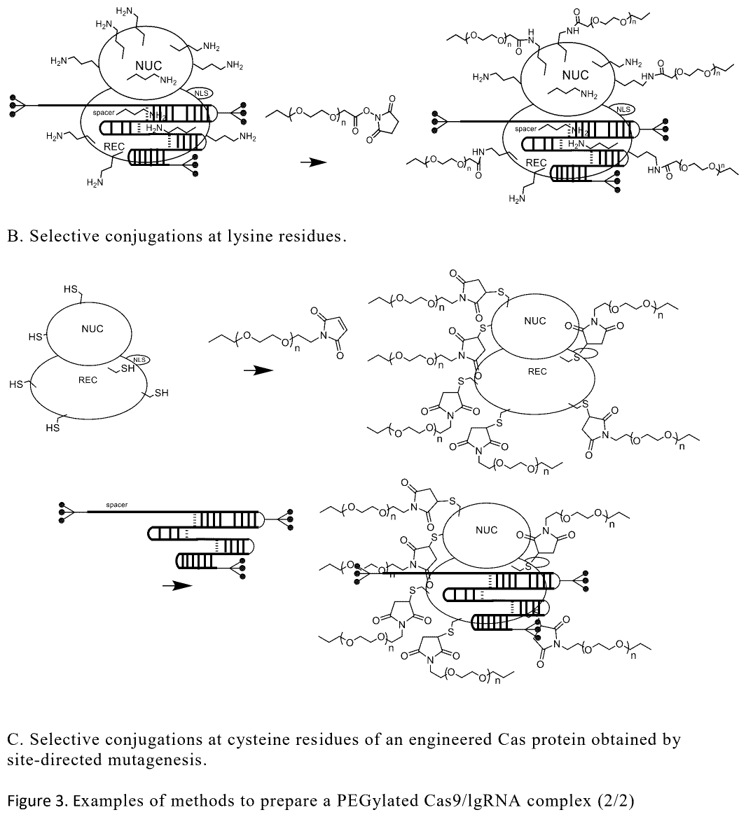

### Definition

The definitions of terms used herein are consistent to those known to those of ordinary skill in the art, and in case of any differences the definitions are used as specified herein instead.

The term “nucleoside” as used herein refers to a molecule composed of a heterocyclic nitrogenous base, containing an N-glycosidic linkage with a sugar, particularly a pentose. An extended term of “nucleoside” as used herein also refers to acyclic nucleosides and carbocyclic nucleosides.

The term “nucleotide” as used herein refers to a molecule composed of a nucleoside monophosphate, di-, or triphosphate containing a phosphate ester at 5′-, 3′-position or both. The phosphate can also be a phosphonate, phosphoramidate, phosphorodiamidate, phosphonoacetate (PACE), thiophosphonoacetate (thioPACE), or phosphoromonothioate.

The term of “oligonucleotide” (ON) is herein used interchangeably with “polynucleotide”, “nucleotide sequence”, and “nucleic acid”, and refers to a polymeric form of nucleotides of any length, either deoxyribonucleotides or ribonucleotides, or analogs thereof. An oligonucleotide may comprise one or more modified nucleotides, which may be imparted before or after assembly of such an oligonucleotide. The sequence of nucleotides may be interrupted by non-nucleotide components.

The term of “CRISPR-Cas system” refers a prokaryotic immune system that confers resistance to foreign genetic elements such as those present within plasmids and phages that provides a form of acquired immunity. RNA harboring the spacer sequence helps Cas (CRISPR-associated) proteins recognize and cut foreign pathogenic DNA. Other RNA-guided Cas proteins cut foreign RNA. CRISPR are found in approximately 50% of sequenced bacterial genomes and nearly 90% of sequenced archaea. The system is being engineered for gene regulation and editing, insertion, disruption and/or correction in eukaryotic cells.

The term of “CRISPR/Cas9” refers to the type II CRISPR-Cas system such as spCas9 from Streptococcus pyogenes. The type II CRISPR-Cas system comprises protein Cas9 and two noncoding RNAs (crRNA and tracrRNA). These two noncoding RNAs were further fused into one single guide RNA via a tetraloop (sgRNA) and a chemically ligated guide RNA via one or more nNt-Linkers (lgRNA). The Cas9/sgRNA or Cas9/lgRNA complex binds double-stranded DNA sequences that contain a sequence match to the first 17-20 nucleotides of the guide RNA(s) and immediately before a protospacer adjacent motif (PAM). Once bound, two independent nuclease domains (HNH and RuvC) in Cas9 each cleaves one of the DNA strands 3 bases (HNH) or more (RuvC) upstream of the PAM, leaving a DNA double stranded break (DSB).

The term of “Cas protein” refers to a class 2 CRISPR-Cas protein.

The term of “off-target effects” refers to non-targeted cleavage of the genomic DNA target sequence by Cas9 or any other Cas protein in spite of imperfect matches between the gRNA sequence and the genomic DNA target sequence. Single mismatches of the gRNA can be permissive for off-target cleavage by Cas9. Off-target effects were reported for all the following cases: (a) same length but with 1-5 base mismatches; (b) off-target site in target genomic DNA has one or more bases missing (‘deletions’); (c) off-target site in target genomic DNA has one or more extra bases (‘insertions’).

The term of “guide RNA” (gRNA) refers to a synthetic fusion of crRNA and tracrRNA via a tetraloop (GAAA) (defined as sgRNA) or other chemical linkers such as an nNt-Linker (defined as lgRNA), and is used interchangeably with “chimeric RNA”, “chimeric guide RNA”, “single guide RNA” and “synthetic guide RNA”. The gRNA contains secondary structures of the repeat:anti-repeat duplex, stem loops 1-3, and the linker between stem loops 1 and 2.

The term of “dual RNAs” or “dual guide RNAs” refers to hybridized complex of the short CRISPR RNAs (crRNA) and the trans-activating crRNA (tracrRNA). The crRNA hybridizes with the tracrRNA to form a crRNA:tracrRNA duplex, which is loaded onto a Cas protein to direct the cleavage of cognate DNA sequences bearing appropriate protospacer-adjacent motifs (PAM).

The term of “lgRNA” refers to guide RNA (gRNA) joined by chemical ligations to form non-nucleotide linkers (nNt-linkers) between crgRNA and tracrgRNA, or at other sites.

The terms of “dual lgRNA”, “triple lgRNA” and “multiple lgRNA” refer to hybridized complexes of the synthetic guide RNA fused by chemical ligations via non-nucleotide linkers. Dual tracrgRNA is formed by chemical ligation between tracrgRNA1 and tracrgRNA2 (RNA segments of ˜30 nt), and crgRNA (˜30 nt) is fused with a dual tracrgRNA to form a triple lgRNA duplex, which is loaded onto Cas9 to direct the cleavage of cognate DNA sequences bearing appropriate protospacer-adjacent motifs (PAM). Each RNA segment can be readily accessible by chemical manufacturing and compatible to extensive chemical modifications.

The term “guide sequence” refers to the about 20 bp sequence within the guide RNA that specifies the target site and is herein used interchangeably with the terms “guide” or “spacer”.

The term “tracr mate sequence” may also be used interchangeably with the term “direct repeat(s)”.

The term of “crgRNA” refers to crRNA equipped with chemical functions for conjugation/ligation. The oligonucleotide may be chemically modified close to its 3′-end, any one or several nucleotides, or for its full sequence. A crgRNA may also be prepared by in vitro transcription at the presence of a RNA polymerase such as bacteriophage T7 RNA polymerase, and the conjugating chemical function, e.g., amine and alkyne, is incorporated at its 5′ (preferably as 5′-GU or 5′-GC) or 3′-end from a nucleoside triphosphate analogue, e.g. CTP, UTP:

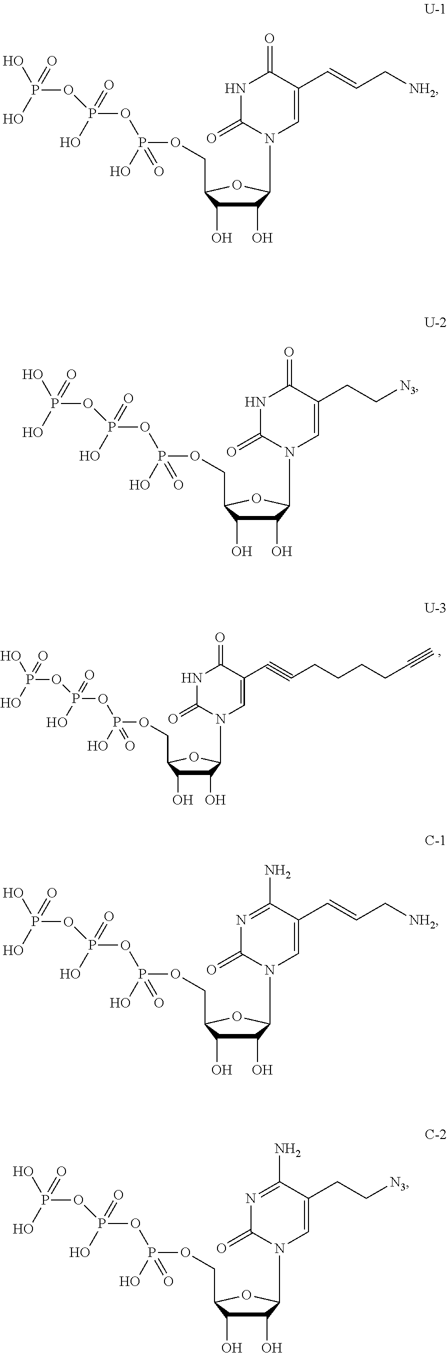

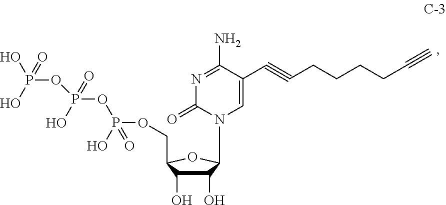

and etc.

The term of “tracrgRNA” refers to tracrRNA equipped with chemical functions for conjugation/ligation. The oligonucleotide may be chemically modified at any one or several nucleotides, or for its full sequence by chemical synthesis. A tracrgRNA may also be prepared by in vitro transcription at the presence of a RNA polymerase such as bacteriophage T7 RNA polymerase, and the conjugating chemical function, e.g., amine and alkyne, is incorporated at its 5′ (preferably as 5′-GU or 5′-GC) or 3′-end from a nucleoside triphosphate analogue.

The term of “the protospacer adjacent motif (PAM)” refers to a DNA sequence immediately following the DNA sequence targeted by Cas9 in the CRISPR bacterial adaptive immune system, including NGG, NNNNGATT, NNAGAA, NAAAC, and others from different bacterial species where N is any nucleotide. In CRISPR-Casl2a system, “PAM” refers to a DNA sequence such as TTTN immediately before the targeted DNA sequence.

The term of “chemical ligation” refers to joining together synthetic oligonucleotides via an nNt-linker by chemical methods such as click ligation (the azide-alkyne reaction to produce a triazole linkage), thiol-maleimide reaction, and formations of other chemical groups.

The term of “complementary” refers to the ability of a nucleic acid to form hydrogen bond(s) with another nucleic acid sequence by either traditional Watson-Crick or other non-traditional types. Cas9 contains two nuclease domains, HNH and RuvC, which cleave the DNA strands that are complementary and non-complementary to the 20 nucleotide (nt) guide sequence in crRNAs, respectively.

The term of “a donor template” refers to a transgene cassette or a gene-editing-sequence flanked with homologous regions to recombine with the host loci and replace the mutated DNA with the correct sequence by HDR/SSTR. A donor template can be an ssDNA or a dsDNA or a plasmid/vector, and may be chemically conjugated to guide RNA(s) or Cas protein via a covalent linker.

A donor template can be chemically synthesized and equipped with chemical functions for conjugations/ligations. A conjugating donor template may also be prepared by in vitro gene synthesis at the presence of a DNA polymerase, with chemical functions, e.g. an amine and an alkyne, enzymatically incorporated at its 5′ or 3′-end for chemical conjugation/ligation from a nucleoside triphosphate analogue.

The term of “gene editing sequence”, “gene-editing-sequence” or “gene_editing_sequence” refers to the sequence contained in a donor template sequence to introduce expected gene editing, between the two homology arms identical to the DNA fragments flanking the cleavage site.

The term of “Hybridization” refers to a reaction in which one or more polynucleotides form a complex that is stabilized via hydrogen bonding between the bases of the nucleotide residues. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy)methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl and thexyldimethylsilyl, ester groups, examples of such being formyl, (C1-C10) alkanoyl optionally mono-, di- or tri-substituted with (C1-C6) alkyl, (C1-C6) alkoxy, halo, aryl, aryloxy or haloaryloxy, the aroyl group including optionally mono-, di- or tri-substituted on the ring carbons with halo, (C1-C6) alkyl, (C1-C6) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and G.M. Wuts, T.W. Greene, “Protective Groups in Organic Synthesis”, John Wiley & Sons Inc., Hoboken, New Jersey, 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl, Boc, Cbz, benzoyl (Bz), N,N-dimethylformamidine (DMF), trityl, and benzyl (Bn). See also G. M. Wuts, T. W. Greene, “Protective Groups in Organic Synthesis”, John Wiley & Sons Inc., Hoboken, New Jersey, 2007, and related publications.

The term “conjugation”, as used herein, refers to a method for covalently crosslinking drug molecules, proteins or nucleic acids to other molecules using crosslinking reagents. The product of conjugation is referred as “conjugate(s)”. Traditional pharmaceuticals can be linked to monoclonal antibodies to deliver targeted doses, prevent breakdown, decrease immunogenicity, and increase bioavailability in circulation. CRISPR RNP complex can be chemically modified by linking to other molecules either covalently or non-covalently.

The term “conjugating site”, as used herein, refers to a chemical moiety which is directly linked to other molecules by conjugation, and a conjugating site can be an amino acid residue, N-terminus or C-terminus of proteins, a nucleoside, a nucleotide, or a phosphate.

The term “PEG,” or “macrogol”, as used herein, refers to polyethylene glycol chains, linear, branched, substituted or unsubstituted. A derivatized linear single PEG chain comprises at least 2 PEG subunits.

The term “PEGylation”, as used herein, refers to the process of both covalent and non-covalent attachment or amalgamation of polyethylene glycol (PEG) polymer chains to molecules and macrostructures, such as a drug, a CRISPR RNP complex, a therapeutic protein or vesicle, which is then described as PEGylated. PEGylation is routinely achieved by incubation of a reactive derivative of PEG with the target molecule.

The term “glycan”, as used herein, refers to polysaccharides or the carbohydrate portion of a glycoconjugate, such as a glycoprotein, glycolipid, or a proteoglycan, even if the carbohydrate is only an oligosaccharide

The term “polysaccharides”, as used herein, refers compounds consisting of a large number of monosaccharides linked glycosidically.

The term “epitope” or “antigenic determinant”, as used herein, refers to the part of an antigen that is recognized by the immune system, specifically by antibodies, B cells, or T cells. An epitope can be either conformational or linear.

The term “epitope masking”, as used herein, refers to identifying potentially immunogenic peptide sequences and modifying or removing them to prevent detection by the immune system while still maintaining the therapeutic function of the original protein.

The term of “Isotopically enriched” refers to a compound containing at least one atom having an isotopic composition other than the natural isotopic composition of that atom. The term of “Isotopic composition” refers to the amount of each isotope present for a given atom, and “natural isotopic composition” refers to the naturally occurring isotopic composition or abundance for a given atom. As used herein, an isotopically enriched compound optionally contains deuterium, carbon-13, nitrogen-15, and/or oxygen-18 at amounts other than their natural isotopic compositions.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent known to be useful for, or which has been or is currently being used for the treatment or prevention of a disorder or one or more symptoms thereof.

The term of “gene therapy” refers to altering a disease-causing gene in a patient or introducing a healthy copy of a mutated gene to a patient to treat genetic diseases. CRISPR/Cas can potentially be used to introduce site specific gene editing to correct disease-causing mutations, or to deliver a correct gene into human genome to fix a defect gene or a desired gene. CRISPR/Cas can potentially be used to remove and/or deactivate episomal HBV cccDNA and integrated viral genomes such as HIV proviral DNA and integrated HBV DNA to cure these infectious diseases.

It is noted that as used, the singular forms “a,” “an,” and “the” include plural referents unless the context clearly dictates otherwise. Thus, for example, reference to “a guide RNA(s)-Cas protein (RNP) complex” includes a plurality of such complexes. Reference to “the conjugate” includes reference to one or more conjugates and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### Nucleotides

In some embodiments, the crRNA and tracrRNA are truncated at 3′-end and 5′-end, respectively:

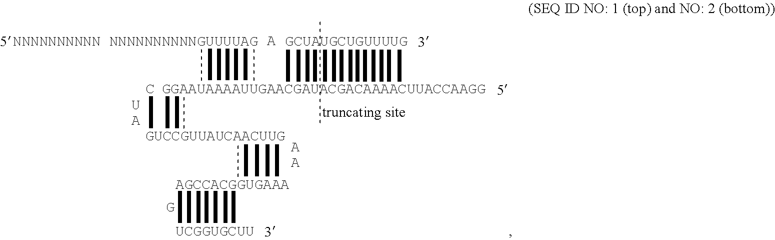

and the duplex ends are rejoined by a small molecule non-nucleotide linker (nNt-linker, ligation1) to form a ligated dual lgRNA:

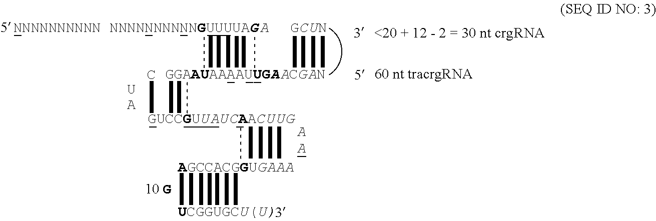

wherein “NNNNNNNNNN NNNNNNNNNN” is a guide sequence of 17-20 nt, and N is preferably a ribonucleotide with intact 2′-OH, and wherein “ [symbol] ” is a chemical nNt-linker.

In some embodiments, the duplex ends are rejoined by a tetraloop (e.g. GAAA) to form an sgRNA:

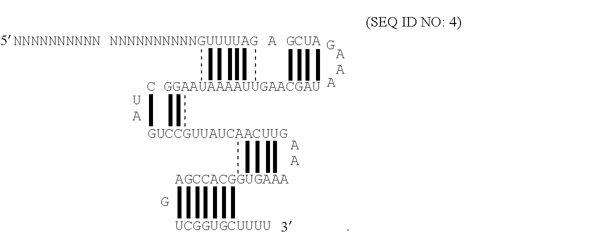

In other embodiments, tracrRNA is a ligated dual oligonucleotide (via ligation2, the inner ligation between tracrgRNA1 and tracrgRNA2), or a multiple oligonucleotide. Non-limiting examples include:

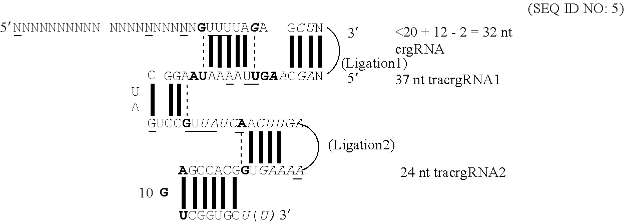

In some embodiments, the crRNA and tracrRNA are further truncated, and the ligation sites can be located at other positions, as illustrated by non-limiting examples of resulting lgRNAs in U.S. Pat. No. 10,059,940.

In some embodiments, crRNA and tracrRNA are truncated at 3′-end and 5′-end, respectively, and the duplex ends are rejoined by nucleotide linker such as an aptamer and thus to provide an extended single gRNA with small molecule and protein recognition module(s):

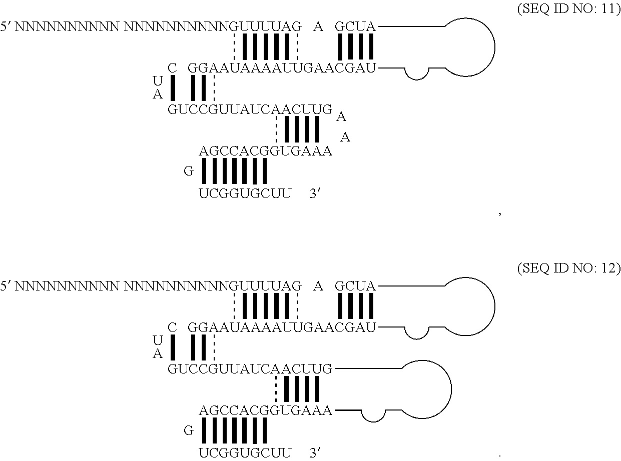

In another embodiment, the crRNA and tracrRNA are truncated at 3′-end and 5′-end, respectively, and the duplex ends are rejoined by non-nucleotide linker-aptamer conjugate to provide an extended single lgRNA with small molecule and protein recognition module(s):

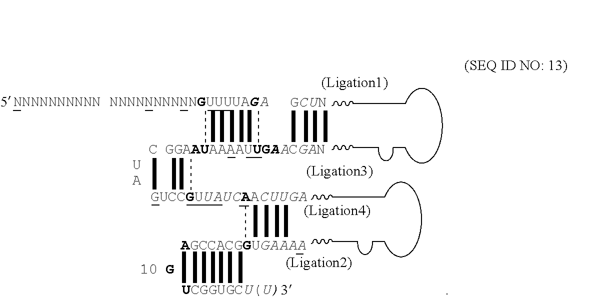

In another embodiment, the stem loop of tracrRNA is split at the GAAA tetraloop, and the duplex ends are rejoined by non-nucleotide linker-aptamer conjugate to provide an extended tracrRNA with small molecule and protein recognition module(s):

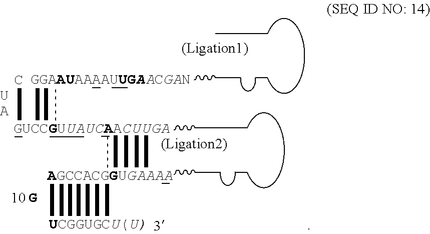

In yet another embodiment, lgRNA is conjugated with an aptamer by a non-nucleotide linker at either of the two GAAA tetraloops or both, or 5′/3′-end of sgRNA, to bind a small molecule or a biopolymer such as a protein or a nucleic acid:

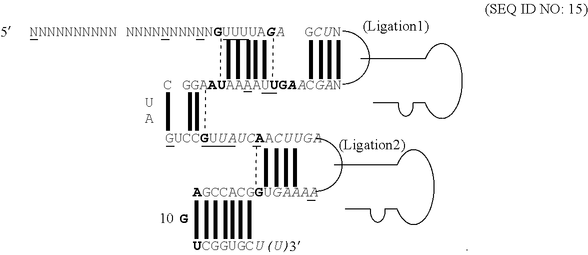

In some embodiments, the crRNA and tracrRNA are shortened by truncation at 3′-end and 5′-end, respectively, and the repeat/anti-repeat duplex comprises a bulge and >12 Watson-Crick base pairs:

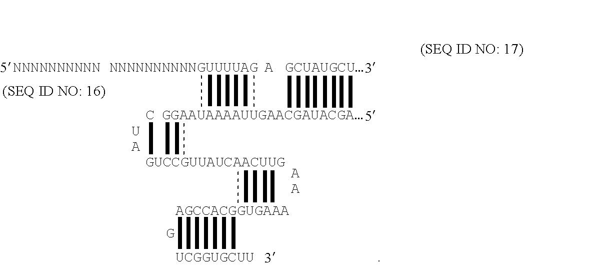

In some embodiments, the crRNA and tracrRNA are joined at 3′-end of tracrRNA and 5′-end of crRNA by a nucleotide linker or a non-nucleotide linker to form a sgRNA or lgRNA, respectively; and the tracrRNA is optionally a ligated tracrRNA comprising one or more than one non-nucleotide linker:

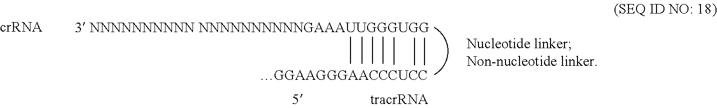

In some embodiments, the dual guide RNAs, sgRNA and lgRNA are conjugates of small molecule ligands, antibodies or aptamers:

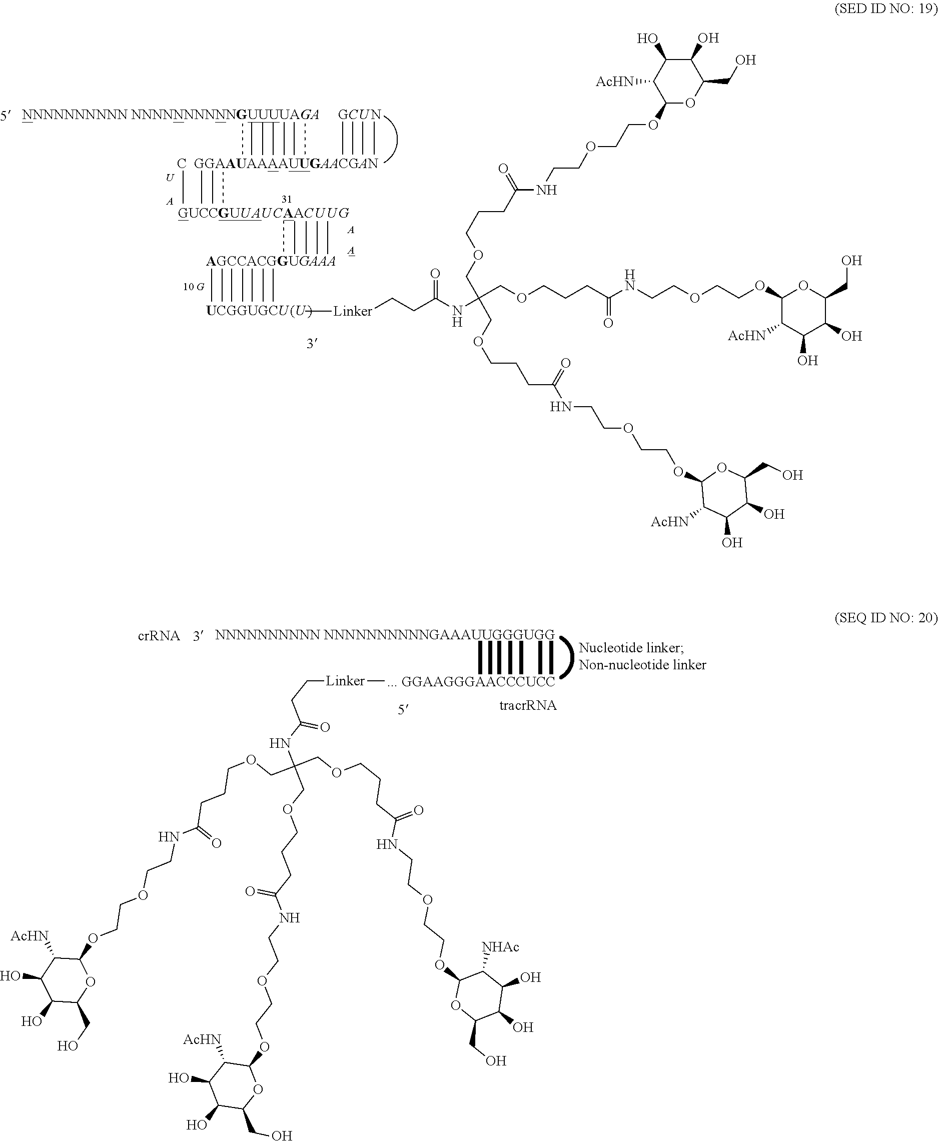

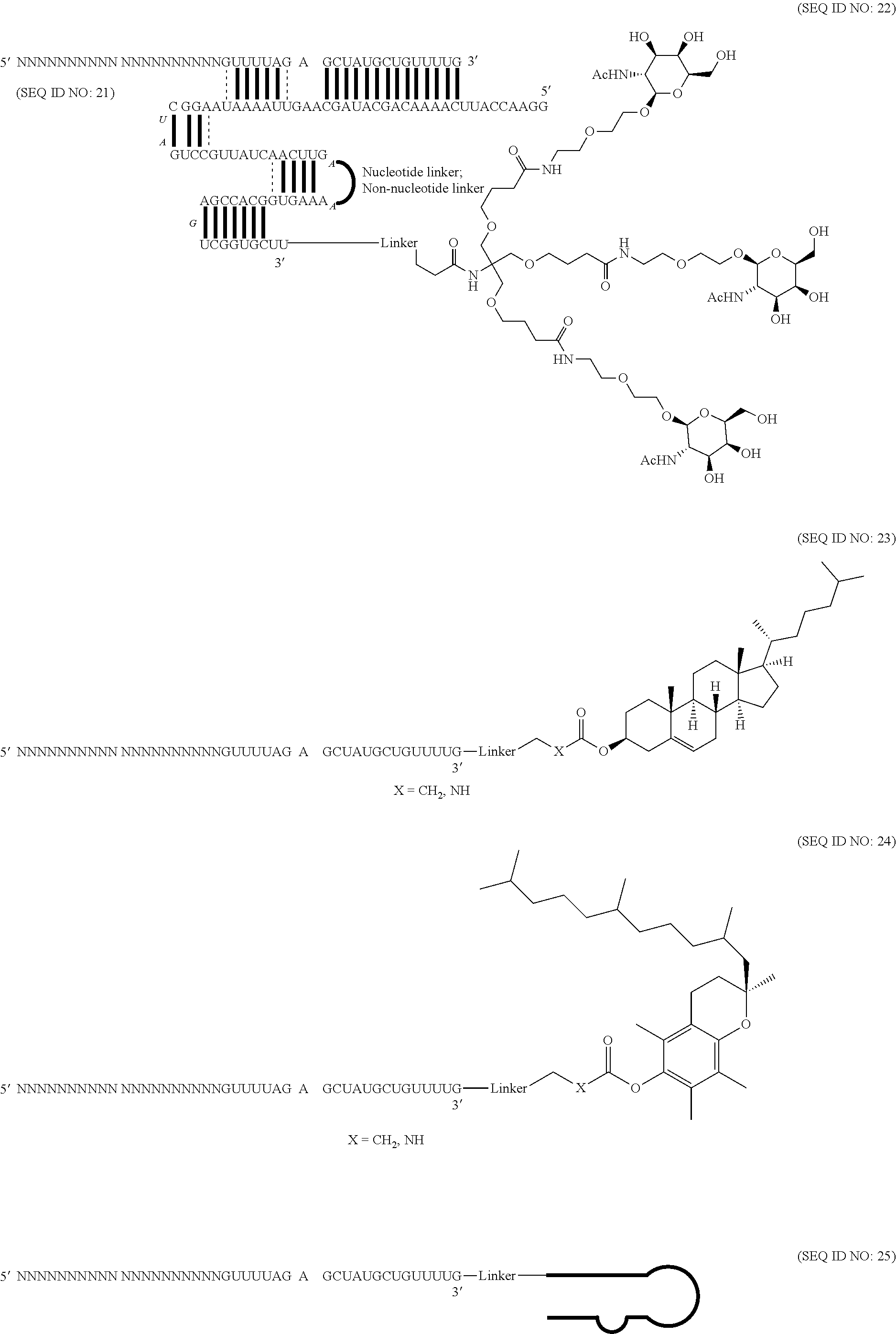

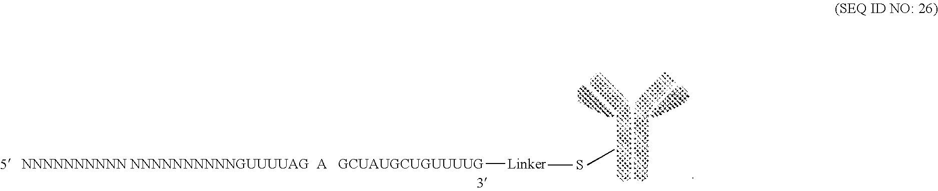

In some embodiments, the crRNA, dual guide RNAs, sgRNA and lgRNA are conjugates of single-stranded template repair (SSTR) donor DNA templates of sequences containing a 5′-homology arm and a downstream gene editing sequence, optionally followed by a 3′-homology arm. Non-limiting such examples are given below:

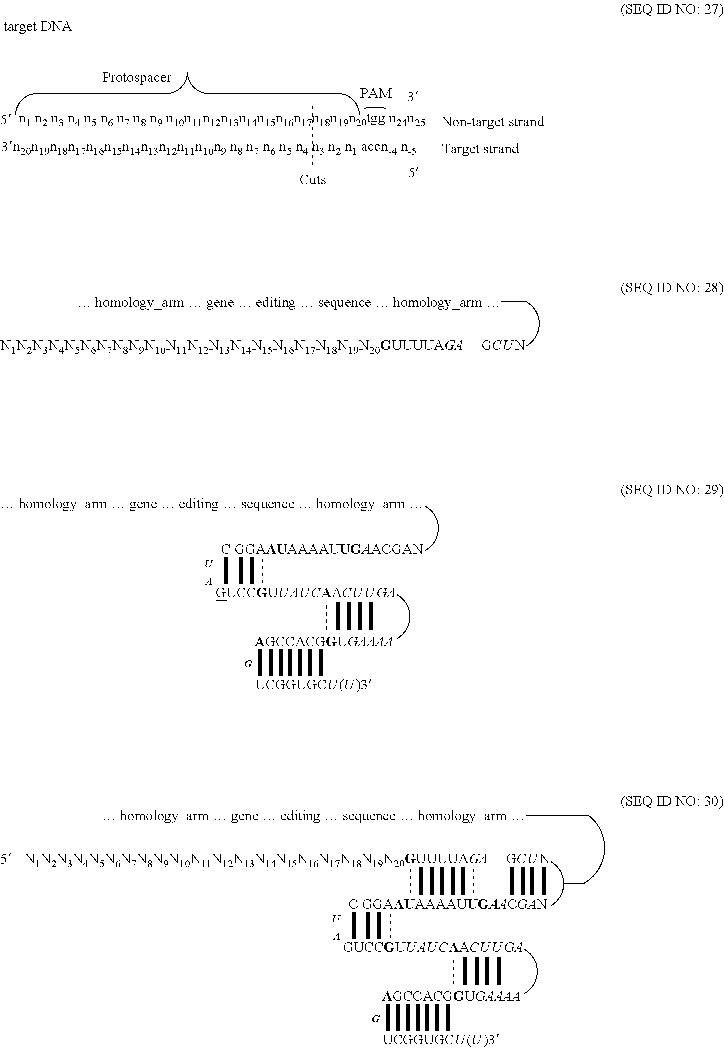

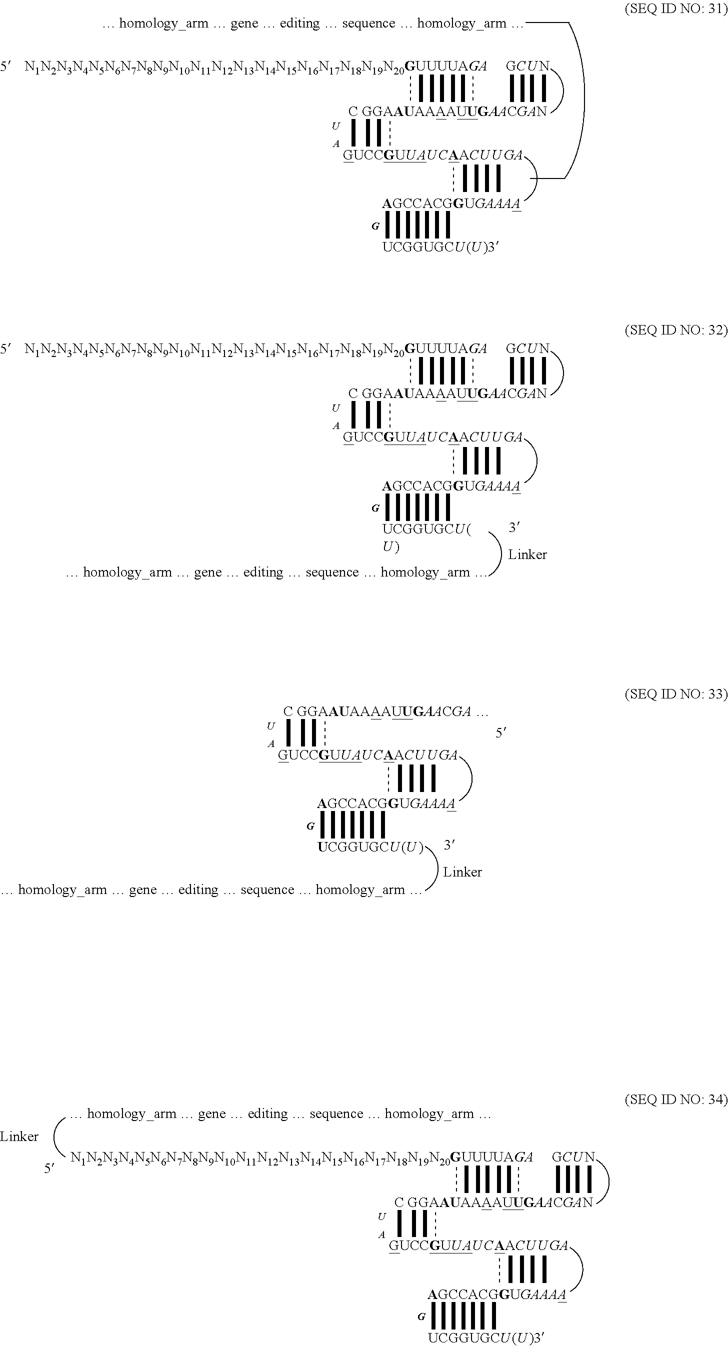

In certain embodiments, the conjugating linker is an oligonucleotide.

In certain embodiments, the conjugating linker is an RNA tetraloop such as ANYA, CUYG, GNRA, UNAC and UNCG, in which N could be uracil, adenine, cytosine, or guanine, R is either guanine or adenine and Y is either uracil or cytosine.

In certain embodiments, the conjugating linker is an nNt-linker, as described in U.S. Pat. No. 10,059,940, selected from a group comprising a 1,4-disubstituted 1,2,3-triazole formed between an alkyne and an azide via [3+2]cycloaddition catalyzed by Cu(I) or without Cu(I) catalysis (such as strain-promoted azide-alkyne cycloaddition (SPAAC)), a thioether by chemical ligation between a thiol and a maleimide, and other functional groups.

In certain embodiments, homology arms flanking the gene editing sequence overlap with the non-target PAM containing strand of target DNA.

In certain embodiments, homology arms flanking the gene editing sequence overlap with the target strand of target DNA.

In some embodiments, the conjugating site is the 5-position of the nucleoside base U or C, or the 7-position of the nucleoside base 7-deazaguanine or 7-deazaadenine, or the 8-position of the nucleoside base purine.

In other embodiments, the conjugating site is the 2′- or 5′-position of the sugar moiety of the 5′-end nucleotide of crRNA, tracrRNA, a single strand HDR/SSTR donor template (ssDNA), sgRNA or lgRNA.

In other embodiments, the conjugating site is the 2′- or 3′-position of the sugar moiety of the 3′-end nucleotide of crRNA, tracrRNA, a single strand HDR/SSTR donor template (ssDNA), sgRNA or lgRNA.

In certain embodiments, as non-limiting examples, the lgRNA-ssDNA conjugates comprise the following structures:

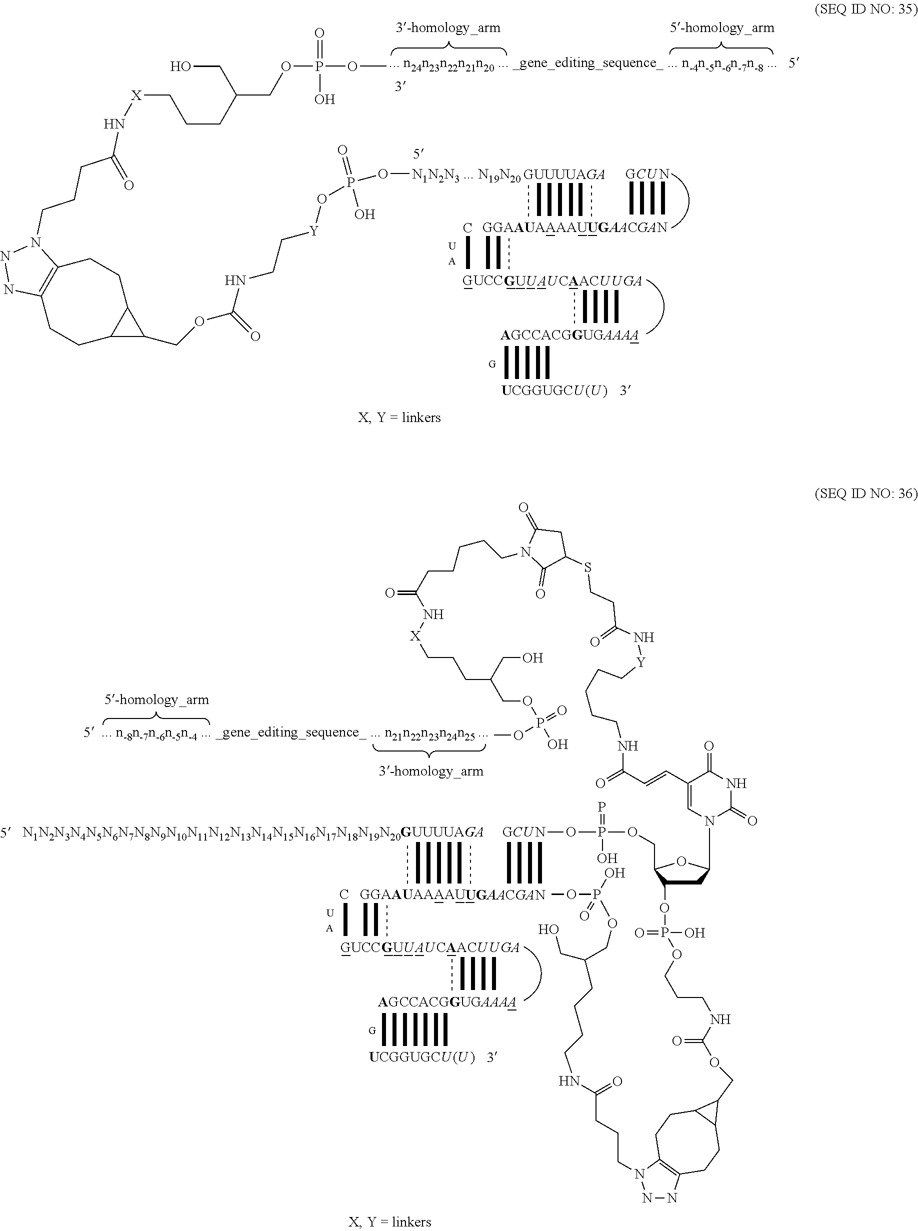

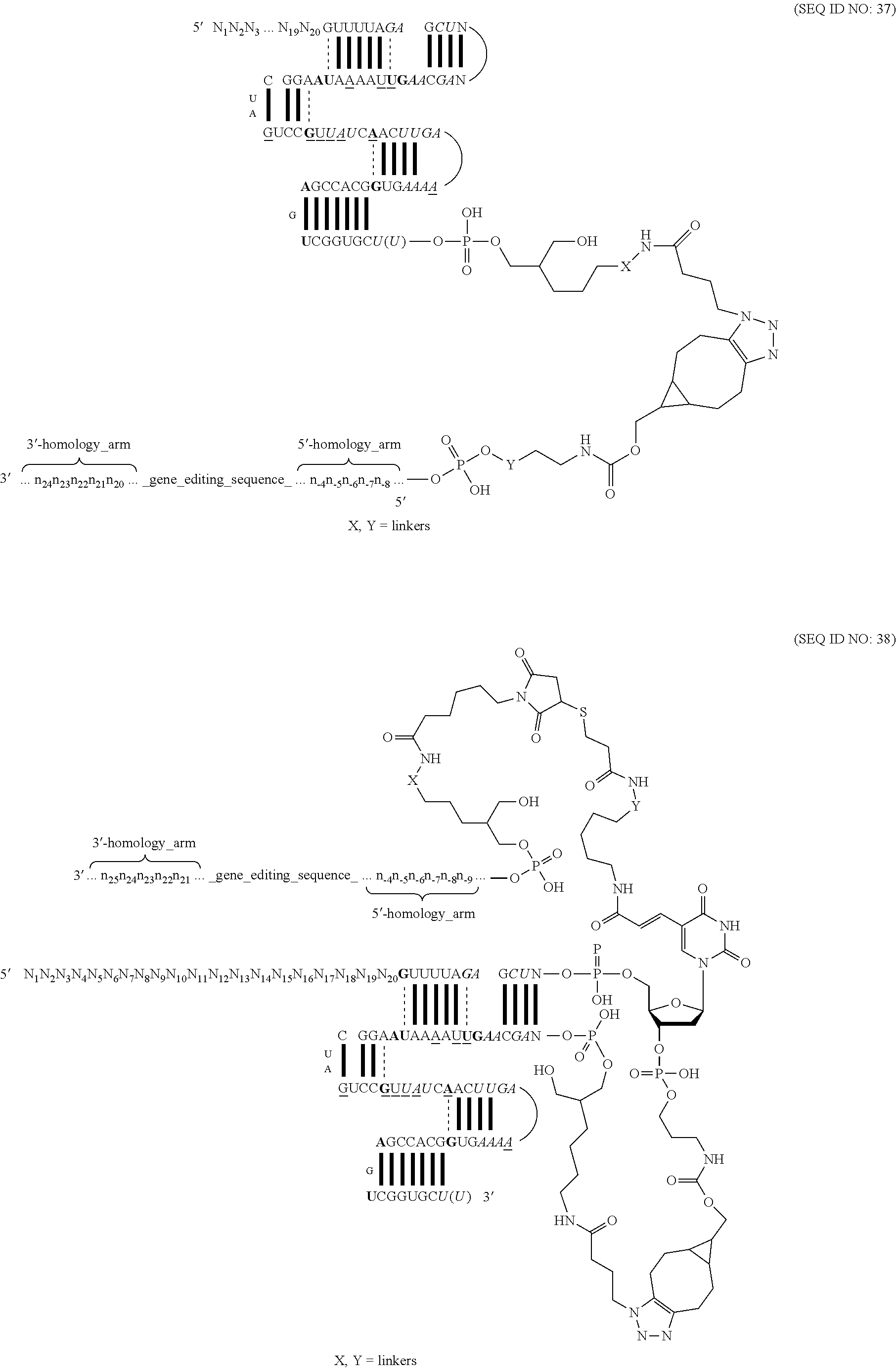

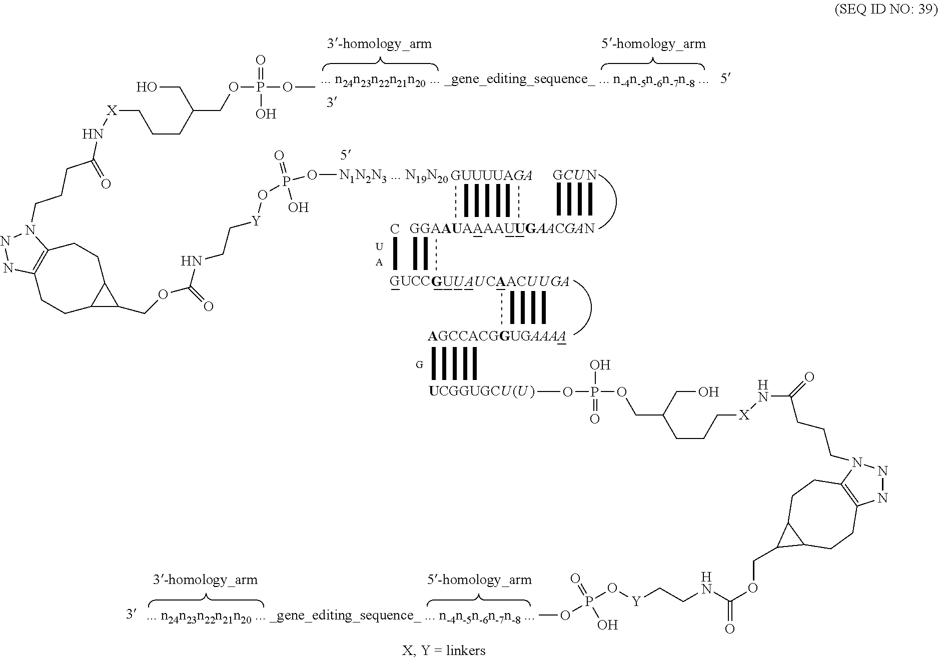

In some embodiments, the “gene-editing-sequence” comprises one or more sequences selected from a group comprising 5′-(tga)-3′, 5′-(taa)-3′, 5′-(tag)-3′, 5′-(tga-ntga-ntga)-3′, 5′-(tga-ntga-ntaa)-3′, 5′-(tga-ntga-ntag)-3′, 5′-(tga-ntaa-ntga)-3′, 5′-(tga-ntaa-ntaa)-3′, 5′-(tga-ntaa-ntag)-3′, 5′-(tga-ntga-ntga)-3′, 5′-(tga-ntga-ntaa)-3′, 5′-(tga-ntga-ntag)-3′, 5′-(taa-ntga-ntga)-3′, 5′-(taa-ntga-ntaa)-3′, 5′-(taa-ntga-ntag)-3′, 5′-(taa-ntaa-ntga)-3′, 5′-(taa-ntaa-ntaa)-3′, 5′-(taa-ntaa-ntag)-3′, 5′-(taa-ntga-ntga)-3′, 5′-(taa-ntga-ntaa)-3′, 5′-(taa-ntga-ntag)-3′, 5′-(tag-ntga-ntga)-3′, 5′-(tag-ntga-ntaa)-3′, 5′-(tag-ntga-ntag)-3′, 5′-(tag-ntaa-ntga)-3′, 5′-(tag-ntaa-ntaa)-3′, 5′-(tag-ntaa-ntag)-3′, 5′-(tag-ntga-ntga)-3′, 5′-(tag-ntga-ntaa)-3′ and 5′-(tag-ntga-ntag)-3′, wherein n is any nucleotide, and said more stop codons comprises repetitive said sequence separated by absent or more nucleotides in between or different said sequences separated by absent or more nucleotides in between.

In some embodiments, the “gene-editing-sequence” comprises one or more sequences such as stop codons to deactivate integrated and episomal viral DNAs, oncogenic or pathogenic gene loci in animals and human.

In some embodiments, the “gene-editing-sequence” comprises one or more sequences such as stop codons to deactivate target gene(s) and to eliminate deleterious off-target edits.

In some embodiments, the “gene-editing-sequence” comprises DNA sequence(s) to correct oncogenic or pathogenic gene mutations in animals and human.

In other embodiments, the conjugated single strand HDR/SSTR donor template (ssDNA) forms a duplex with its complementary stand, i.e. HDR donor template is a double strand DNA (dsDNA) covalently linked to guide RNA(s) via a linker to either of its double strands.

The said linker of X and Y is an L linker, a nucleotide linker or an nNt-linker and can be the same or different.

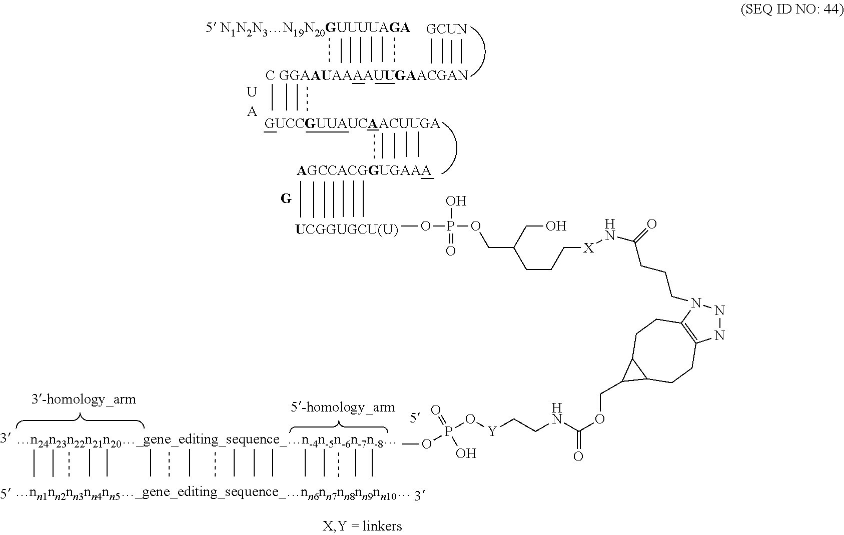

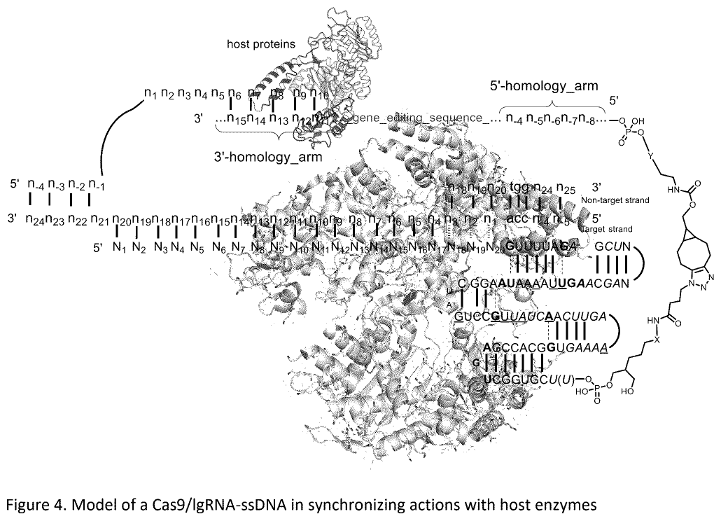

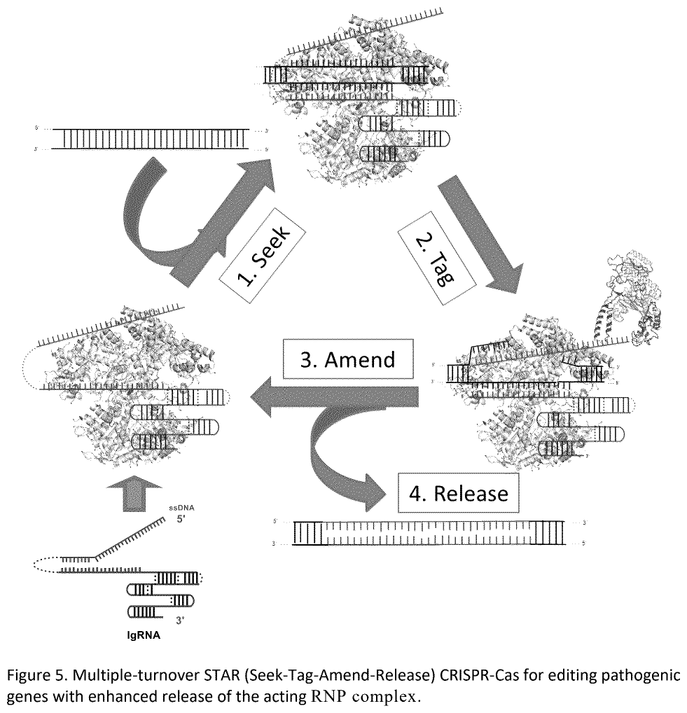

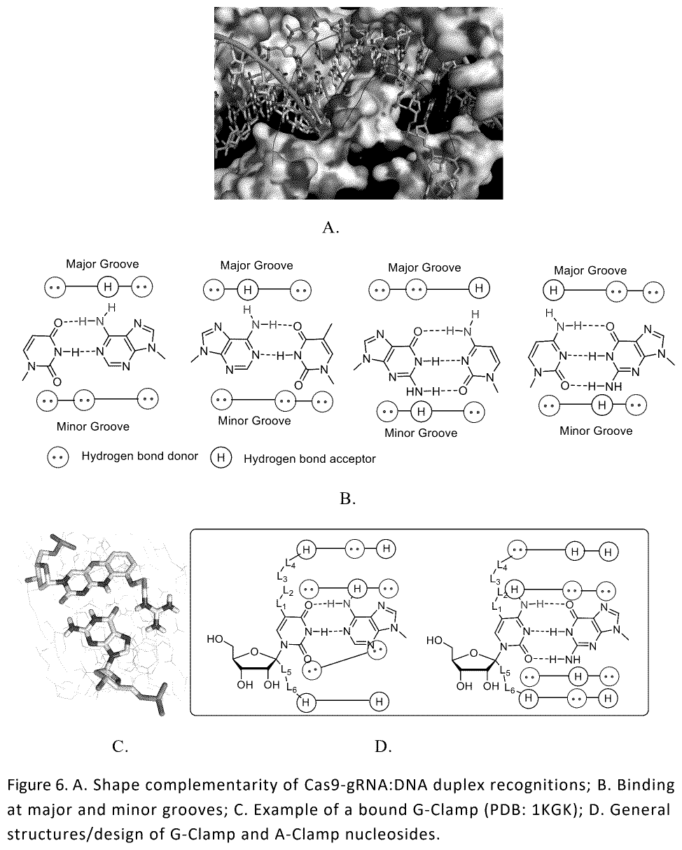

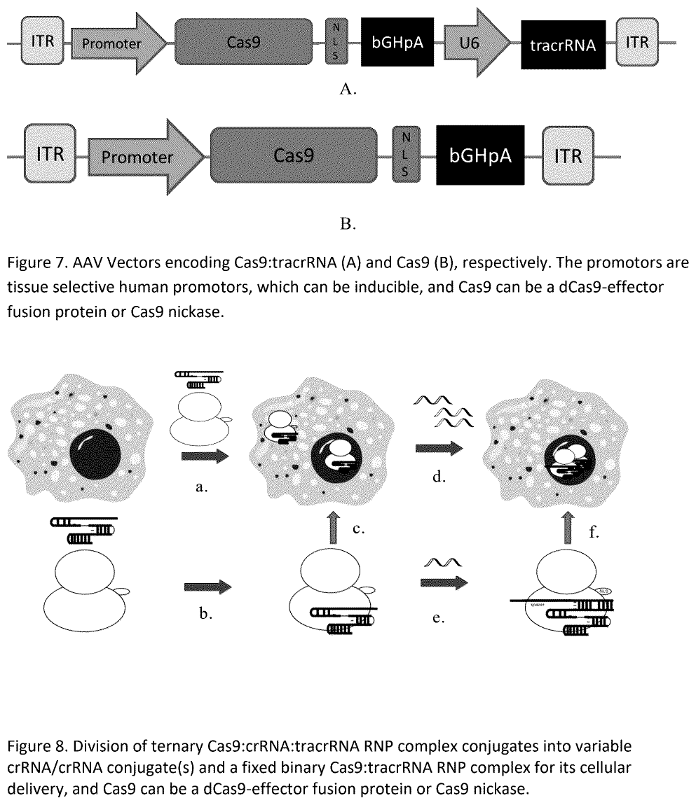

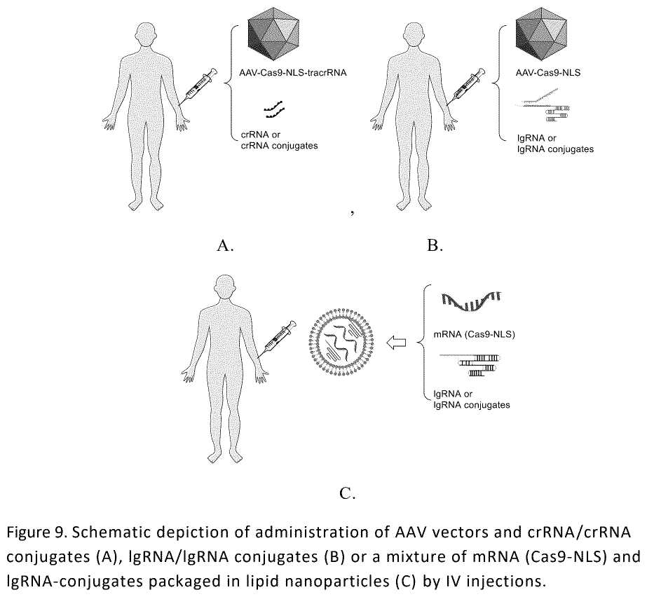

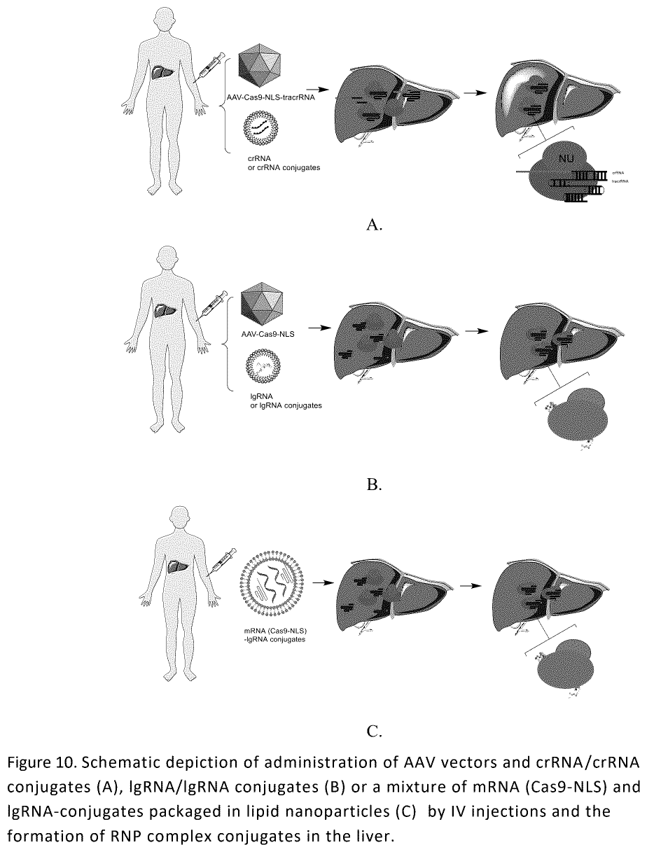

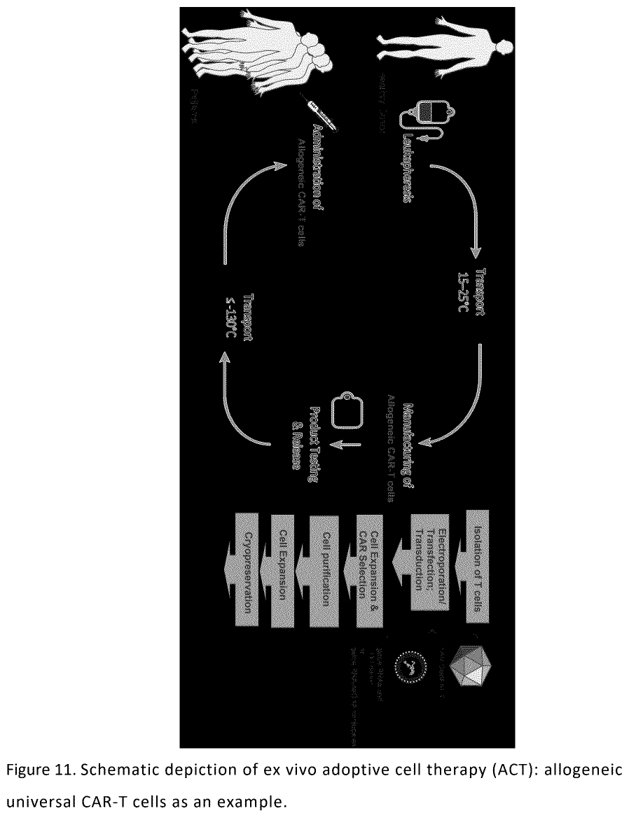

In some embodiments, the said conjugated oligonucleotide is a donor template which comprises two sequences overlapping with the target strand or with the non-target strand of the DNA duplex to be edited, e.g. 5′- and 3′-homology arms, and a gene editing sequence, and the said two sequences flanking the said gene editing sequence are optionally chemically modified, and the said 3′-homology arm comprises an optionally chemically modified RNA segment of 5 to 17 nucleotides complementary to 5′-end of the spacer of guide RNA, and is joined at its 5′-end by a oligonucleotide comprising said gene editing sequence and said 5′-homology arm as a template for DNA synthesis (i.e. FIGS. 4 and 5).

In some embodiments, the said conjugated oligonucleotide is a donor template which comprises two sequences overlapping with the target strand or with the non-target strand of the DNA duplex to be edited, e.g. 5′- and 3′-homology arms, and a gene editing sequence, and the said two sequences flanking the said gene editing sequence are optionally chemically modified, and the said 3′-homology arm comprises an optionally chemically modified DNA or chimeric oligonucleotide segment of 5 to 17 nucleotides complementary to 5′-end of the spacer of guide RNA, and is joined at its 5′-end by a oligonucleotide comprising said gene editing sequence and said 5′-homology arm as a template for DNA synthesis (i.e. FIGS. 4 and 5).

In some embodiments, the said conjugated oligonucleotide is a donor template for DNA repair or serves as a bridging molecule to a second oligonucleotide via complementary hybridization.

NON-NUCLEOTIDE LINKERS (nNt-linker)

An nNt-Linker, formed by chemical ligation, comprises an M core structure of Formula M-1 to M-13 as non-limiting examples:

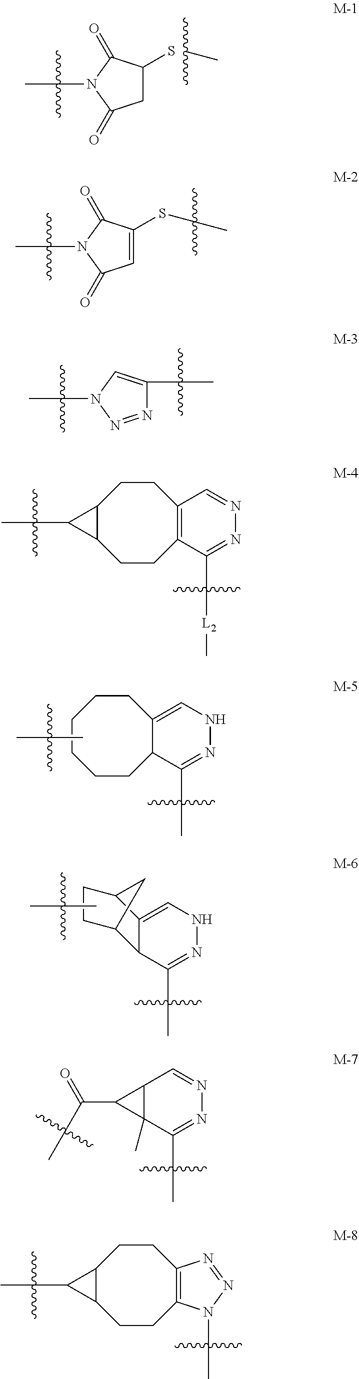

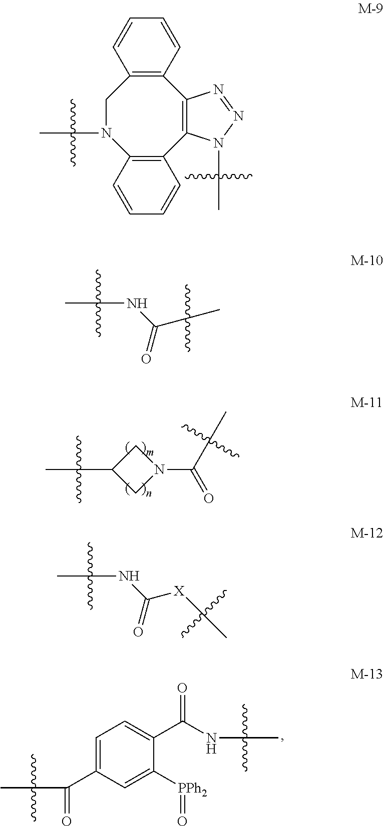

wherein X═O, S, NH, or CH₂, m=0 to 3 and n=0 to 3,
   and two L linkers comprising Formula L-1 to L-23 as non-limiting examples:

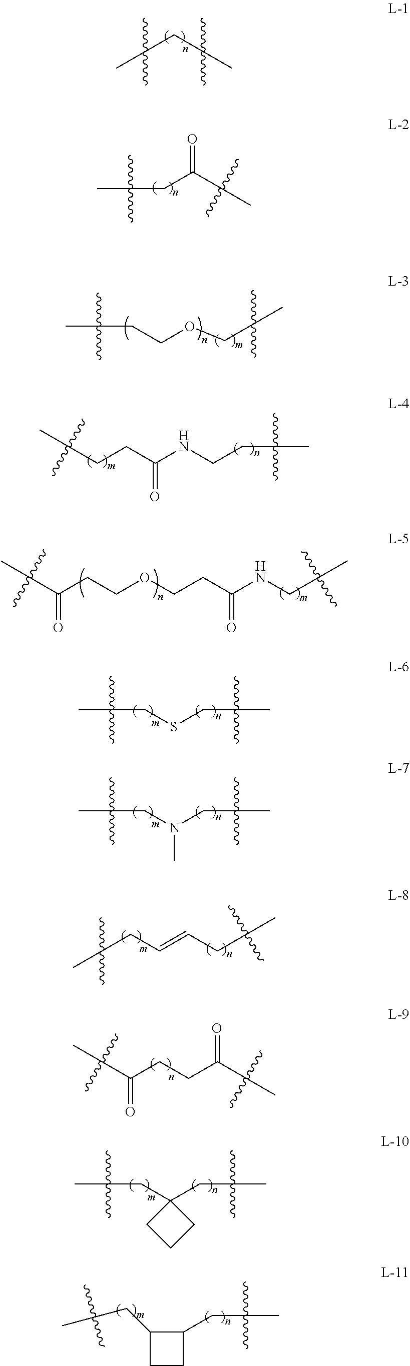

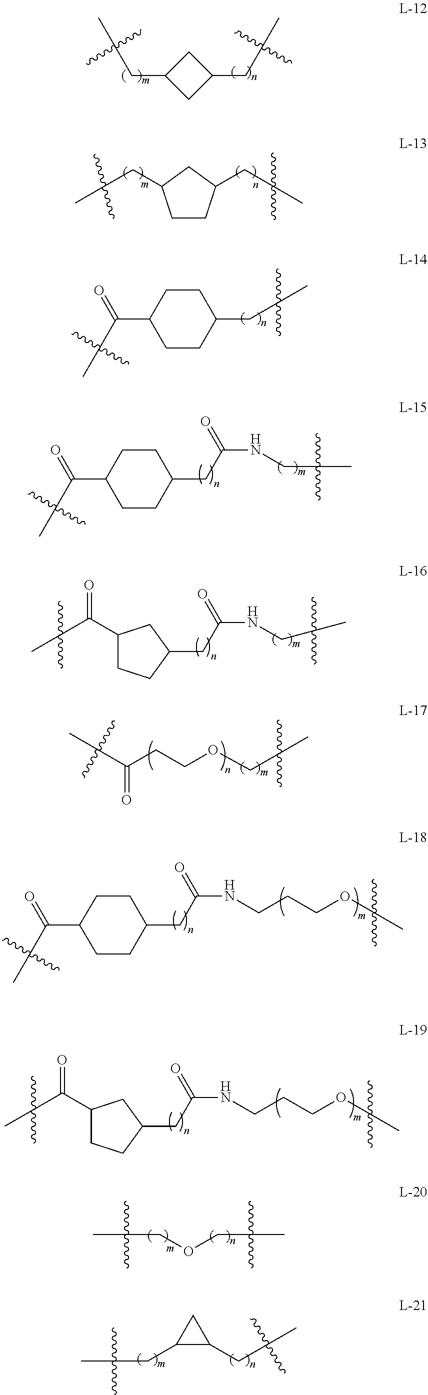

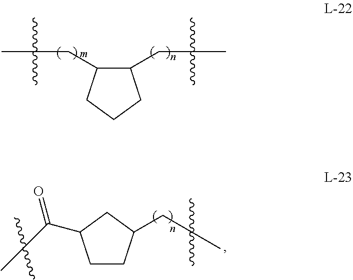

wherein m=0 to 16 and n=0 to 16,
   said L linkers and said M core structure are joined as L-M-L, wherein the two L linkers are the same or different, and each L optionally comprises one or more structures of Formula L-1 to L-23 or partial structure(s), and attached to two terminal nucleotides of Formula Nuc-1 to Nuc-18 as non-limiting examples:

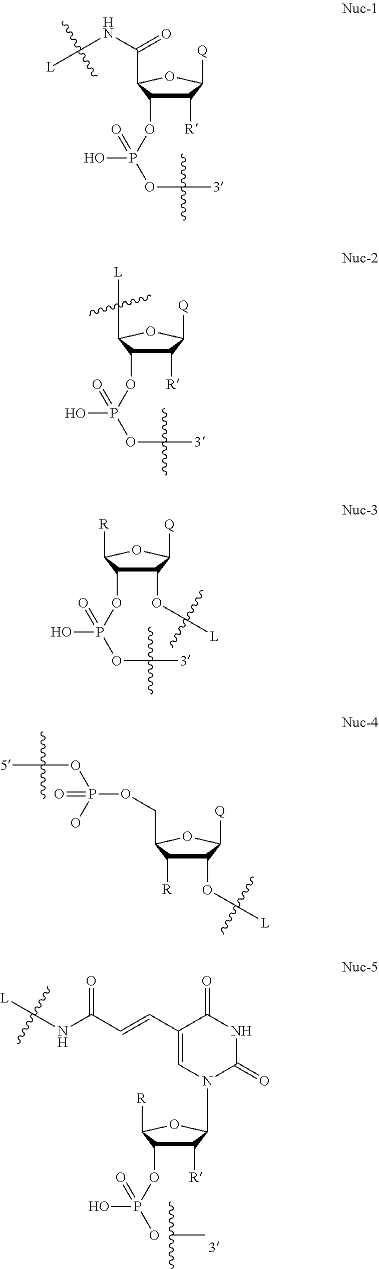

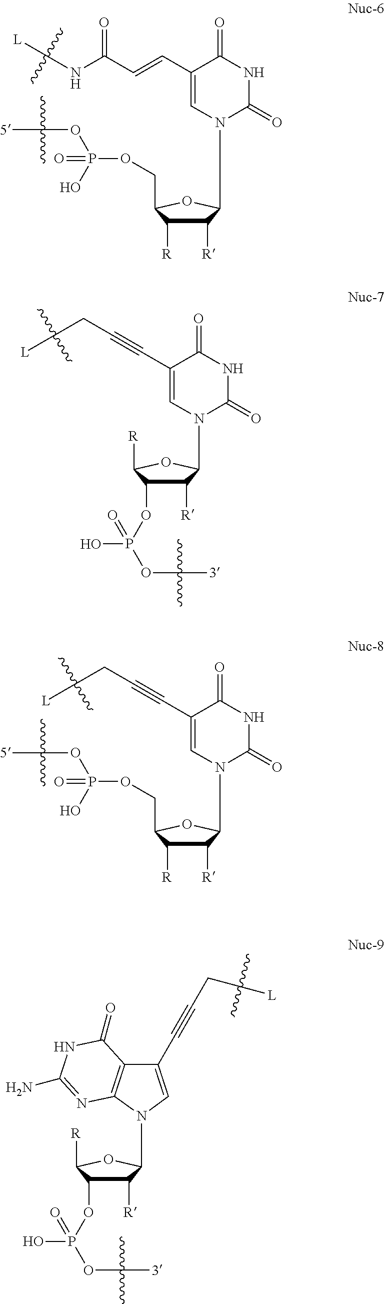

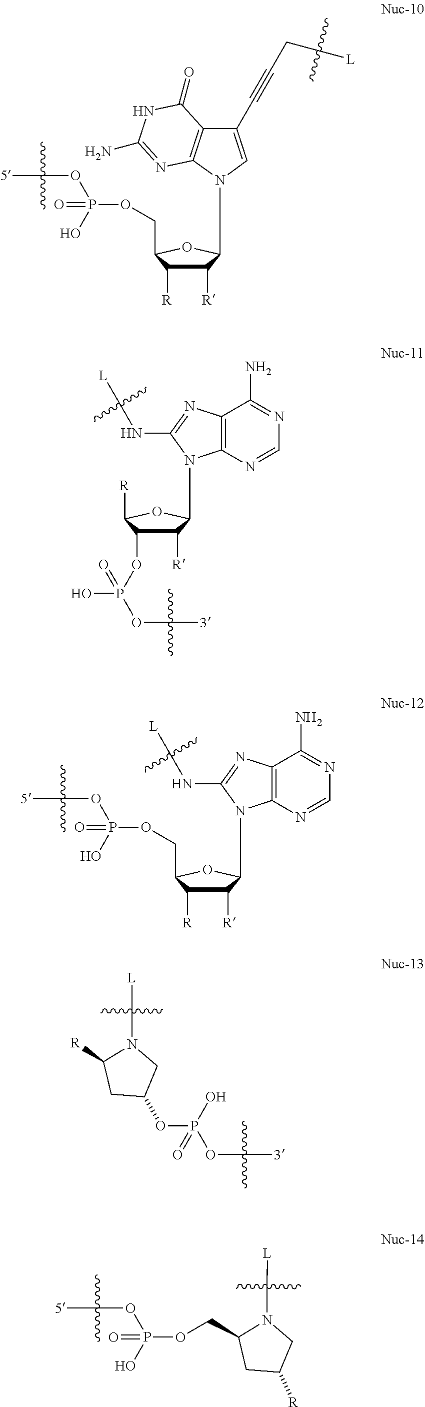

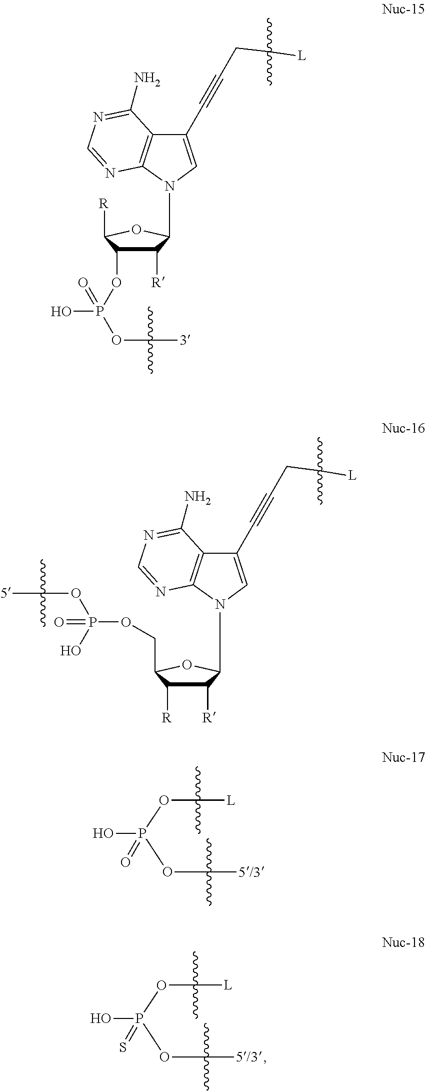

wherein the attached positions are

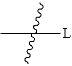

### to L-M-L and

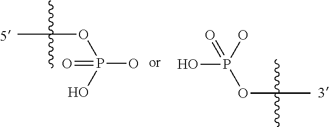 [SMILES: *OP(=O)(O)O ; *OP(=O)(O)O]

to upstream and downstream oligonucleotides, respectively, and wherein R is H, OH,

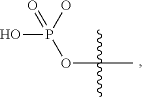 [SMILES: *OP(=O)(O)O]

### CH₂OH,

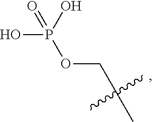 [SMILES: *COP(=O)(O)O]

F, NH₂, OMe, CH₂OMe, OCH₂CH₂OMe, an alkyl, a cycloalkyl, an aryl, or heteroaryl, R′ is H, OH

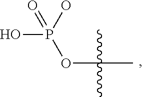 [SMILES: *OP(=O)(O)O]

### CH₂OH,

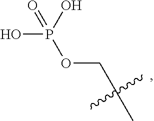 [SMILES: *COP(=O)(O)O]

F, NH₂, OMe, CH₂OMe, OCH₂CH₂OMe, an alkyl, a cycloalkyl, an aryl, or a heteroaryl, and Q is a natural or a non-natural nucleic acid base.

In some embodiments, the M core structure, L, and terminal nucleotides are optionally modified with substituents such as halogen (F, Cl, Br, I), lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, an optionally substituted aryl, an optionally substituted heteroaryl, an optionally substituted sulfonyl, or optionally substituted acyl, which includes but is not limited to C(═O) alkyl, NR′₂, CN, CO₂H, CO₂R′, CONH₂, CONHR′, CONR′₂, CH═CHCO₂H, or CH═CHCO₂R′, wherein R′ is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, an nNt-Linker joins the 3′-terminal nucleotide of a crRNA and the 5′-terminal nucleotide of a tracrRNA. In some embodiments, an nNt-Linker joins the 5′-terminal nucleotide of a crRNA and the 3′-terminal nucleotide of a tracrRNA. In some embodiments, an nNt-Linker joins two oligonucleotide segments of tracrRNA.

In some embodiments, one of the two L's in an nNt-linker (L-M-L) is covalently linked to guide RNA(s), and the other is covalently linked to a PEG polymers, a non-PEG polymer, a ligand for cellular receptors, a lipid, an oligonucleotide, an antibody, a polysaccharides or a peptide.

In some embodiments, one of the two L's in nNt-linkers (L-M-L) is covalently linked to an exposed amino acid residue of a Cas protein such as lysine, serine, and cysteines, and the other is covalently linked to a PEG polymers, a non-PEG polymer, a ligand for cellular receptors, a lipid, an oligonucleotide, an antibody, a polysaccharides or a peptide.

In some embodiments, the nNt-linkers between the two nucleotides/nucleosides are represented by the following formulas:

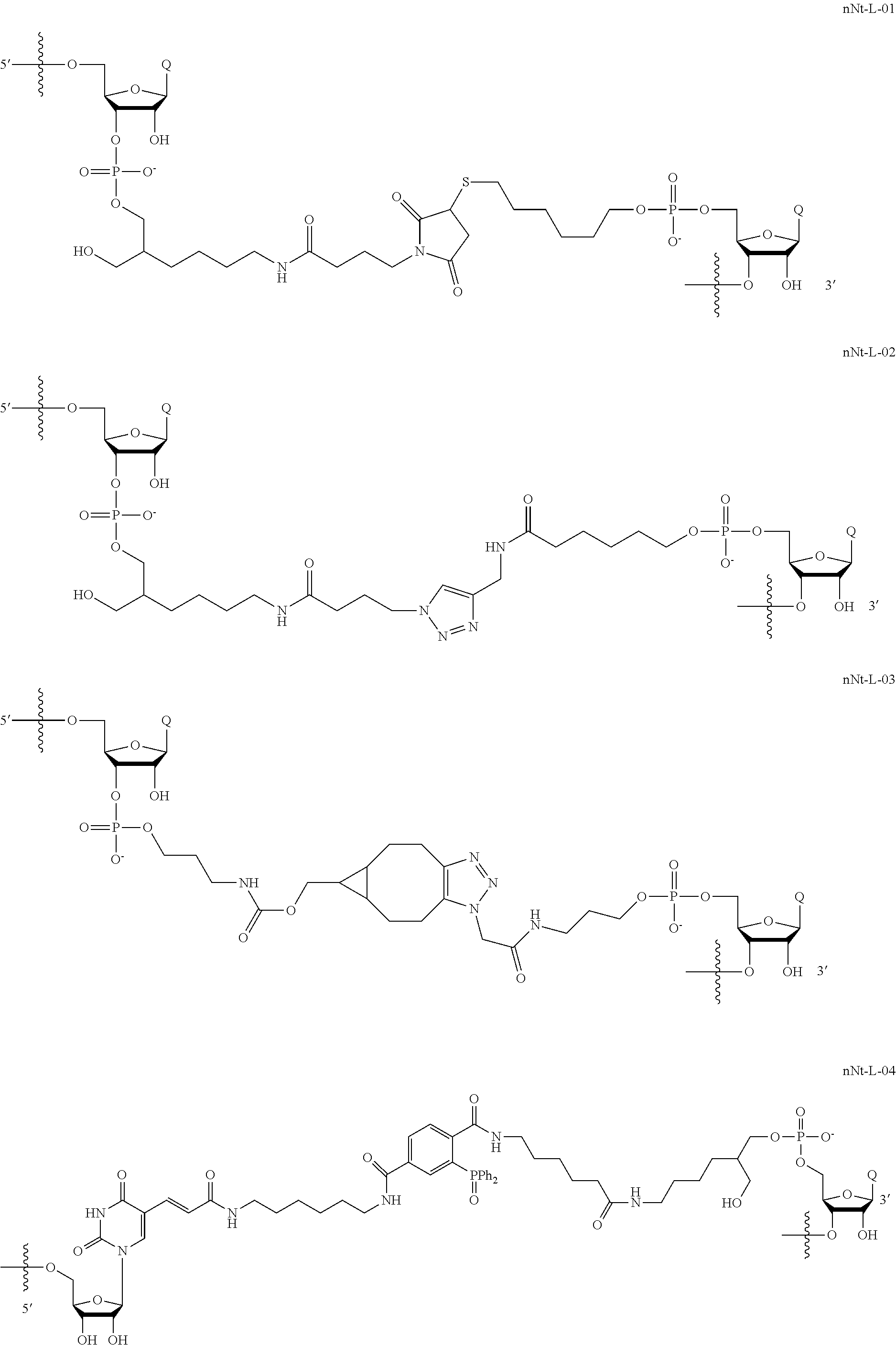

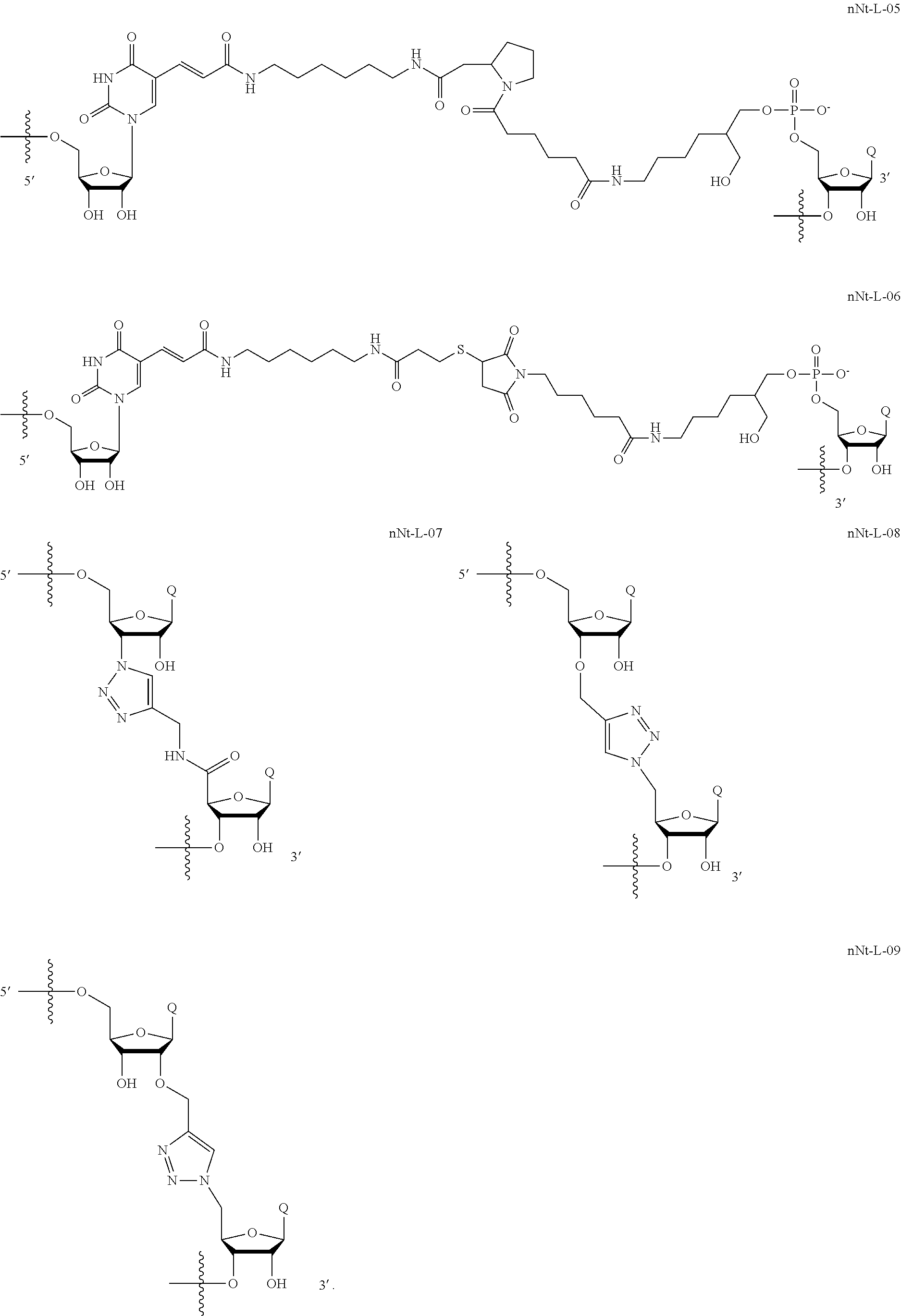

### Crispr Effector Proteins

In some embodiments, CRISPR effector endonuclease is selected from Cas proteins of Type II, Class 2 including Streptococcus pyogenes-derived Cas9 (SpCas9, 4.1 kb), smaller Cas9 orthologues, including Staphylococcus aureus-derived Cas9 (SaCas9, 3.16 kb), Campylobacter jejuni-derived Cas9 (CjCas9, 2.95 kb), Streptococcus thermophilus Cas9 (St1Cas9, 3.3 kb), Neisseria meningitidis (NmCas9, 3.2 kb), and many other variants of engineered Cas9 proteins such as SpCas9-HF1, eSpCas9, and HypaCas9, proteins of Type V, Class 2 including Cas12 (Cas12a (Cpf1), Cas12b (C2c1), Cas 12c, Cas12e, Cas12g, Cas12h, Cas12i, and etc.) and Cas14, and proteins of Type VI, Class 2 such as Cas13a and Cas13b. The said CRISPR effector protein can be a nickase e.g. SpCas9-nickase (D10A or H840A), or a catalytically inactive protein e.g. Cas9 (dCas9) coupled/fused with a protein effector such as FokI, transcription activator(s), transcription repressor(s), catalytic domains of DNA methyltransferase, histone acetyltransferase and deacetylase, reverse transcriptase (prime editor), and nucleic acid deaminases (base editor).

In another embodiment, the said CRISPR effector endonuclease is an artificial one comprising one or more functional domains derived from human.

In yet another embodiment, the said CRISPR effector endonuclease is a class 2 CRISPR Cas protein functionalized by site-directed mutagenesis to introduce orthogonal conjugating sites such as cysteines and remove deleterious conjugating sites (e.g. C80 in SpCas9), and corresponding RNP conjugates are prepared by selective conjugations such as PEGylation of cysteines by maleimide chemistry.

In yet another embodiment, the RNP complex conjugates are synthesized by reactions with crosslinking reagents, or by enzymes such as TGase which catalyzes amine transfer reaction between a primary amine at terminal side of PEG and the carboxamide of glutamine.

### Tissue Tropic Viral Vectors Encoding a Cas Protein or a Cas-Tracrrna Complex

In some embodiments, the said viral vector encoding a Cas effector endonuclease such as Cas9, or a Cas9-tracrRNA complex, is a retrovirus, lentivirus, adenovirus, AAV, or baculovirus.

In some embodiments, the said viral vector encoding a Cas effector endonuclease such as Cas9, or a Cas9-tracrRNA complex, is an engineered AAV or AAV chimera to enable high transduction efficiency at a targeted tissue by changing the tropism of AAV capsids and to have low immunogenicity by evading human preexisting anti-AAV capsid neutralizing antibodies.

In some embodiments, the said viral vector encoding a Cas effector endonuclease such as Cas9, or a Cas9-tracrRNA complex, is a native or an engineered AAV to enable brain tissue targeted delivery. Such AAV serotypes include as non-limiting examples AAV1, AAV2/DJ, AAV2/DJ8, AAV2g9, AAV2-retro and scAAV9.

In some embodiments, the said viral vector encoding a Cas effector endonuclease such as Cas9, or a Cas9-tracrRNA complex, is a native or an engineered AAV to enable liver tissue targeted delivery. Such AAV serotypes include as non-limiting examples AAV8 and AAV3.

In some embodiments, the said viral vector encoding a Cas effector endonuclease such as Cas9, or a Cas9-tracrRNA complex, is a native or an engineered AAV to enable muscle tissue targeted delivery. Such AAV serotypes include as non-limiting examples AAV6, AAV8 and AAV9.

In some embodiments, the said viral vector encoding a Cas effector endonuclease such as Cas9, or a Cas9-tracrRNA complex, is a native or an engineered AAV to enable heart tissue targeted delivery. Such AAV serotypes include as non-limiting examples AAV rh74 and AAV9.

In some embodiments, the said viral vector encoding a Cas effector endonuclease such as Cas9, or a Cas9-tracrRNA complex, is a native or an engineered AAV to enable retina tissue targeted delivery. Such AAV serotypes include as non-limiting examples AAV1, AAV2, AAV5, AAV8 and AAV9.

In some embodiments, the said viral vector encoding a Cas effector endonuclease such as Cas9, or a Cas9-tracrRNA complex, is a native or an engineered AAV to enable lung tissue targeted delivery. Such AAV serotypes include as non-limiting examples AAV9.

In some embodiments, the expression of a Cas effector endonuclease such as Cas9, or a Cas9-tracrRNA complex by the said viral vector, is driven by inducible tissue-specific promoters.

In some embodiments, the expression of a Cas effector endonuclease such as Cas9, or a Cas9-tracrRNA complex by the said viral vector, is driven by brain tissue-specific promoters such as pMecp2, hSyn1, TRE3G and EFS as non-limiting examples.

In some embodiments, the expression of a Cas effector endonuclease such as Cas9, or a Cas9-tracrRNA complex by the said viral vector, is driven by liver tissue-specific promoters such as TBG and HCRhAATp or by lung tissue-specific promoters such as EFS, as non-limiting examples.

In some embodiments, the expression of a Cas effector endonuclease such as Cas9, or a Cas9-tracrRNA complex by the said viral vector, is driven by heart tissue-specific promoters such as CMV, Myh6, CB and CK7-miniCMV as non-limiting examples.

In some embodiments, the expression of a Cas effector endonuclease such as Cas9, or a Cas9-tracrRNA complex by the said viral vector, is driven by retina tissue-specific promoters such as EFS, CMV, Spc512, pMecp2, and Picam2 as non-limiting examples.

In some embodiments, the expression of a Cas effector endonuclease such as Cas9, or a Cas9-tracrRNA complex by the said viral vector, is driven by muscle tissue-specific promoters such as CMV, EFS and CK8 as non-limiting examples.

In some embodiments, the said viral vector encoding a Cas effector endonuclease such as Cas9, or a Cas9-tracrRNA complex, is administrated locally or systematically to optimize tissue targeted deliveries in animals and human. The injection site is intravenous, subcutaneous, intramuscular, intradermal, intraperitoneal, intravitreal, intranasal, intratracheal, subretinal, intraocular, or intracardiac, or at other sites. Chemically modified lgRNA(s), sgRNA(s), crRNA(s), or their conjugates are administrated either in an aqueous solution gymnotically or with transfection reagents or in a non-viral carrier by co-injections or separate injections.

In some embodiments, the said viral vector encoding a Cas effector endonuclease such as Cas9, or a Cas9-tracrRNA complex, is administrated to isolated cells including T cells, and chemically modified lgRNA(s), or crRNA(s) are administrated either in an aqueous solution gymnotically by electroporation or microinjection, or with transfection reagents or in a non-viral carrier.

### Non-Viral Delivery of Rnp Complexes

In some embodiments, guide RNA conjugates, conjugates of guide RNA(s)-Cas protein (RNP) complexes such as PEGylated RNP complexes, or mixed lgRNA conjugate(s) and mRNA encoding a Cas-NLS protein are delivered with lipids well used in delivery of therapeutic nucleic acids such as DOPE, DOTAP, DOPS, DLin-MC3-DMA, cKK-E12, DSPC, POPE, 503013, PEG-DMG, bio-reducible lipid 8-O14B, cholesterol, and etc.

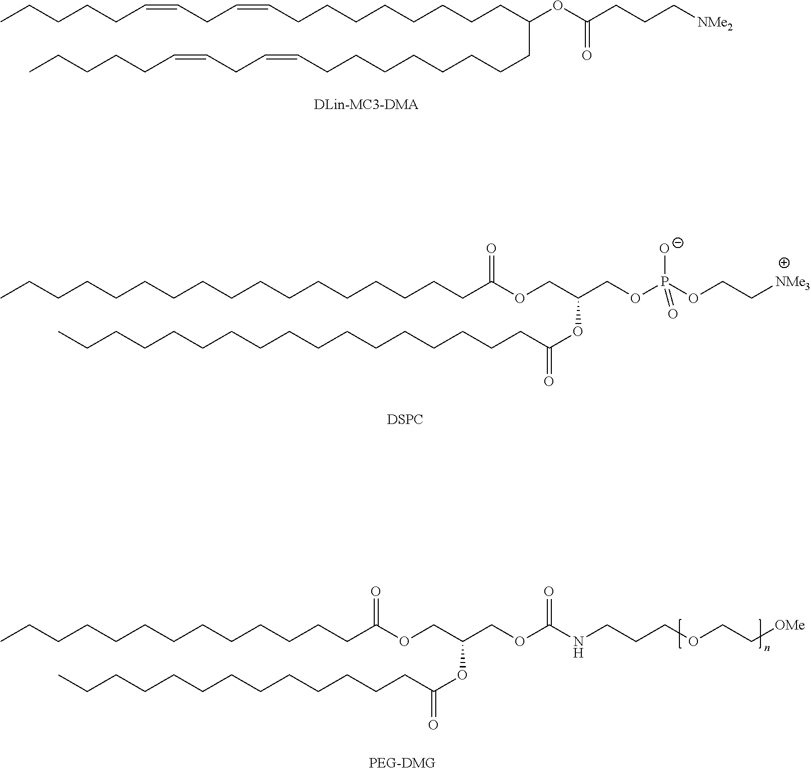

In other embodiments, conjugates of guide RNA(s)-Cas protein (RNP) complexes such as PEGylated RNP complexes are delivered within lipid nanoparticles, liposomes, or exosomes.

In yet another embodiment, conjugates of guide RNA(s) and mRNA encoding a Cas protein are delivered within lipid nanoparticles, liposomes, or exosomes.

In yet another embodiment, conjugates of guide RNA(s) are delivered with lipids or peptides, or within lipid nanoparticles, liposomes, or exosomes to cells stably or inducibly expressing a Cas protein, or with a viral vector encoding a Cas protein co-delivered.

In yet another embodiment, conjugates of crRNA(s) are delivered with lipids or peptides, or within lipid nanoparticles, liposomes, or exosomes to cells stably or inducibly expressing Cas9 protein and tracrRNA, or with a viral vector encoding a Cas9 protein and a tracrRNA co-delivered.

In some embodiments, the said crRNA(s), or lgRNAs, or sgRNAs, or their conjugates or conjugates of guide RNA(s)-Cas protein (RNP) complexes are administrated by electroporation or microinjection.

### Conjugates of Rnp Complexes

An aspect of the invention is directed to conjugates of guide RNA(s)-Cas protein (RNP) complexes such as a PEGylated CRISPR Cas protein-guide RNA complex(es), which are prepared by conjugation of a preformed guide RNA-CRISPR-Cas RNP(s) (RNP-I as an example),

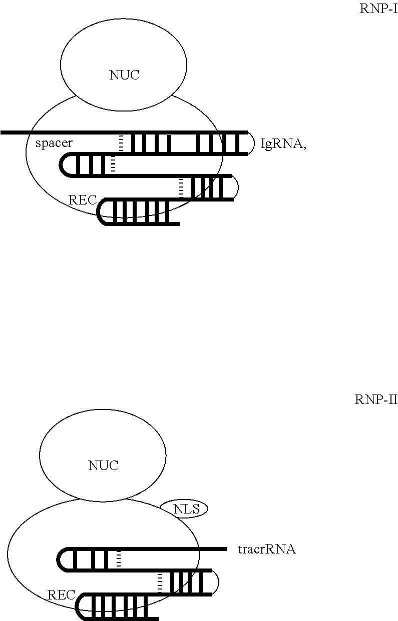

or prepared by conjugation of a Cas protein engineered for site-selective conjugations followed by complexing with a guide RNA(s) to form a RNP complex, wherein guide RNA(s) is an lgRNA composed of dual-modules (crRNA and tracrRNA) and comprising a single nNt-linker, dual or multiple nNt-linkers (lgRNA) formed by chemical ligation(s) and the ligation sites are preferably located at tetraloop (A32-U37) and/or stem 2 (C70-G79) (of reported sgRNA engineered for S. pyogenes Cas9), while any 3′, 5′-phosphodiester of sgRNA can be replaced by an nNt-Linker as a ligation site, a crRNA, an sgRNA, or dual guide RNAs, and wherein Cas protein is Cas9 comprising a nuclease lobe (NUC) and a recognition lobe (REC), an engineered Cas9, or a Cas protein other than the example of S. pyogenes Cas9 represented here; and wherein guide RNA(s) is chemically modified and optionally conjugated with one or more molecules selected from a group comprising PEG, non-PEG polymers, ligands for cellular receptors, lipids, oligonucleotides, antibodies, polysaccharides and peptides.

Another aspect of the invention is directed to CRISPR-Cas9-tracrRNA viral vectors or plasmids, encoding binary RNP complexes (RNP-II as an example) which further complex with crRNAs or their conjugates in targeted cells, wherein Cas9 comprising a nuclease lobe (NUC) and a recognition lobe (REC) can be a Cas protein other than the example of S. pyogenes Cas9 represented here, and can be an engineered Cas protein.

Another aspect of the invention is directed to a PEGylated CRISPR Cas protein-guide RNA complex(es), wherein the guide RNA(s) is a crRNA, dual guide RNAs, an sgRNA or an lgRNA.

Another aspect of the invention is directed to a CRISPR Cas protein conjugated with one or more molecules selected from a group comprising PEG, non-PEG polymers, ligands for cellular receptors, lipids, oligonucleotides, antibodies, polysaccharides and peptides.

Another aspect of the invention is directed to a conjugate of CRISPR Cas protein-guide RNA complex(es), wherein the guide RNA(s) is a conjugate of a crRNA, dual guide RNAs, an sgRNA or an lgRNA with one or more single strand DNAs (ssDNA) as a donor template for gene editing.

Another aspect of the invention is directed to a conjugate of CRISPR Cas protein-guide RNA complex(es), wherein the guide RNA(s) is a conjugate of a crRNA, dual guide RNAs, an sgRNA or an lgRNA with one or more single strand DNAs as a donor template for gene editing, and the CRISPR Cas protein is conjugated with one or more molecules selected from a group comprising PEG, non-PEG polymers, ligands for cellular receptors, lipids, oligonucleotides, antibodies, polysaccharides and peptides.

Some non-limiting examples of PEGylating reagents are given below.

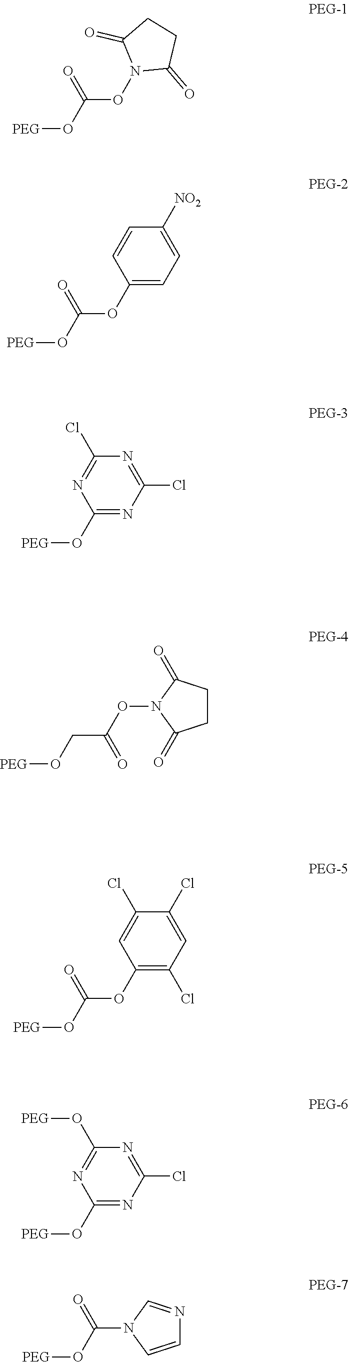

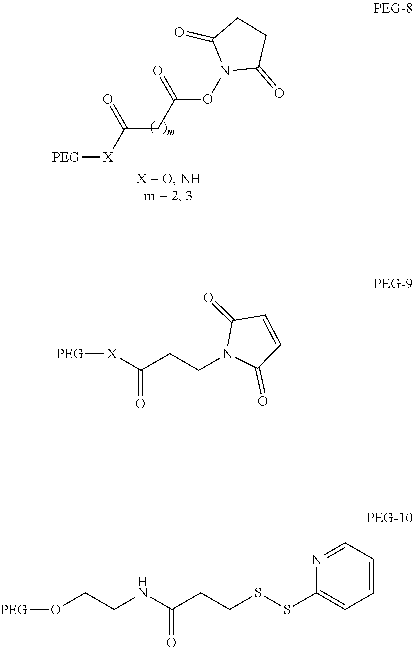

The PEG can be either linear or branched and conjugated at a lysine or cysteine residue:

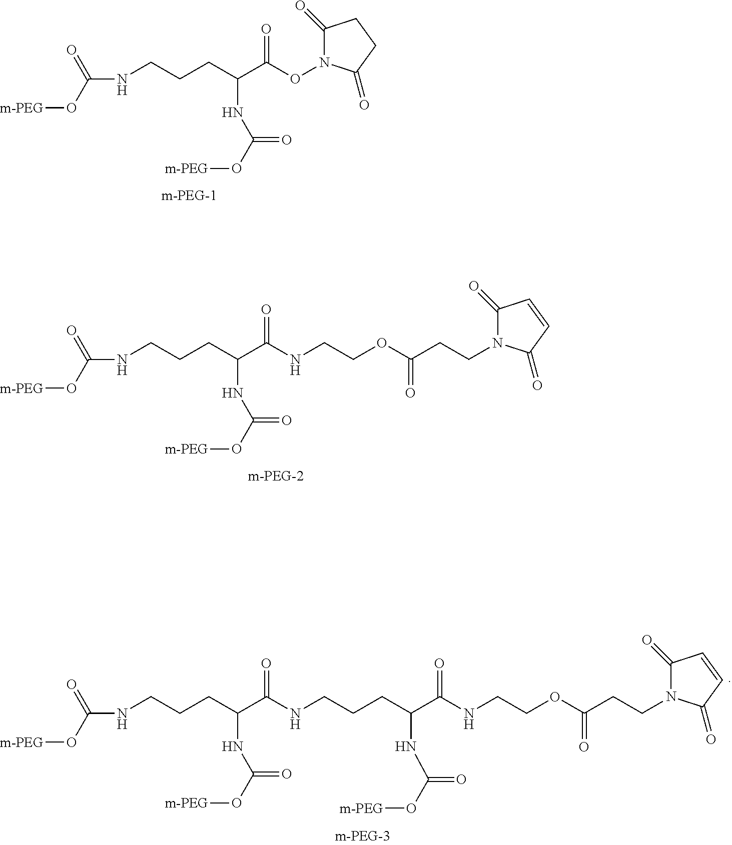

Another aspect of the invention is directed to a lipid conjugated Cas protein-guide RNA complex, which is prepared by lipid conjugation of a preformed CRISPR-Cas RNP complex or by lipid conjugation of a Cas protein engineered for site-selective conjugations followed by complexing with a guide RNA to form an RNP complex.

Some non-limiting examples of lipid conjugating reagents are given below:

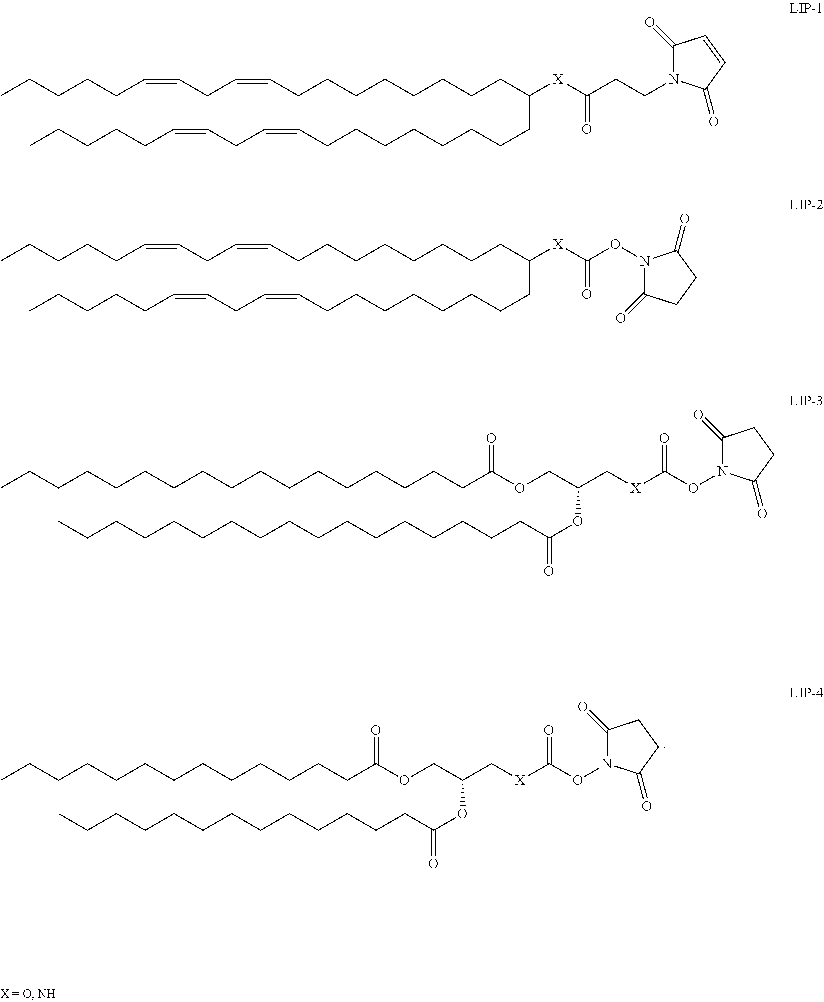

Another aspect of the invention is directed to a peptide conjugated Cas protein-guide RNA complex, which is prepared by peptide conjugation of a preformed CRISPR-Cas RNP complex or by peptide conjugation of a Cas protein engineered for site-selective conjugations followed by complexing with a guide RNA to form an RNP complex. Non-limiting examples include conjugates of short peptides of nuclear localization signal (NLS).

Another aspect of the invention is directed to a Cas protein-guide RNA complex conjugated with small molecule ligands of cell receptors, which are prepared by ligand conjugation of a preformed CRISPR-Cas RNP complex or by ligand conjugation of a Cas protein engineered for site-selective conjugations followed by complexing with a guide RNA to form a RNP complex. Non-limiting examples include asialoglycoprotein receptor ligands (ASGPrL) such as N-acetylgalactosamine (GalNAc) and lactobionic acid.

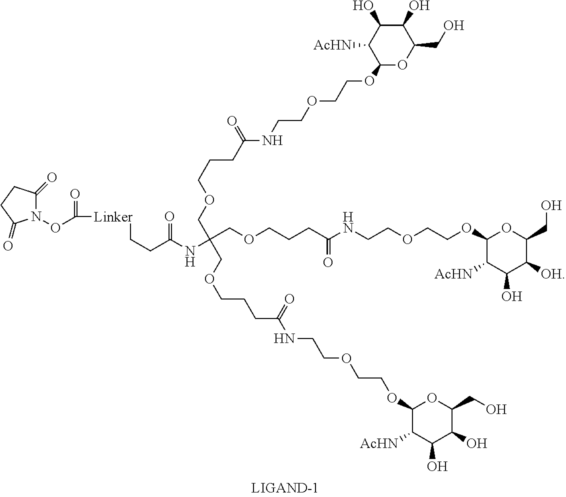

Yet another aspect of the invention is directed to a carbohydrate conjugated Cas protein-guide RNA complex, which is prepared by carbohydrate conjugation of a preformed CRISPR-Cas RNP complex or by carbohydrate conjugation of a Cas protein engineered for site-selective conjugations followed by complexing with a guide RNA to form an RNP complex. Non-limiting examples include oligosaccharides and polysaccharides.

Yet another aspect of the invention is directed to conjugates of an engineered Cas9 protein mutated at C80 and incorporating one or more cysteines for site-selective conjugations by site-directed mutagenesis.

The present invention relates to compositions of conjugates of a guide RNA(s)-Cas protein (RNP) complex and their uses as medicinal agents in treatment of viral infectious diseases and as gene regulation, disruption and/or correction-based therapeutics. The conjugate(s) comprises a guide RNA-Cas protein (RNP) complex and one or more molecules selected from a group comprising PEG, non-PEG polymers, ligands for cellular receptors, lipids, oligonucleotides, antibodies, polysaccharides, glycans and peptides and chemically linked to Cas protein and/or guide RNA(s).

The said uses may optionally include delivering an HDR template, wherein the template provides expression of a normal or less aberrant form of the protein to alleviate or eliminate a condition or disease state. The said HDR template may optionally be conjugated to guide RNA(s) and/or Cas protein.

Some aspects of the invention are directed to a donor template containing a “gene-editing-sequence” comprising one or more gene regulatory sequences or stop codons, and in some embodiments, the donor template(s) is conjugated to guide RNA(s) as part(s) of an lgRNA.

Some aspects of the invention are directed to PEGylated CRISPR-Cas9-lgRNA-conjugate and transfection reagent(s) systems.

Some aspects of the invention are directed to the use of conjugates of a guide RNA(s)-Cas protein (RNP) complex such as a PEGylated CRISPR-Cas9-lgRNA-conjugate system for antiviral therapy targeting against proviral DNAs or integrated viral DNA or episomal circular DNAs.

In some embodiments, non-limiting examples of targeted viral genomic sequences of HBV include:

(SEQ ID NO: 41)
ctctgctagatcccagagtg [aGG],
(SEQ ID NO: 42)
gctatcgctggatgtgtctg [cGG],
(SEQ ID NO: 43)
tggacttctctcaattttct [a[G[G]G]G]],
(SEQ ID NO: 44)
gggggatcacccgtgtgtct [tGG],
(SEQ ID NO: 45)
tatgtggatgatgtggtactgg [gGG],
(SEQ ID NO: 46)
cctcaccatacagcactc [gGG],
(SEQ ID NO: 47)
gtgttggggtgagttgatgaatc [tGG],

wherein nucleotides in [ ] are PAMs, or any sequence of 17-20 nt immediately next to a PAM sequence.

In some embodiments, the therapeutic Cas9-lgRNA RNP conjugates comprise multiple lgRNAs targeting at different sites in viral genome (multiplex editing).

In some embodiments, multiple crgRNAs, including but not limited to sequences of YMDD and its mutations at catalytic domain of HBV polymerase, corresponding to

(SEQ ID NO: 48)
tatgtggatgat gtggtactgg [gGG],
(SEQ ID NO: 49)
tatatggatgat gtggtattgg [gGG],
(SEQ ID NO: 50)
tatgtggatgat gtggtattgg [gGG],
(SEQ ID NO: 51)
tatatagatgat gtggtactgg [gGG],

are ligated to tracrgRNA to result in a mixture of lgRNAs, and thus a mixture of Cas9-lgRNA RNP conjugates to target drug resistance in therapies based on direct-acting antiviral agents (DAA).

In some embodiments, non-limiting examples of targeted viral genomic sequences of HIV include:

(SEQ ID NO: 52)
gattggcaga actacacacc [aGG],
(SEQ ID NO: 53)
atcagatatc cactgacctt [tGG],
(SEQ ID NO: 54)
gcgtggcctg ggcgggactg [gGG],
(SEQ ID NO: 55)
cagcagttct tgaagtactc [cGG],

wherein nucleotides in [ ] are PAMs, or any sequence of 17-20 nt immediately next to a PAM sequence.

In some embodiments, non-limiting examples of targeted viral genomic sequences of herpesviridae virus such as HSV and EBV include:

(SEQ ID NO: 56)
gccctggaccaacccggccc [gGG],
(SEQ ID NO: 57)
ggccgctgccccgctccggg [tGG],
(SEQ ID NO: 58)
ggaagacaatgtgccgcca [tGG],
(SEQ ID NO: 59)
tctggaccagaaggctccgg [cGG],
(SEQ ID NO: 60)
gctgccgcggagggtgatga [cGG],
(SEQ ID NO: 61)
ggtggcccaccgggtccgct [gGG],
(SEQ ID NO: 62)
gtcctcgagggggccgtcgc [gGG],

wherein nucleotides in [ ] are PAMs, or any sequence of 17-20 nt immediately next to a PAM sequence.

In some embodiments, non-limiting examples of targeted genomic sequences include 17-20 nt of genes encoding endogenous T-cell receptors (TCR), HLA class I (HLA-I) or T-cell inhibitory receptors or signal molecules such as programmed cell death protein 1 (PD1) and cytotoxic T lymphocyte-associate protein 4 (CTLA4), immediately next to a PAM sequence.

Other aspects of the invention are directed to the use of said CRISPR-Cas-guide RNA conjugate systems for gene therapy and treatments of human infectious diseases.

In some embodiments, targeted sequences are human genomic sequences incorporating disease causing mutations, e.g.

(SEQ ID NO: 63)
5′-aaa gaa aat atm mmt ggt gtt-3′,

wherein m is a mutation, and the “gene-editing-sequence” comprises

(SEQ ID NO: 64)
5′-aaa gaa aat atc ttt ggt gtt-3′.

Non-limiting examples of such human single-gene disorders are given below (Table 1). Other examples include human polygenic disorders such as heart disease and diabetes.

TABLE 1
Examples single-gene disorders and their prevalence

| | |
|---|---|
| | Prevalence |
| Disorder | (approximate) |

Autosomal dominant

| | |
|---|---|
| Familial hypercholesterolemia | 1 in 500 |
| Polycystic kidney disease | 1 in 1250 |
| Neurofibromatosis type I | 1 in 2,500 |
| Hereditary spherocytosis | 1 in 5,000 |
| Marfan syndrome | 1 in 4,000 |
| Huntington's disease | 1 in 15,000 |

Autosomal recessive

| | |
|---|---|
| Sickle cell anaemia | 1 in 625 |
| Cystic fibrosis | 1 in 2,000 |
| Tay-Sachs disease | 1 in 3,000 |
| Phenylketonuria | 1 in 12,000 |
| Mucopolysaccharidoses | 1 in 25,000 |
| Lysosomal acid lipase deficiency | 1 in 40,000 |
| Glycogen storage diseases | 1 in 50,000 |

X-linked

| | |
|---|---|
| Duchenne muscular dystrophy | 1 in 7,000 |
| Hemophilia | 1 in 10,000 |

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates that may need to be independently confirmed.

This disclosure is not limited to particular embodiments described, as such may vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### EXAMPLES

The following examples further illustrate embodiments of the disclosed invention, which are not limited by these examples.

### Example 1

### Synthesis of SaCas9 crRNA-GalNAc conjugate

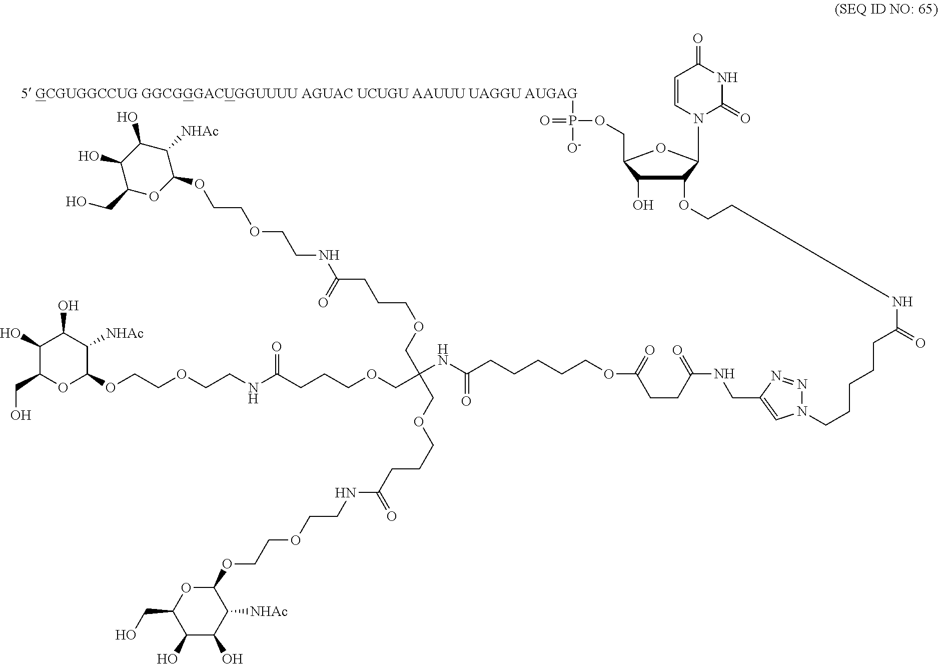

STEP 1. The 3′-amino-crRNA is prepared using 2′-TBS protected RNA phosphoramidite monomers with t-butylphenoxyacetyl protection of the A, G and C nucleobases and unprotected uracil. 0.3 M Benzylthiotetrazole in acetonitrile (Link Technologies) is used as the coupling agent, t-butylphenoxyacetic anhydride as the capping agent and 0.1 M iodine as the oxidizing agent. Oligonucleotide synthesis is carried out on an Applied Biosystems 394 automated DNA/RNA synthesizer using the standard 1.0 mole RNA phosphoramidite cycle. Uridine on CPG is prepared as before (US20160215275A1) and packed into a twist column. All β-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >97.5%.

Fmoc is then cleaved by treatment with 20% piperidine in DMF. The resulting 3′-end aminoethyl oligonucleotide is then treated with NHS ester of 6-azido caproic acid in DMF.

Cleavage of oligonucleotides from the solid support and deprotection are achieved by exposure to concentrated aqueous ammonia/ethanol (3/1 v/v) for 2 h at room temperature followed by heating in a sealed tube for 45 min at 55° C. and desilylation in 1.0 M TBAF in THF for 24 h to give 3′-azide-crRNA.

Alternatively, the step of reaction with NHS ester is skipped, and the above resulting fully deprotected oligonucleotide is dissolved in 0.5 M Na₂CO₃/NaHCO₃ buffer (pH 8.75) and incubated with succinimidyl-6-azidohexanate (20 eq.) in DMSO to give 3′-azide-crRNA.

STEP 2. To a solution of tri-GalNAc-alkyne and 3′-azide-crRNA (0.2 nmol of each) in 0.2 M NaCl (50 μL) at room temperature are added tris-hydroxypropyl triazole ligand (28 nmol in 42 μL 0.2 M NaCl), sodium ascorbate (40 nmol in 4 μL 0.2 M NaCl) and CuSO₄·5H₂O (4 nmol in 4.0 μL 0.2 M NaCl), under argon. The reaction mixture is kept under argon at room temperature for the desired time, and formamide (50 μL) is added. The reaction is analyzed by loading directly onto a 20% polyacrylamide electrophoresis gel, and purified by reversed-phase HPLC to give the crRNA-tri-GalNAc conjugate.

### Example 2: Cas9::crRNA-GalNAc::tracrRNA Complex

### Cas9 Protein:

Recombinant Cas9 protein is available from New England BioLabs, Inc. and other providers or is expressed and purified from E. coli by a routinely used protocol (Anders, C. and Jinek, M. Methods Enzymol. 2014, 546, 1-20). The purity and concentration of Cas9 protein are analyzed by SDS-PAGE.

Cas9::crRNA-GalNAc::tracrRNA Complex:

tracrRNA is synthesized on an Applied Biosystems 394 automated DNA/RNA synthesizer as reported. Cas9, crRNA-GalNAc and tracrRNA are preincubated in a 1:5:5 molar ratio in the cleavage buffer to reconstitute the Cas9::crRNA-GalNAc::tracrRNA (RNP) complex.

Alternatively, tracrRNA is prepared by in vitro transcription at the presence of a RNA polymerase such as bacteriophage T7 RNA polymerase.

### Example 3: PEGylation of CRISPR-Cas RNP Complex

The RNP (guide RNA-Cas protein) PEGylation is performed at different concentrations of m-PEG-pNP or m-PEG-NHS. RNP at 2 mg/mL in saline is incubated with varying concentrations of m-PEG-pNP or m-PEG-NHS (0.5, 1, 2, 4 and 6 mg/mL) in 1.5-mL microfuge tubes. The reaction is allowed to proceed at 30° C. for 3 hours. The PEGylated RNP complex(es) is used for in vitro DNA cleavages according to reported procedures without further purification.

Alternatively, before its storage at −80° C. or uses, size exclusion chromatography (SEC) of PEGylated RNP complex(es) is performed on a Akta Purifier using a HiLoad 16/60 S200 superdex column with gel filtration buffer (20 mM HEPES pH 7.5, 150 mM KCl, 10% (v/v) glycerol) with a flow rate of 1 mL/min. PEGylated RNP complex(es) is loaded in volumes no greater than 1 mL.

### Example 4: Expression and Purification of SpCas9 (Cys-SpCas9: M1C, C80A, S145C, S204C, S469C, S1159C)

SpCas9 expression plasmid containing amino acid substitutions are generated by standard PCR and molecular cloning.

The primers are synthesized on an Applied Biosystems 394 automated DNA/RNA synthesizer using the standard 1.0 mole DNA phosphoramidite cycle. Nucleoside on CPG support is packed into a twist column. All β-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >99%.

The Cys-SpCas9 is expressed in E. coli strain Rosetta 2 DE3 cells, and the protein is isolated and purified as described (Anders, et al. Methods Enzymol. 2014, 546, 1-20).

### Example 5: PEGylation of Cys-SpCas9 Protein

PEGylation of Cys-SpCas9 protein is performed at different concentrations of m-PEG-Maleimide. Protein at 2 mg/mL in saline is incubated with varying concentrations of m-PEG-Maleimide (0.5, 1, 2, 4 and 6 mg/mL) in 1.5-mL microfuge tubes. The reaction is allowed to proceed at 30° C. for 3 hours.

Before its storage at −80° C. or uses, size exclusion chromatography (SEC) of PEGylated RNP complex(es) is performed on a Akta Purifier using a HiLoad 16/60 S200 superdex column with gel filtration buffer (20 mM HEPES pH 7.5, 150 mM KCl, 10% (v/v) glycerol) with a flow rate of 1 mL/min. PEGylated RNP complex(es) is loaded in volumes no greater than 1 mL.

### Example 6: Cys-SpCas9::lgRNA Complex

The PEGylated Cys-SpCas9 protein and lgRNA are preincubated in a 1:2 molar ratio in the cleavage buffer to reconstitute the Cas::lgRNA complex. The PEGylated RNP complex(es) is used for in vitro DNA cleavages according to reported procedures without further purification.

### Example 7: ON-1 (SEQ ID NO: 66)

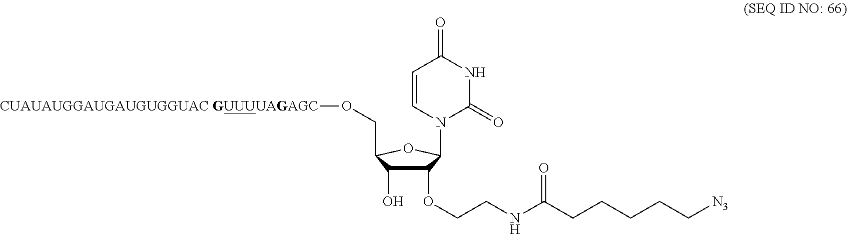

ON-1 is synthesized in a way similar to the synthesis of 3′-azide-crRNA in EXAMPLE 1, except the oligonucleotide is truncated at its 3′-end. The oligonucleotide is cleaved off the solid phase and fully deprotected, and purified as described in EXAMPLE 1.

### Example 8: ON-2

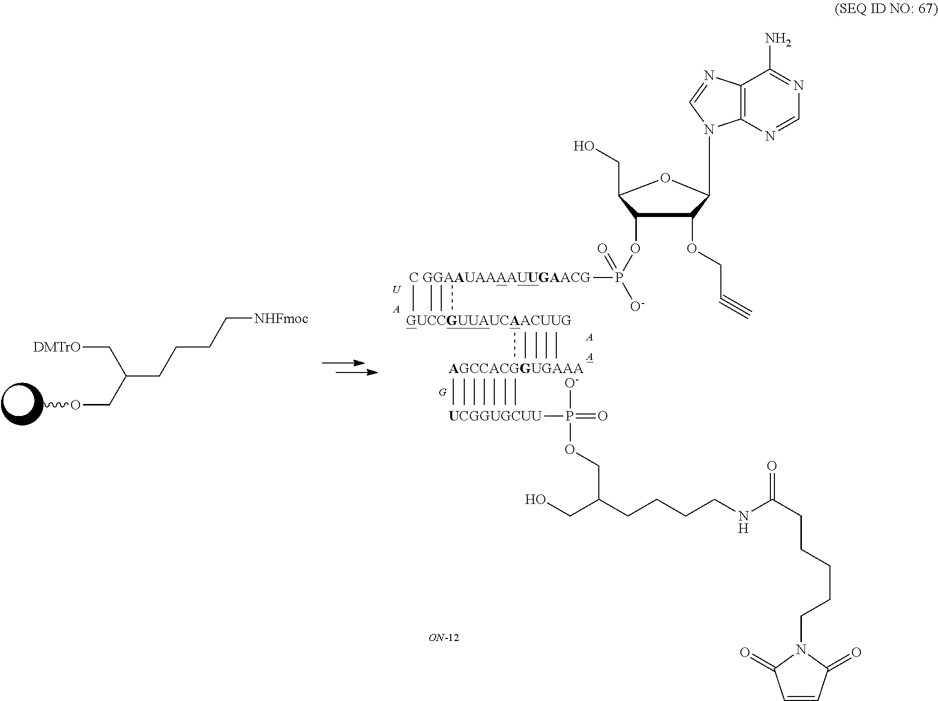

ON-2 is synthesized in a way similar to the synthesis of ON-1, except that a solid phase support linked to Fmoc-hexylamine is used, and 2′-propargyl adenosine phosphoramidite is used to introduce an alkyne.

Fmoc is then cleaved by treatment with 20% piperidine in DMF. The resulting 3′-end aminohexyl oligonucleotide is then treated with NHS ester of 6-maleimide caproic acid in DMF.

Cleavage of oligonucleotides from the solid support and full deprotection are achieved by exposure to concentrated aqueous ammonia/ethanol (3/1 v/v) for 2 h at room temperature followed by heating in a sealed tube for 45 min at 55° C. and desilylation in 1.0 M TBAF in THF for 24 h.

Alternatively, the step of reaction with NHS ester is skipped, and the above resulting fully deprotected oligonucleotide is dissolved in 0.5 M Na₂CO₃/NaHCO₃ buffer (pH 8.75) and incubated with NHS ester of 6-maleimide caproic acid (20 eq.) in DMSO to give ON-2.

### Example 9: ON-3 (SEQ ID NO: 68)

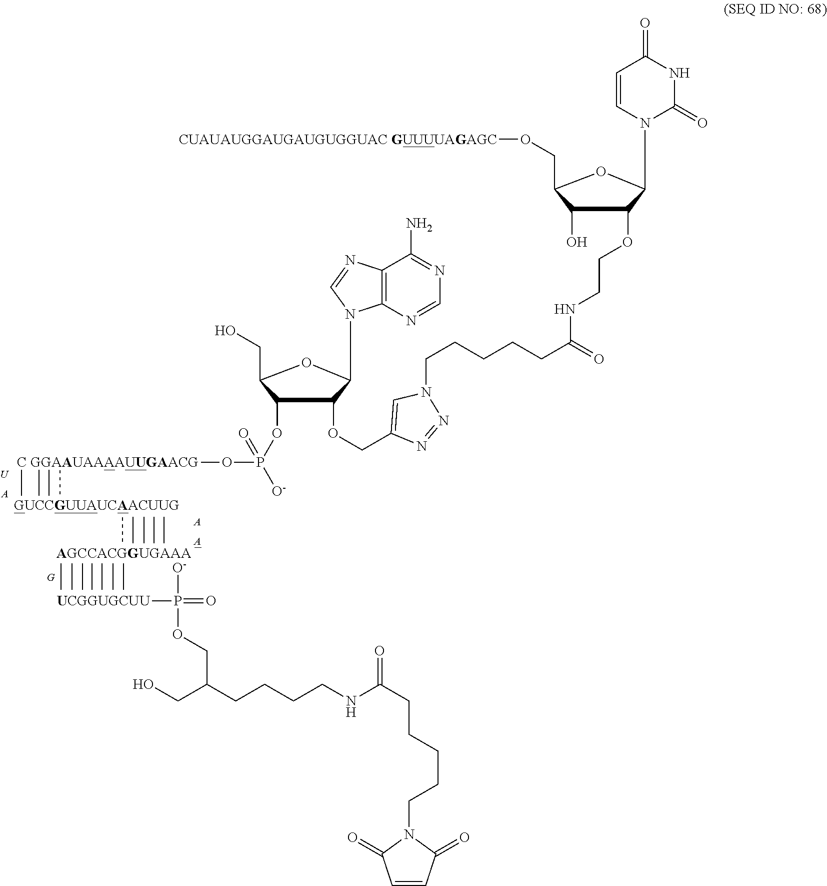

A solution of alkyne ON-1 and azide ON-2 (0.2 nmol of each) in 0.2 M NaCl (50 μL) is annealed for 30 min at room temperature. In the meantime, tris-hydroxypropyl triazole ligand (28 nmol in 42 μL 0.2 M NaCl), sodium ascorbate (40 nmol in 4 μL 0.2 M NaCl) and CuSO₄·5H₂O (4 nmol in 4.0 μL 0.2 M NaCl), are added under argon. The reaction mixture is kept under argon at room temperature for the desired time, and formamide (50 μL) is added. The reaction is analyzed by loading directly onto a 20% polyacrylamide electrophoresis gel, and purified by reversed-phase HPLC.

### Example 10: ON-4 (SEQ ID NO: 69)

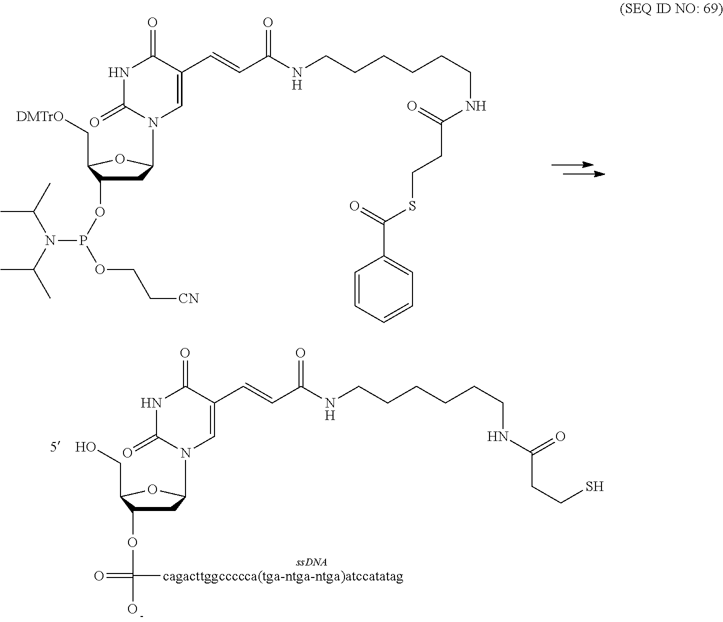

ON-4 is synthesized in a way similar to the synthesis of primers in Example 4.

Cleavage of oligonucleotides from the solid support and deprotection are achieved by exposure to concentrated aqueous ammonia/ethanol (3/1 v/v) for 2 h at room temperature followed by heating in a sealed tube for 45 min at 55° C.

### Example 11: lgRNA-ssDNA (SEQ ID NO: 70) and sgRNA-ssDNA (SEQ ID NO: 72)

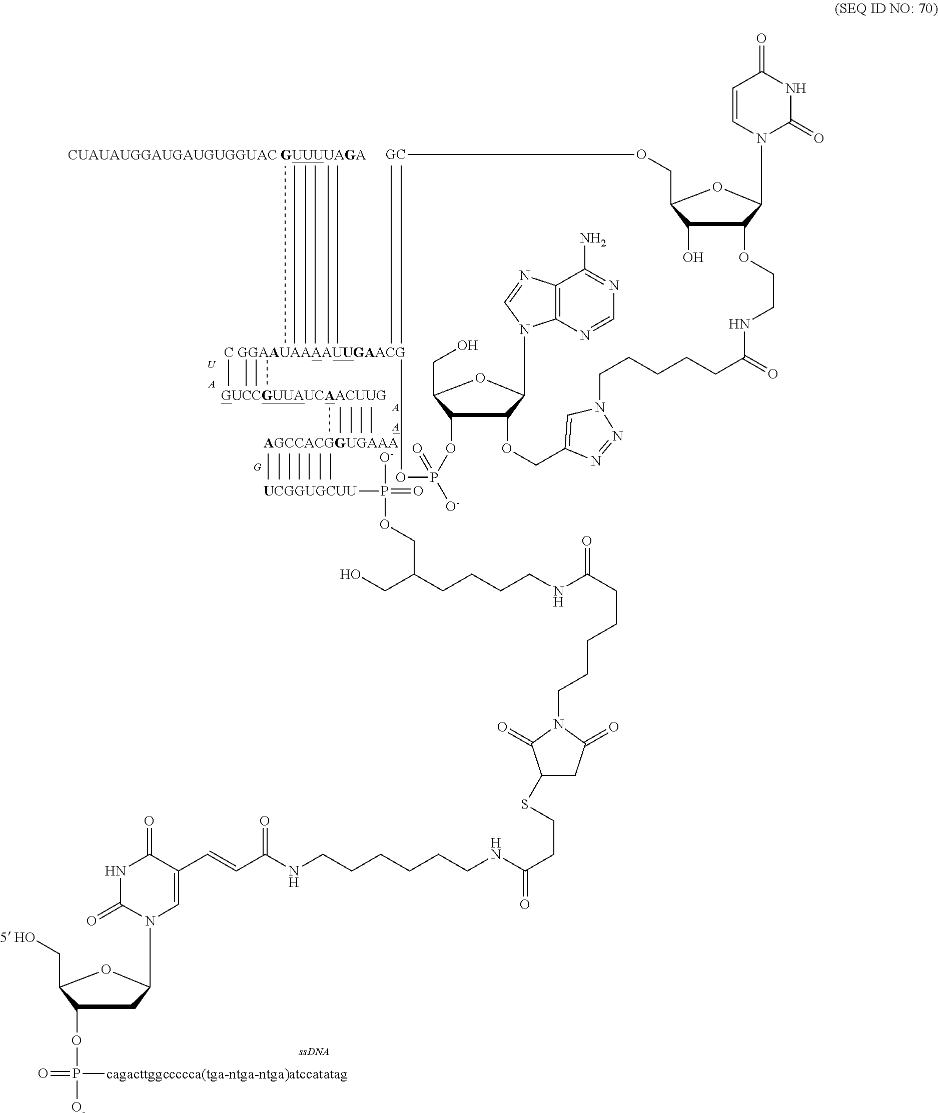

The ON-3 oligonucleotide carrying a maleimido group is incubated with 5′-SH-oligonucleotide (ON-4, 1:1 molar ratio) in 0.1 M triethylammonium acetate (TEAA) at pH 7.0 overnight at room temperature. The reaction mixture is analyzed and separated by HPLC to give the lgRNA-ssDNA.

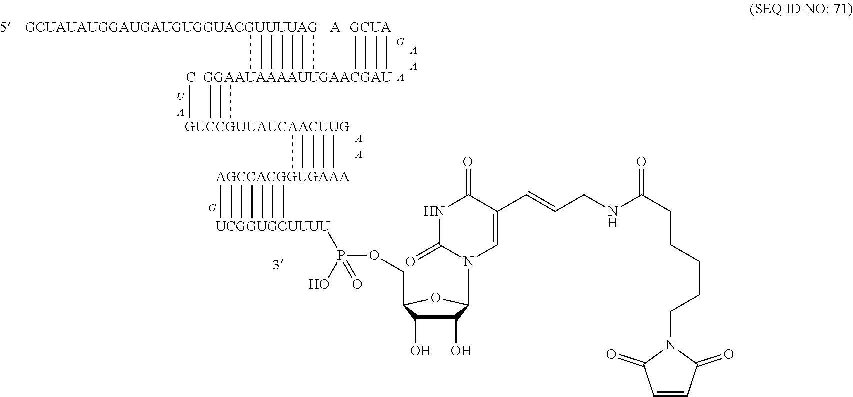

Alternatively, ON-3 oligonucleotide is substituted with an sgRNA carrying a maleimido group (ON-5 (SEQ ID NO: 71)) prepared from an sgRNA (incorporated an amine at its 3′-end) by in vitro transcription at the presence of a RNA polymerase such as bacteriophage T7 RNA polymerase, followed by reacting the amine to introduce a maleimido group via NHS chemistry. The said amine at the 3′-end is incorporated from a uridine triphosphate analogue.

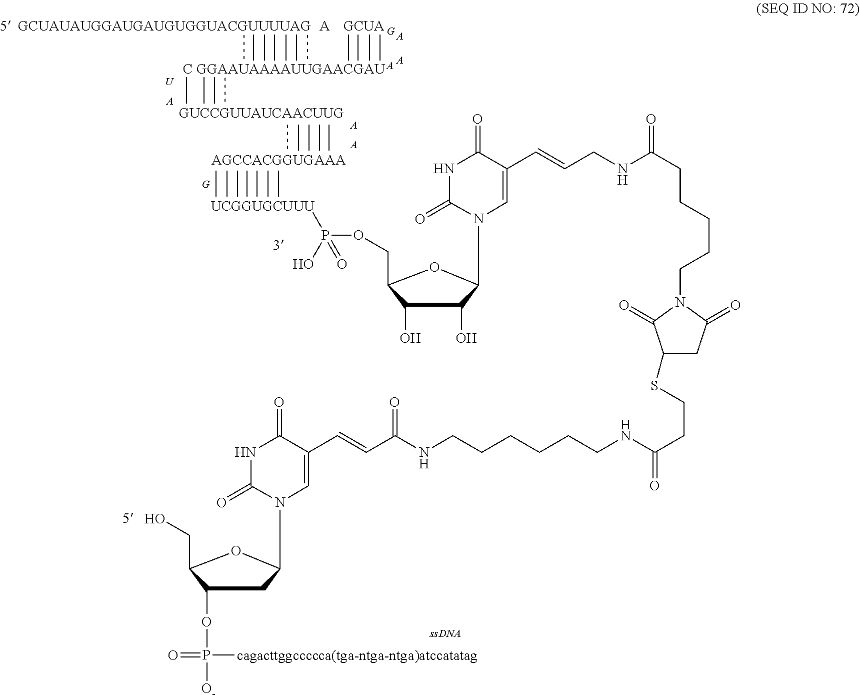

The ON-5 oligonucleotide carrying a maleimido group is incubated with 5′-SH-oligonucleotide (ON-4, 1:1 molar ratio) in 0.1 M triethylammonium acetate (TEAA) at pH 7.0 overnight at room temperature. The reaction mixture is analyzed and separated by HPLC to give the sgRNA-ssDNA.

### Example 12: ssDNA-lgRNA (SEQ ID NO: 73)

The ssDNA oligonucleotide carrying a maleimido group at its 3′-end is incubated with the 5′-SH-lgRNA (1:1 molar ratio) in 0.1 M triethylammonium acetate (TEAA) at pH 7.0 overnight at room temperature. The reaction mixture is analyzed and separated by HPLC to give ssDNA-lgRNA.

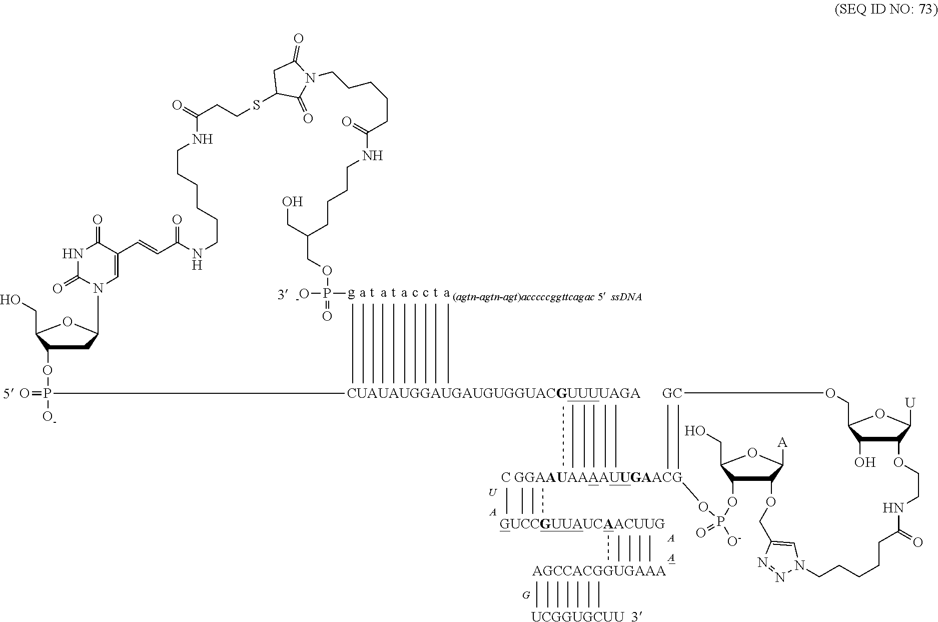

### Example 13: Cys-SpCas9::ssDNA-lgRNA Complex

The PEGylated Cys-SpCas9 protein and ssDNA-lgRNA are preincubated in a 1:2 molar ratio in the cleavage buffer to reconstitute the Cas9::ssDNA-lgRNA complex. The PEGylated RNP complex(es) is used for DNA cleavages according to reported procedures without further purification.

### Example 14: Cys-SpCas9-Nickase (H840A)::ssDNA-lgRNA Complex

A Cys-SpCas9-nickase (H840A) conjugate and ssDNA-lgRNA are preincubated in a 1:2 molar ratio in the cleavage buffer to reconstitute the Cas9::ssDNA-lgRNA complex. The RNP complex(es) is used for DNA cleavages according to reported procedures without further purification.

### Example 15: CELLULAR TRANSFECTIONS, and ASSAYS of

Cas9::ssDNA-lgRNA

a. Transfection with cationic lipids (Liu, D. et al. Nature Biotechnology 2015, 33, 73-80):

Purified synthetic ssDNA-lgRNA or a mixture of synthetic ssDNA-lgRNAs is incubated with purified Cas9 protein for 5 min, and then complexed with the cationic lipid reagent in 25 L OPTIMEM. The resulting mixture is applied to the cells for 4 h at 37° C.

b. Transfection with cell-penetrating peptides (Kim, H. et al. Genome Res. 2014, 24: 1012-1019):

Cell-penetrating peptide (CPP) is conjugated to a purified recombinant Cys-Cas9 protein by drop wise mixing of 1 mg Cas9 protein (2 mg/mL) with 50 g 4-maleimidobutyryl-GGGRRRRRRRRRLLLL (m9R; 2 mg/mL) in PBS (pH 7.4) followed by incubation on a rotator at room temperature for 2 h. To remove unconjugated m9R, the samples are dialyzed against DPBS (pH 7.4) at 4° C. for 24 h using 50 kDa molecular weight cutoff membranes. Cys-Cas9-m9R protein is collected from the dialysis membrane and the protein concentration is determined using the Bradford assay (Biorad).

Synthetic ssDNA-lgRNA or a mixture of synthetic ssDNA-lgRNAs is complexed with CPP: ssDNA-lgRNA (1 g) in 1 μl of deionized water is gently added to the C3G9R4LC peptide (9R) in lgRNA:peptide weight ratios that range from 1:2.5 to 1:40 in 100 μl of DPBS (pH 7.4). This mixture is incubated at room temperature for 30 min and diluted 10-fold using RNase-free deionized water.

150 μl Cys-Cas9-m9R (2 M) protein is mixed with 100 μl ssDNA-lgRNA:9R (10:50 g) complex and the resulting mixture is applied to the cells for 4 h at 37° C. Cells can also be treated with Cys-Cas9-m9R and ssDNA-lgRNA:9R sequentially.

### Example 16: Anti-HBV in Cells

The antiviral assay is performed according to reported procedures (Hu, W. et al. Proc Natl Acad Sci USA 2014, 110: 11461-11466; Lin, Su. et al. Molecular Therapy-Nucleic Acids, 2014, 3, e186). Delivery to cell lines is either cationic lipid or CPP based delivery of Cys-Cas9-ssDNA-lgRNA complexes instead of plasmid transfection/transduction using gRNA/Cas9 expression vectors.

Alternatively, cells are treated with ssDNA-lgRNA and mRNA encoding Cas9 protein (ssDNA-lgRNA/mRNA-10:1) either as a mixture or sequentially in LNPs formulated with an amine-to-RNA-phosphate ratio of about 3-6 (N:P), or cells are treated with ssDNA-lgRNA in LNPs formulated with an amine-to-RNA-phosphate ratio of about 3-6 (N:P) and AAV vector encoding Cas9 protein.

### Example 17: Anti-HBV in Chimeric Mice

27The antiviral assay in HBV infected chimeric mice is performed according to a reported procedure except conjugates of Cys-Cas9-ssDNA-lgRNA RNP complexes are administrated instead of small interfering RNAs (Thi, E. P. and et al. ACS Infec. Dis. 2019, 5, 725-737). All animals are bred under specific pathogen-free conditions in accordance with the ethical guidelines set forth by the National Institutes of Health for care of laboratory animals. The cDNA-uPA/SCID (cDNA-uPA (+/wt)/SCID (+/+)) hemizygote mice are generated as described. Cryopreserved human hepatocytes (2-year-old female, Hispanic, BD195, BD Biosciences) are transplanted into 2-4-week-old hemizygous cDNA-uPA/SCID mice via the spleen under anesthesia. The human hepatocytes are allowed to expand for 10-12 weeks and the replacement index are tested by measuring human albumin (h-Alb) in blood collected from tail vein using clinical chemistry analyzer (BioMajesty Series JCA-BM6050, JEOL Ltd.) with latex agglutination immunonephelometry (LZ Test “Eiken” U-ALB, Eiken Chemical Co., Ltd.). Male chimeric mice with more than 7.0 mg/mL h-Alb concentration in blood are judged as PXBmice whose replacement index is more than 70%.

PXB mice (>70% replacement index, 13-15 weeks old) are infected with HBV by intravenous injection through the tail vein with 1×10⁵ copies of HBV containing serum from previously infected animals. Eight weeks post infection, animals with HBV DNA titers greater than 1.0×10⁶ copies/mL and h-Alb greater than 7.0 mg/mL are selected (n=5 per group). Cys-Cas9-ssDNA-lgRNA complexes are dosed via the lateral tail vein in a volume of 0.2 mL per animal. Animals are euthanized at various time points by exsanguination under isoflurane anesthesia. Liver tissue is collected from the median or left lateral lobe from each animal for DNA extraction and for NGS. Editing efficiency and off-targets are determined as described (Finn, J. D. et al. Cell Reports 2018, 22, 2227-2235; Tsai, S. Q. et al. Nat. Methods 2017, 14, 607-614).

Blood is collected into serum separator tubes. Serum HBV DNA is assayed by qPCR and serum HBsAg measured by chemiluminescence enzyme immunoassay (ARCHITECT, Abbott). Serum HBeAg is also assessed using a chemiluminescence enzyme immunoassay (ARCHITECT, Abbott). Liver total and 3.5 kb HBV (pg)RNA at day 42 (study termination) are analyzed by Quantigene 2.0 b DNA assay (Affymetrix), and data is normalized to human glyceraldehyde-3-phosphate dehydrogenase (GAPDH) mRNA. Immunohistochemical analysis for HBcAg is conducted on liver sections at day 42.

Alternatively, ssDNA-lgRNA and mRNA encoding Cas9 protein (ssDNA-lgRNA/mRNA˜10:1) are administrated either as a mixture or sequentially in LNPs formulated with an amine-to-RNA-phosphate ratio of about 3-6 (N:P), or AAV vector encoding Cas9 protein and ssDNA-lgRNA in LNPs formulated with an amine-to-RNA-phosphate ratio of about 3-6 (N:P) are administrated sequentially.

### Example 18: Anti-HIV in Humanized Mice

The antiviral assay in HIV infected humanized mice is performed according to a reported procedure except Cys-Cas9-ssDNA-lgRNA RNP complexes are administrated instead of the AAV9-CRISPR-Cas9 vector (Dash, P. K. and et al. Nat. Comm. 2019, 10, 1-20).

CD34+HSC are enriched from human cord blood or fetal liver cells using immune-magnetic beads. CD34+cell purity was >90% by flow cytometry. Cells are transplanted into newborn mice irradiated at 1 Gy using a RS-2000×-Ray Irradiator by intrahepatic (i.h.) injection of 50,000 cells/mouse in 20 μl phosphate-buffered saline (PBS) with a 30-gauge needle. At 18 weeks of age, NSG-hu mice are infected intraperitoneally (i.p.) with HIV-1_{NL4-3} at 10⁴ tissue culture infective dose₅₀ (TCID₅₀)/ml.

Cys-Cas9-ssDNA-lgRNA complexes are dosed via the lateral tail vein in a volume of 0.2 mL per animal. HIV-1 nucleic acids are detected by ddPCR and editing efficiency and off-targets are determined as described.

Alternatively, ssDNA-lgRNA and mRNA encoding Cas9 protein (ssDNA-lgRNA/mRNA˜10:1) are administrated either as a mixture or sequentially in LNPs formulated with an amine-to-RNA-phosphate ratio of about 3-6 (N:P), or AAV vector encoding Cas9 protein and ssDNA-lgRNA in LNPs formulated with an amine-to-RNA-phosphate ratio of about 3-6 (N:P) are administrated sequentially.

### Example 19: AAV-EFS_CMV::NLS-SaCas9-NLS-polyA;U6-tracrRNA Plasmid

5′-caccact tatac ctaaa attac agaat ctact aaaac aaggc aaaat gccgt gttta tctcg tcaac ttgtt ggcga gattt tt-3′ (top, SEQ ID NO: 74)

5′-ggccaa aaatc tcgcc aacaa gttga cgaga taaac acggc atttt gcctt gtttt agtag attct gtaat tttag gtata agt-3′ (bottom, SEQ ID NO: 75)

The top and bottom strands of oligonucleotides for cDNA encoding tracrRNA of SaCas9 system are synthesized using DNA phosphoramidite monomers with routine protection of the A, G and C nucleobases and unprotected thymine. 0.45 M tetrazole in acetonitrile is used as the coupling agent, acetic anhydride as the capping agent and 0.1 M iodine as the oxidizing agent. Oligonucleotide synthesis is carried out on an Applied Biosystems 394 automated DNA/RNA synthesizer using the standard 1.0 μmole DNA phosphoramidite cycle. Nucleoside on CPG support is packed into a twist column. All β-cyanoethyl phosphoramidite monomers are dissolved in anhydrous acetonitrile to a concentration of 0.1 M immediately prior to use. Stepwise coupling efficiencies are determined by automated trityl cation conductivity monitoring and in all cases are >99%.

Viral plasmids are produced utilizing standard recombinant DNA cloning techniques.

The above cDNA strands (1:1) are phosphorylated and annealed, and cloned into a linearized pX601-AAV-EFS_CMV::NLS-SaCas9-NLS-polyA3×HA-bGHpA;U6::BsaI-sgRNA expression plasmid vector (Addgene gene plasmid #61591) as following. The AAV vector is digested with BsaI and NotI to remove the insert of sgRNA scaffold, treated with antarctic phosphatase, and purified with a Quick nucleotide removal kit (QIAGEN). An equal amount of complementary oligonucleotide is mixed in T4 polynucleotide kinase (PNK) buffer for annealing. These annealed seed pairs are phosphorylated with T4 PNK and ligated into the BsaI- and NotI digested AAV using T7 DNA ligase.

### Example 20: Virus Production

The AAV::SaCas9 virus is packaged using a triple plasmid transfection method as described (Chew, et al. Nature Methods, 2016). 293FT cells (Life Technologies) are plated in growth media consisting of DMEM+glutaMAX+pyruvate+10% FBS (Life Technologies), supplemented with 1×MEM non-essential amino acids (Gibco) in 150 mm plates. Confluency at transfection is between 70-90%, 20 μg of pHelper plasmid, 10 μg of pRepCap plasmid (encoding capsid proteins), and 10 μg of AAV plasmid carrying the construct of interest are mixed in 500 μL of DMEM, and 200 μg of PEI “MAX” (Polysciences) (40 kDa, 1 mg/mL in H2O, pH 7.1) are added for PEI:DNA mass ratio of 5:1. The mixture is incubated for 15 minutes, and transferred drop-wise to the cell media. The day after transfection, media is changed to DMEM+glutamax+pyruvate+2% FBS. Cells are harvested 48-72 hours after transfection by scrapping or dissociation with 1×PBS (pH7.2)+5 mM EDTA, and pelleted at 1500 g for 12 min. Cell pellets are resuspended in 1-5 mL of lysis buffer (Tris HCl pH 7.5+2 mM MgCl2+150 mM NaCl), and freeze-thawed 3× between dry-ice-ethanol bath and 37° C. water bath. Cell debris is clarified via 4000 g for 5 minutes, and the supernatant is collected.

The collected AAV supernatant is treated with 50 U/mL Benzonase and 1 U/mL Riboshredder for 30 minute at 37° C. After incubation, the lysate is concentrated to <3 mL by ultrafiltration with Amicon Ultra-15 (50 kDa MWCO) (Millipore), and loaded on top of a discontinuous density gradient consisting of 2 mL each of 15%, 25%, 40%, 60% Optiprep (Sigma-Aldrich) in an 11.2 mL Optiseal polypropylene tube (Beckman-Coulter). The tubes are ultracentrifuged at 58000 rpm, at 18° C., for 1.5 hours, on an NVT65 rotor. The 40% fraction is extracted, and dialyzed with 1×PBS (pH 7.2) supplemented with 35 mM NaCl, using Amicon Ultra-15 (50 kDa or 100 kDa MWCO) (Millipore). The purified AAVs are stored at −80° C. as 25 μL aliquots.

AAV titers (vector genomes) are quantified via hydrolysis probe qPCR against standard curves generated from linearized parental AAV plasmids.

### Example 21: Generation of Cas9-tracrRNA Stable Cells and Transfection with crRNA(s)

Cells are plated at 2×10⁴ per well in a 96-well plate in 100 μL of growth media. Purified AAVs are applied at confluency of 70-90%. Culture media is replaced with fresh growth media the next day, and the cells are incubated at 37° C. and 5% CO₂ for 24 hours. Culture media is replaced with fresh media, and the cells are transfected with crRNAs either gymnotically or with transfection reagents.

### Example 22: Mice Injection

AAV vector and crRNAs are delivered to 5-6 week old male mice intravenously via lateral tail vein injection. All dosages of AAV are adjusted to 100 μL or 200 μL with sterile phosphate buffered saline (PBS), pH 7.4 (Gibco) before the injection.

### Example 23: Generation of Inducible Cas9 Stable Cells and Transfection with lgRNA(s)

Cells are transduced with Edit-R Inducible Lentiviral hEF1α-Blast-Cas9 Nuclease Particles as detailed in the manufacturer's protocol. Cells are plated at 5×10⁴ cells per well in a 24-well plate using Tet-free growth medium, incubated at 37° C. in a humidified CO₂-incubator overnight, and transduced with Edit-R Inducible Lentiviral hEF1α-Blast-Cas9 Nuclease. The incubation is continued for 4-6 hours, and the medium is replaced with Tet-free growth medium and the incubation is continued for 24-48 hours. Inducible Cas9 stable cells are selected in the medium with Tet-free selection medium containing the appropriate amount of blasticidin and expanded.

The selected inducible Cas9 stable cells are then induced in freshly prepared doxycycline solution for at least 24 hours. The cells are transfected using Lipofectamine RNAiMax and the lgRNA(s). The medium is replaced with fresh medium after 12 hours. Cells are then grown for 72 hours with the media replaced when necessary.

### EQUIVALENTS

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 or 1 to 3 items refers to groups having 1, 2, or 3 items. Similarly, a group having 1-5 or 1 to 5 items refers to groups having 1, 2, 3, 4, or 5 items, and so forth.

All patents, patent applications, provisional applications, and publications referred to or cited herein are incorporated by reference in their entirety, including all figures and tables, to the extent they are not inconsistent with the explicit teachings of this specification.

DNA sequence of Cys-SpCas9 protein:
(SEQ ID NO: 76)
tgcgacaagaagtacagcatcggcctggacatcggcaccaactctgtgggctgggccgtgatcaccgacgagtacaaggtgc
ccagcaagaaattcaaggtgctgggcaacaccgaccggcacagcatcaagaagaacctgatcggagccctgctgttcgaca
gcggcgaaacagccgaggccacccggctgaagagaaccgccagaagaagatacaccagacggaagaaccggatcgcct
atctgcaagagatcttcagcaacgagatggccaaggtggacgacagcttcttccacagactggaagagtccttcctggtggaa
gaggataagaagcacgagcggcaccccatcttcggcaacatcgtggacgaggtggcctaccacgagaagtaccccaccatc
taccacctgagaaagaaactggtggactgcaccgacaaggccgacctgcggctgatctatctggccctggcccacatgatca
agttccggggccacttcctgatcgagggcgacctgaaccccgacaacagcgacgtggacaagctgttcatccagctggtgca
gacctacaaccagctgttcgaggaaaaccccatcaacgcctgccggcgtggacgccaaggccatcctgtctgccagactgagc
aagagcagacggctggaaaatctgatcgcccagctgcccggcgagaagaagaatggcctgttcggaaacctgattgccctg
agcctgggcctgacccccaacttcaagagcaacttcgacctggccgaggatgccaaactgcagctgagcaaggacacctac
gacgacgacctggacaacctgctggcccagatcggcgaccagtacgccgacctgtttctggccgccaagaacctgtccgac
gccatcctgctgagcgacatcctgagagtgaacaccgagatcaccaaggcccccctgagcgcctctatgatcaagagatacg
acgagcaccaccaggacctgaccctgctgaaagctctcgtgcggcagcagctgcctgagaagtacaaagagattttcttcgac
cagagcaagaacggctacgccggctacattgacggcggagccagccaggaagagttctacaagttcatcaagcccatcctg
gaaaagatggacggcaccgaggaactgctcgtgaagctgaacagagaggacctgctgcggaagcagcggaccttcgacaa
cggcagcatcccccaccagatccacctgggagagctgcacgccattctgcggcggcaggaagatttttacccattcctgaag
gacaaccgggaaaagatcgagaagatcctgaccttccgcatcccctactacgtgggccctctggccaggggaaacagcaga
ttcgcctggatgaccagaaagtgcgaggaaaccatcaccccctggaacttcgaggaagtggtggacaagggcgcttccgcc
cagagcttcatcgagcggatgaccaacttcgataagaacctgcccaacgagaaggtgctgcccaagcacagcctgctgtacg
agtacttcaccgtgtataacgagctgaccaaagtgaaatacgtgaccgagggaatgagaaagcccgccttcctgagcggcga
gcagaaaaaggccatcgtggacctgctgttcaagaccaaccggaaagtgaccgtgaagcagctgaaagaggactacttcaa
gaaaatcgagtgcttcgactccgtggaaatctccggcgtggaagatcggttcaacgcctccctgggcacataccacgatctgc
tgaaaattatcaaggacaaggacttcctggacaatgaggaaaacgaggacattctggaagatatcgtgctgaccctgacactgt
ttgaggacagagagatgatcgaggaacggctgaaaacctatgcccacctgttcgacgacaaagtgatgaagcagctgaagc
ggcggagatacaccggctggggcaggctgagccggaagctgatcaacggcatccgggacaagcagtccggcaagacaat
cctggatttcctgaagtccgacggcttcgccaacagaaacttcatgcagctgatccacgacgacagcctgacctttaaagagg
acatccagaaagcccaggtgtccggccagggcgatagcctgcacgagcacattgccaatctggccggcagccccgccatta
agaagggcatcctgcagacagtgaaggtggtggacgagctcgtgaaagtgatgggccggcacaagcccgagaacatcgtg
atcgaaatggccagagagaaccagaccacccagaagggacagaagaacagccgcgagagaatgaagcggatcgaagag
ggcatcaaagagctgggcagccagatcctgaaagaacaccccgtggaaaacacccagctgcagaacgagaagctgtacct
gtactacctgcagaatggggggatatgtacgtggaccaggaactggacatcaaccggctgtccgactacgatgtggaccat
atcgtgcctcagagctttctgaaggacgactccatcgacaacaaggtgctgaccagaagcgacaagaaccggggcaagagc
gacaacgtgccctccgaagaggtcgtgaagaagatgaagaactactggcggcagctgctgaacgccaagctgattacccag
agaaagttcgacaatctgaccaaggccgagagaggcggcctgagcgaactggataaggccggcttcatcaagagacagctg
gtggaaacccggcagatcacaaagcacgtggcacagatcctggactcccggatgaacactaagtacgacgagaatgacaag
ctgatccgggaagtgaaagtgatcaccctgaagtccaagctggtgtccgatttccggaaggatttccagttttacaaagtgcgc
gagatcaacaactaccaccacgcccacgacgcctacctgaacgccgtcgtgggaaccgccctgatcaaaaagtaccctaag
ctggaaagcgagttcgtgtacggcgactacaaggtgtacgacgtgcggaagatgatcgccaagagcgagcaggaaatcggc
aaggctaccgccaagtacttcttctacagcaacatcatgaactttttcaagaccgagattaccctggccaacggcgagatccgg
aagcggcctctgatcgagacaaacggcgaaaccggggagatcgtgtgggataagggccgggattttgccaccgtgcggaa
agtgctgagcatgccccaagtgaatatcgtgaaaaagaccgaggtgcagacaggcggcttcagcaaagagtctatcctgccc
aagaggaacagcgataagctgatcgccagaaagaaggactgggaccctaagaagtacggcggcttcgacagccccaccgt
ggcctattctgtgctggtggtggccaaagtggaaaagggcaagtccaagaaactgaagtgcgtgaaagagctgctggggatc
accatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcag
aagggaaacgaactggccctgccctccaaatatgtgaacttcctgtacctggccagccactatgagaagctgaagggctcccc
cgaggataatgagcagaaacagctgtttgtggaacagcacaagcactacctggacgagatcatcgagcagatcagcgagttc
tccaagagagtgatcctggccgacgctaatctggacaaagtgtgtccgcctacaacaagcaccgggataagcccatcagag
agcaggccgagaatatcatccacctgtttaccctgaccaatctgggagcccctgccgccttcaagtactttgacaccaccatcg
accggaagaggtacaccagcaccaaagaggtgctggacgccaccctgatccaccagagcatcaccggcctgtacgagaca
cggatcgacctgtctcagctgggaggcgac
Sequence of Cys-SpCas9-NLS protein:
(SEQ ID NO: 77)
CDKKYSIGLDIGTNSVGWAVITDEYKVPSKKFKVLGNTDRHSIKKNLIGALLFDSGETAEATRL
KRTARRRYTRRKNRIAYLQEIFSNEMAKVDDSFFHRLEESFLVEEDKKHERHPIFGNIVDEVAY
HEKYPTIYHLRKKLVCSTDKADLRLIYLALAHMIKFRGHFLIEGDLNPDNSDVDKLFIQLVQTY
NQLFEENPINACGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTFRIP
YYVGPLARGNSRFAWMTRKCEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDSPTV
AYSVLVVAKVEKGKSKKLKCVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHY
LDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDAYPYDVPDYASLGSGSPKKKRKVD

What is claimed is:

1. A conjugate of CRISPR-Cas protein-guide RNA(s) complex, comprising
a) an lgRNA-Cas protein (RNP) complex and
b) one or more molecules selected from the group consisting of PEG, non-PEG polymers, ligands of cellular receptors, lipids, oligonucleotides, antibodies, polysaccharides, glycans and peptides,
wherein said one or more molecules are chemically linked to said RNP complex.

2-38. (canceled)

39. Said conjugated oligonucleotide of claim 1 comprising a sequence to introduce one or more stop codons selected from the sequence group consisting of 5′-(tga)-3′, 5′-(taa)-3′, 5′-(tag)-3′, 5′-(tga-ntga-ntga)-3′, 5′-(tga-ntga-ntaa)-3′, 5′-(tga-ntga-ntag)-3′, 5′-(tga-ntaa-ntga)-3′, 5′-(tga-ntaa-ntaa)-3′, 5′-(tga-ntaa-ntag)-3′, 5′-(tga-ntga-ntga)-3′, 5′-(tga-ntga-ntaa)-3′, 5′-(tga-ntga-ntag)-3′, 5′-(taa-ntga-ntga)-3′, 5′-(taa-ntga-ntaa)-3′, 5′-(taa-ntga-ntag)-3′, 5′-(taa-ntaa-ntga)-3′, 5′-(taa-ntaa-ntaa)-3′, 5′-(taa-ntaa-ntag)-3′, 5′-(taa-ntga-ntga)-3′, 5′-(taa-ntga-ntaa)-3′, 5′-(taa-ntga-ntag)-3′, 5′-(tag-ntga-ntga)-3′, 5′-(tag-ntga-ntaa)-3′, 5′-(tag-ntga-ntag)-3′, 5′-(tag-ntaa-ntga)-3′, 5′-(tag-ntaa-ntaa)-3′, 5′-(tag-ntaa-ntag)-3′, 5′-(tag-ntga-ntga)-3′, 5′-(tag-ntga-ntaa)-3′, 5′-(tag-ntga-ntag)-3′, wherein n is any nucleotide, and said more stop codons comprises repetitive said sequence separated by absent or more nucleotides in between or different said sequences separated by absent or more nucleotides in between.

40. Said conjugated oligonucleotide of claim 1 comprising one or more aptamers.

41. Said conjugate of CRISPR-Cas protein-lgRNA(s) complex of claim 1, wherein the chemical linkages are covalent, and each optionally biodegradable in targeted cells and tissues.

42. Said conjugate of CRISPR-Cas protein-lgRNA(s) complex of claim 41, wherein the chemical linkages are redox-responsive disulfide bond.

43. Said conjugate of CRISPR-Cas protein-lgRNA(s) complex of claim 41, wherein the biodegradable covalent linkage is selected from the group consisting of acetal [OC(RR′)O], carboxylic acid ester [R(C═O)—O], thioester [R(C═O)—S], carbamate ester [N(RR′)(C═O)—O], and carbonate ester [RO(C═O)—O], wherein each of R and R′ is an optionally substituted alkyl, or an optionally substituted aryl, or an optionally substituted heteroaryl.

44. A method of gene editing comprising the following steps:
a) delivering to targeted cells conjugates of CRISPR-Cas protein-lgRNA complex of claim 1;
b) cleaving DNA to be edited, leading to a double strand break or a nick;
c) hybridizing the resulting single DNA strand of the cleavage product with the 3′-homology arm of conjugated donor template and extending the 3′-end of complementary broken strand using said template to edit the target gene by introducing insertions, deletions or point mutations included in the gene editing sequence of conjugated oligonucleotide;
d) recycling the RNP complexes.

45. Said method of claim 44 for treatment of chronic viral infections comprising the following steps:
a) delivering to infected cells conjugates of CRISPR-Cas protein-lgRNA complex of claim 1;
b) cleaving viral episomal, viral integrated DNA or both, leading to a double strand break or a nick;
c) hybridizing one of the resulting single DNA strand of the cleavage product with the 3′-homology arm of conjugated donor template and extending the 3′-end of complementary broken strand using said template to deactivate the viral gene by introducing insertions, deletions or point mutations included in the gene editing sequence;
d) recycling the RNP complexes.

46. Said chronic viral infection in claim 45 is HBV, HIV or herpesvirus infection.

47. A method of delivering conjugates of a CRISPR RNP complex(es) of claim 1 to cells or animals comprising:
a) delivering a tissue tropic viral vector encoding a Cas protein comprising recognition domains and endonuclease domains and
b) delivering guide RNA(s) selected from the group consisting of lgRNA(s), lgRNA conjugate(s), dual guide RNAs comprising a ligated tracrRNA or its conjugate(s), dual guide RNAs comprising a crRNA conjugate(s) and a ligated tracrRNA, and guide RNAs comprising a crRNA conjugate(s), in an aqueous solution gymnotically or with transfection reagents or in a non-viral carrier;
wherein a) and b) can be co-injections or separate injections.

48. Said method of claim 47 further comprises: c) delivering one or more donor nucleic acid sequence for correction, insertion or replacement of a target gene, and the sequence is optionally conjugated with cell targeting ligands, antibodies, or aptamers, either in an aqueous solution gymnotically or with transfection reagents or in a non-viral carrier.

49. Said method of claim 47, wherein said guide RNA conjugate(s) of b) comprises guide RNA(s) and one or more conjugated ssDNA templates for DNA repair and delivered either in an aqueous solution gymnotically or with transfection reagents or in a non-viral carrier.

50. Said method of claim 49, wherein said conjugated ssDNA template is replaced with a double strand DNA (dsDNA) covalently linked to guide RNA(s) via a linker to either of its double strands. The said linker is a nucleotide linker or an nNt-linker.

51. Said method of claim 47, wherein said Cas protein of a) is delivered as its mRNA either in an aqueous solution gymnotically or with transfection reagents or in a non-viral carrier.

52. Said method of claim 47, wherein:
said tissue tropic viral vector of a) encodes both a tracrRNA and a Cas protein comprising recognition domains and endonuclease domains;
and said guide RNA of b) is a crRNA conjugate.

53. Said method of claim 52 further comprises: c). delivering a donor nucleic acid sequence for correction, insertion or replacement of a target gene, and the sequence optionally conjugated with cell targeting ligands, antibodies, or aptamers, either in an aqueous solution gymnotically or with transfection reagents or in a non-viral carrier, wherein a), b) and c) can be co-injections or separate injections.

54. Said method of claim 52, wherein said crRNA conjugate(s) of b) comprises a crRNA and one or more ssDNA template for DNA repair conjugated at either 5′-end or 3′-end of crRNA and delivered either in an aqueous solution gymnotically or with transfection reagents or in a non-viral carrier.

55. Said method of claim 54, wherein said conjugated ssDNA template is replaced with a double strand DNA (dsDNA) covalently linked to crRNA(s) via a linker to either of its double strands. The said linker is a nucleotide linker or an nNt-linker.

56. Said method of claim 52, wherein the expression of said Cas protein and tracrRNA is optionally under the control of a single or a plurality of switchable transcription promotor and/or enhancer and/or depressor.


SEQUENCE LISTING (77 sequences)

SEQ ID NO: 1 - RNA, 42 nt, Streptococcus pyogenes
  misc_feature (1)..(20): n is a, c, g, or u
nnnnnnnnnnnnnnnnnnnnguuuuagagcuaugcuguuuug

SEQ ID NO: 2 - RNA, 81 nt, Streptococcus pyogenes
ggaaccauucaaaacagcauagcaaguuaaaauaaggcuaguccguuaucaacuugaaaaaguggcaccgagucggugcuu

SEQ ID NO: 3 - RNA, 94 nt, Artificial Sequence
  lgRNA, nn (32, 33) is either base-paired  or not, and joined by non-nucleotide linker between the two, n32 (A) and n33 (B).
  misc_feature (1)..(20): n is a, c, g, or u
  misc_feature (32)..(33): n is a, c, g, or u
nnnnnnnnnnnnnnnnnnnnguuuuagagcunnagcaaguuaaaauaaggcuaguccguuaucaacuugaaaaaguggcaccgagucggugcuu

SEQ ID NO: 4 - RNA, 100 nt, Artificial Sequence
  sgRNA: crRNA and tracrRNA are fused as a single whole RNA by a tetraloop, gaaa.
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 5 - RNA, 94 nt, Artificial Sequence; identical to SEQ ID NO: 3
  lgRNA formed by two ligations between nn (32, 33) of crgRNA and tracrgRNA, and between a70 and a71 in tracrgRNA.
  misc_feature (1)..(20): n is a, c, g, or u
  misc_feature (32)..(33): n is a, c, g, or u

SEQ ID NO: 6-10 - skipped (5 entries)

SEQ ID NO: 11 - DNA, 94 bp, Artificial Sequence
  gRNA inserted with an aptamer between a32 and u33. The aptamer sequence is not shown.
  misc_feature (1)..(20): n is a, c, g, t or u
nnnnnnnnnnnnnnnnnnnnguuuuagagcuauagcaaguuaaaauaaggcuaguccguuaucaacuugaaaaaguggcaccgagucggugcuu

SEQ ID NO: 12 - DNA, 92 bp, Artificial Sequence
  gRNA inserted with two aptamers, positioned between a32 and u33 and between g69 and a70. The two aptamer sequences are not shown.
  misc_feature (1)..(20): n is a, c, g, t or u
nnnnnnnnnnnnnnnnnnnnguuuuagagcuauagcaaguuaaaauaaggcuaguccguuaucaacuugaaaguggcaccgagucggugcuu

SEQ ID NO: 13 - DNA, 94 bp, Artificial Sequence
  lgRNA inserted with two chemically ligated aptamers by nNt-linkers, positioned between n32 and n33 and between a70 and a71. The two aptamer sequences are not shown.
  misc_feature (1)..(20): n is a, c, g, t or u
  misc_feature (32)..(33): n is a, c, g, t or u
nnnnnnnnnnnnnnnnnnnnguuuuagagcunnagcaaguuaaaauaaggcuaguccguuaucaacuugaaaaaguggcaccgagucggugcuu

SEQ ID NO: 14 - DNA, 62 bp, Artificial Sequence
  Chemically ligated tracrRNA inserted with two aptamers by nNt-linkers, positioned at 5′-end  and n33 and between a38 and a39. The two aptamer sequences are not shown.
  misc_feature (1)..(1): n is a, c, g, t or u
nagcaaguuaaaauaaggcuaguccguuaucaacuugaaaaaguggcaccgagucggugcuu

SEQ ID NO: 15 - DNA, 95 bp, Artificial Sequence
  lgRNA conjugated with a chemically ligated aptamer at  u33 (modified) and  joined by an nNt-linker positioned between u33 and n34. The aptamer sequence is not shown.
  misc_feature (1)..(20): n is a, c, g, t or u
  misc_feature (32)..(32): n is a, c, g, t or u
  misc_feature (34)..(34): n is a, c, g, t or u
nnnnnnnnnnnnnnnnnnnnguuuuagagcununagcaaguuaaaauaaggcuaguccguuaucaacuugaaaaaguggcaccgagucggugcuu

SEQ ID NO: 16 - DNA, 36 bp, Streptococcus pyogenes
  misc_feature (1)..(20): n is a, c, g, t or u
nnnnnnnnnnnnnnnnnnnnguuuuagagcuaugcu

SEQ ID NO: 17 - DNA, 66 bp, Streptococcus pyogenes
agcauagcaaguuaaaauaaggcuaguccguuaucaacuugaaaaaguggcaccgagucggugcuu

SEQ ID NO: 18 - DNA, 47 bp, Artificial Sequence
  lgRNA (partial sequence) formed by chemical ligation bwteen tracrRNA and crRNA. The nNt-linker is positioned bwteen c15 (3′-end of tracrRNA) and g16 (5′-end of crRNA).
  misc_feature (28)..(47): n is a, c, g, t or u
ggaagggaacccuccgguggguuaaagnnnnnnnnnnnnnnnnnnnn

SEQ ID NO: 19 - DNA, 94 bp, Artificial Sequence; identical to SEQ ID NO: 13
  lgRNA, nn (32, 33) is either base-paired  or not, and joined by non-nucleotide linker between the two, n32 (A) and n33 (B). GalAc ligands are conjugated at 3′-end of lgRNA via a Linker.
  misc_feature (1)..(20): n is a, c, g, t or u
  misc_feature (32)..(33): n is a, c, g, t or u

SEQ ID NO: 20 - DNA, 47 bp, Artificial Sequence; identical to SEQ ID NO: 18
  lgRNA (partial sequence) formed by chemical ligation bwteen tracrRNA and crRNA. The nNt-linker of lgRNA is positioned bwteen c15 (3′-end of tracrRNA) and g16 (5′-end of crRNA). GalAc ligands are conjugated at 3′-end of lgRNA via a second Linker.
  misc_feature (28)..(47): n is a, c, g, t or u

SEQ ID NO: 21 - DNA, 42 bp, Artificial Sequence
  Truncated crRNA with a general spacer.
  misc_feature (1)..(20): n is a, c, g, t or u
nnnnnnnnnnnnnnnnnnnnguuuuagagcuaugcuguuuug

SEQ ID NO: 22 - DNA, 81 bp, Artificial Sequence
  GalAc ligands are conjugated at 3′-end of tracrRNA via a Linker.
ggaaccauucaaaacagcauagcaaguuaaaauaaggcuaguccguuaucaacuugaaaaaguggcaccgagucggugcuu

SEQ ID NO: 23 - DNA, 42 bp, Artificial Sequence; identical to SEQ ID NO: 21
  A cholesterol is conjugated at 3′-end of crRNA via a Linker.
  misc_feature (1)..(20): n is a, c, g, t or u

SEQ ID NO: 24 - DNA, 42 bp, Artificial Sequence; identical to SEQ ID NO: 21
  Tocopherol is conjugated at 3′-end of crRNA via a Linker.
  misc_feature (1)..(20): n is a, c, g, t or u

SEQ ID NO: 25 - DNA, 42 bp, Artificial Sequence; identical to SEQ ID NO: 21
  An aptamer is conjugated at 3′-end of crRNA via a Linker. The aptamer sequence is not shown.
  misc_feature (1)..(20): n is a, c, g, t or u

SEQ ID NO: 26 - DNA, 42 bp, Artificial Sequence; identical to SEQ ID NO: 21
  An antibody is conjugated at 3′-end of crRNA via a Linker. The amino acid  sequence of the antibody is not shown.
  misc_feature (1)..(20): n is a, c, g, t or u

SEQ ID NO: 27 - DNA, 25 bp, Artificial Sequence
  General scheme of a target DNA to be edited. Its non-target strand is removed.
  misc_feature (1)..(20): n is a, c, g, or t
  misc_feature (24)..(25): n is a, c, g, or t
nnnnnnnnnnnnnnnnnnnntggnn

SEQ ID NO: 28 - DNA, 32 bp, Artificial Sequence
  synthetic, ssDNA template at 3′-end
  misc_feature (1)..(20): n is a, c, g, t or u
  misc_feature (32)..(32): n is a, c, g, t or u
nnnnnnnnnnnnnnnnnnnnguuuuagagcun

SEQ ID NO: 29 - DNA, 62 bp, Artificial Sequence; identical to SEQ ID NO: 14
  synthetic, ssDNA template at 5′-end
  misc_feature (1)..(1): n is a, c, g, t or u

SEQ ID NO: 30 - DNA, 94 bp, Artificial Sequence; identical to SEQ ID NO: 13
  synthetic, ssDNA at nNt-linker_1 between crRNA and tracrRNA.
  misc_feature (1)..(20): n is a, c, g, t or u
  misc_feature (32)..(33): n is a, c, g, t or u

SEQ ID NO: 31 - DNA, 94 bp, Artificial Sequence; identical to SEQ ID NO: 13
  synthetic, ssDNA at nNt-linker_2 between a70 and a71.
  misc_feature (1)..(20): n is a, c, g, t or u
  misc_feature (32)..(33): n is a, c, g, t or u

SEQ ID NO: 32 - DNA, 94 bp, Artificial Sequence; identical to SEQ ID NO: 13
  synthetic, ssDNA at 3′-end
  misc_feature (1)..(20): n is a, c, g, t or u
  misc_feature (32)..(33): n is a, c, g, t or u

SEQ ID NO: 33 - DNA, 61 bp, Artificial Sequence
  synthetic, ssDNA at 3′-end of ligated tracrRNA
agcaaguuaaaauaaggcuaguccguuaucaacuugaaaaaguggcaccgagucggugcuu

SEQ ID NO: 34 - DNA, 94 bp, Artificial Sequence; identical to SEQ ID NO: 13
  synthetic, ssDNA at 5′-end
  misc_feature (1)..(20): n is a, c, g, t or u
  misc_feature (32)..(33): n is a, c, g, t or u

SEQ ID NO: 35 - DNA, 144 bp, Artificial Sequence
  l2gRNA formed by two ligations between n32 of crRNA and n33 of tracrRNA, and between a70 and a71 in tracrRNA. An ssDNA is conjugated at the 5′-end of the l2gRNA.
  misc_feature (1)..(70): n is a, c, g, t or u
  misc_feature (82)..(83): n is a, c, g, t or u
nnnnnnnnnnnnnnnnnnnnnnnnnnnnnnnnnnnnnnnnnnnnnnnnnnnnnnnnnnnnnnnnnnnnnnguuuuagagcunnagcaaguuaaaauaaggcuaguccguuaucaacuugaaaaaguggcaccgagucggugcuu

SEQ ID NO: 36 - DNA, 145 bp, Artificial Sequence
  lgRNA formed by two ligations between nn (32, 33) of crRNA and tracrRNA, and between a70 and a71 in tracrRNA. An ssDNA is conjugated at the triazole nNt-Linker between the u33 and the 3′-end of ssDNA.
  misc_feature (1)..(20): n is a, c, g, t or u
  misc_feature (32)..(32): n is a, c, g, t or u
  misc_feature (34)..(34): n is a, c, g, t or u
  misc_feature (96)..(145): n is a, c, g, t or u
nnnnnnnnnnnnnnnnnnnnguuuuagagcununagcaaguuaaaauaaggcuaguccguuaucaacuugaaaaaguggcaccgagucggugcuunnnnnnnnnnnnnnnnnnnnnnnnnnnnnnnnnnnnnnnnnnnnnnnnnn

SEQ ID NO: 37 - DNA, 144 bp, Artificial Sequence
  l2gRNA formed by two ligations between n32 of crRNA and n33 of tracrRNA, and between a70 and a71 in tracrRNA. An ssDNA is conjugated at the 3′-end of the l2gRNA.
  misc_feature (1)..(20): n is a, c, g, t or u
  misc_feature (32)..(33): n is a, c, g, t or u
  misc_feature (95)..(144): n is a, c, g, t or u
nnnnnnnnnnnnnnnnnnnnguuuuagagcunnagcaaguuaaaauaaggcuaguccguuaucaacuugaaaaaguggcaccgagucggugcuunnnnnnnnnnnnnnnnnnnnnnnnnnnnnnnnnnnnnnnnnnnnnnnnnn

SEQ ID NO: 38 - DNA, 119 bp, Artificial Sequence
  lgRNA formed by two ligations between u33 of crRNA and n34 of tracrRNA, and between a71 and a72 in tracrRNA. An ssDNA is conjugated at the ligation site between nn (32, 33) via an nNt-Linker between the 5′-end of ssDNA and u33.
  misc_feature (1)..(20): n is a, c, g, t or u
  misc_feature (32)..(32): n is a, c, g, t or u
  misc_feature (34)..(34): n is a, c, g, t or u
  misc_feature (96)..(119): n is a, c, g, t or u
nnnnnnnnnnnnnnnnnnnnguuuuagagcununagcaaguuaaaauaaggcuaguccguuaucaacuugaaaaaguggcaccgagucggugcuunnnnnnnnnnnnnnnnnnnnnnnn

SEQ ID NO: 39 - DNA, 194 bp, Artificial Sequence
  An ssDNA (n145 to n194) is conjugated at the 3′-end of lgRNA via a triazole nNt-Linker between the 3′-end of lgRNA and the 5′-end of ssDNA, and a second ssDNA s conjugated at the 5′-end of lgRNA.
  misc_feature (1)..(70): n is a, c, g, t or u
  misc_feature (82)..(83): n is a, c, g, t or u
  misc_feature (145)..(194): n is a, c, g, t or u
nnnnnnnnnnnnnnnnnnnnnnnnnnnnnnnnnnnnnnnnnnnnnnnnnnnnnnnnnnnnnnnnnnnnnnguuuuagagcunnagcaaguuaaaauaaggcuaguccguuaucaacuugaaaaaguggcaccgagucggugcuunnnnnnnnnnnnnnnnnnnnnnnnnnnnnnnnnnnnnnnnnnnnnnnnnn

SEQ ID NO: 40 - DNA, 144 bp, Artificial Sequence; identical to SEQ ID NO: 37
  An ssDNA is conjugated at the 3′-end of lgRNA via a triazole nNt-Linker between the 3′-end of lgRNA and a dsDNA. Its complementary strand is not shown.
  misc_feature (1)..(20): n is a, c, g, t or u
  misc_feature (32)..(33): n is a, c, g, t or u
  misc_feature (95)..(144): n is a, c, g, t or u

SEQ ID NO: 41 - DNA, 23 bp, HBV
ctctgctagatcccagagtgagg

SEQ ID NO: 42 - DNA, 23 bp, HBV
gctatcgctggatgtgtctgcgg

SEQ ID NO: 43 - DNA, 25 bp, HBV
tggacttctctcaattttctagggg

SEQ ID NO: 44 - DNA, 23 bp, HBV
gggggatcacccgtgtgtcttgg

SEQ ID NO: 45 - DNA, 25 bp, HBV
tatgtggatgatgtggtactggggg

SEQ ID NO: 46 - DNA, 21 bp, HBV
cctcaccatacagcactcggg

SEQ ID NO: 47 - DNA, 26 bp, HBV
gtgttggggtgagttgatgaatctgg

SEQ ID NO: 48 - DNA, 25 bp, HBV
tatgtggatgatgtggtactggggg

SEQ ID NO: 49 - DNA, 25 bp, HBV
tatatggatgatgtggtattggggg

SEQ ID NO: 50 - DNA, 25 bp, HBV
tatgtggatgatgtggtattggggg

SEQ ID NO: 51 - DNA, 25 bp, HBV
tatatagatgatgtggtactggggg

SEQ ID NO: 52 - DNA, 23 bp, HIV
gattggcagaactacacaccagg

SEQ ID NO: 53 - DNA, 23 bp, HIV
atcagatatccactgacctttgg

SEQ ID NO: 54 - DNA, 23 bp, HIV
gcgtggcctgggcgggactgggg

SEQ ID NO: 55 - DNA, 23 bp, HIV
cagcagttcttgaagtactccgg

SEQ ID NO: 56 - DNA, 23 bp, EBV
gccctggaccaacccggcccggg

SEQ ID NO: 57 - DNA, 23 bp, EBV
ggccgctgccccgctccgggtgg

SEQ ID NO: 58 - DNA, 22 bp, EBV
ggaagacaatgtgccgccatgg

SEQ ID NO: 59 - DNA, 23 bp, EBV
tctggaccagaaggctccggcgg

SEQ ID NO: 60 - DNA, 23 bp, EBV
gctgccgcggagggtgatgacgg

SEQ ID NO: 61 - DNA, 23 bp, EBV
ggtggcccaccgggtccgctggg

SEQ ID NO: 62 - DNA, 23 bp, EBV
gtcctcgagggggccgtcgcggg

SEQ ID NO: 63 - DNA, 21 bp, human
aaagaaaatatmmmtggtgtt

SEQ ID NO: 64 - DNA, 21 bp, human
aaagaaaatatctttggtgtt

SEQ ID NO: 65 - RNA, 50 nt, Artificial Sequence
  GalAcs are conjugated at 3′-end of crRNA via an triazole nNt-Linker.
gcguggccugggcgggacugguuuuaguacucuguaauuuuagguaugag

SEQ ID NO: 66 - RNA, 31 nt, Artificial Sequence
  crRNA 3′-azide
cuauauggaugaugugguacguuuuagagcu

SEQ ID NO: 67 - DNA, 61 bp, Artificial Sequence; identical to SEQ ID NO: 33
  5′-alkynyl tracrRNA 3′-maleimide.

SEQ ID NO: 68 - DNA, 92 bp, Artificial Sequence
  lgRNA 3′-maleimide. lgRNA contains a triazole nNt-Linker between u31 and a32.
cuauauggaugaugugguacguuuuagagcuagcaaguuaaaauaaggcuaguccguuaucaacuugaaaaaguggcaccgagucggugcuu

SEQ ID NO: 69 - DNA, 37 bp, Artificial Sequence
  ssDNA, 5′-thiol
  misc_feature (20)..(20): n is a, c, g, t or u
  misc_feature (24)..(24): n is a, c, g, t or u
ucagacttggcccccatgantgantgaatccatatag

SEQ ID NO: 70 - DNA, 129 bp, Artificial Sequence
  lgRNA-ssDNA conjugate with a thio-ether nNt-Linker (u93 and u94).
  misc_feature (112)..(112): n is a, c, g, t or u
  misc_feature (116)..(116): n is a, c, g, t or u
cuauauggaugaugugguacguuuuagagcuagcaaguuaaaauaaggcuaguccguuaucaacuugaaaaaguggcaccgagucggugcuuucagacttggcccccatgantgantgaatccatatag

SEQ ID NO: 71 - DNA, 101 bp, Artificial Sequence
  sgRNA 3′-maleimide.
gcuauauggaugaugugguacguuuuagagcuagaaauagcaaguuaaaauaaggcuaguccguuaucaacuugaaaaaguggcaccgagucggugcuuuu

SEQ ID NO: 72 - DNA, 138 bp, Artificial Sequence
  lgRNA-ssDNA conjugate with a thio-ether nNt-Linker (u101 and u102).
  misc_feature (121)..(121): n is a, c, g, t or u
  misc_feature (125)..(125): n is a, c, g, t or u
gcuauauggaugaugugguacguuuuagagcuagaaauagcaaguuaaaauaaggcuaguccguuaucaacuugaaaaaguggcaccgagucggugcuuuuucagacttggcccccatgantgantgaatccatatag

SEQ ID NO: 73 - DNA, 129 bp, Artificial Sequence
  ssDNA-lgRNA conjugate with a thio-ether nNt-Linker (g36 and u37). A second nNt-Linker is between u68 and a69.
  misc_feature (19)..(19): n is a, c, g, t or u
  misc_feature (23)..(23): n is a, c, g, t or u
cagacttggcccccatgantgantgaatccatatagucuauauggaugaugugguacguuuuagagcuagcaaguuaaaauaaggcuaguccguuaucaacuugaaaaaguggcaccgagucggugcuu

SEQ ID NO: 74 - DNA, 84 bp, Artificial Sequence
  cDNA encoding SaCas9 tracrRNA.
caccacttatacctaaaattacagaatctactaaaacaaggcaaaatgccgtgtttatctcgtcaacttgttggcgagattttt

SEQ ID NO: 75 - DNA, 84 bp, Artificial Sequence
  cDNA encoding SaCas9 tracrRNA.
ggccaaaaatctcgccaacaagttgacgagataaacacggcattttgccttgttttagtagattctgtaattttaggtataagt

SEQ ID NO: 76 - DNA, 4104 bp, Artificial Sequence
  DNA sequence of engineered SpCas9 protein by site-directed mutagenesis.
tgcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatcgcctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggactgcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgcctgcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagtgcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagtgcgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 77 - protein, 1392 aa, Artificial Sequence
  Sequence of an engineered SpCas9-NLS protein.
CDKKYSIGLDIGTNSVGWAVITDEYKVPSKKFKVLGNTDRHSIKKNLIGALLFDSGETAEATRLKRTARRRYTRRKNRIAYLQEIFSNEMAKVDDSFFHRLEESFLVEEDKKHERHPIFGNIVDEVAYHEKYPTIYHLRKKLVCSTDKADLRLIYLALAHMIKFRGHFLIEGDLNPDNSDVDKLFIQLVQTYNQLFEENPINACGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKCEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKCVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVD